(19) United States
(12) Patent Application Publication
(10) Pub. No.: US 2024/0131130 A1
(43) Pub. Date: Apr. 25, 2024

(54) COMPOSITIONS AND METHODS FOR ACTIVATING NK CELLS

(71) Applicant: The Regents of the University of California, Oakland, CA (US)

(72) Inventor: Anahid Jewett, Los Angeles, CA (US)

(21) Appl. No.: 18/277,897

(22) PCT Filed: Feb. 17, 2022
(86) PCT No.: PCT/US22/16812

§ 371 Date: Aug. 17, 2023

Related U.S. Application Data
(60) Provisional application No. 63/150,915, filed on Feb. 18, 2021.

(51) Int. Cl. A61K 39/00; C12N 5/09; C12N 15/86

(52) U.S. Cl. CPC A61K 39/0011; C12N 5/0693; C12N 15/86; A61K 2039/5152; A61K 2039/572; C12N 2529/00; C12N 2710/16143

(57) ABSTRACT

The present invention is based, in part, on cancer vaccine compositions or pharmaceutical compositions comprising cancer cells, monocytes, and/or osteoclasts that activate NK cells, and methods for using same to prevent and/or treat diseases such as cancer.

### CROSS-REFERENCE TO RELATED APPLICATION

This application claims the benefit of the following U.S. Provisional Application No. 63/150,915, filed Feb. 18, 2021, the entire contents of which are incorporated herein by reference.

### BACKGROUND OF THE INVENTION

Natural killer (NK) cells lyse and differentiate cancer stem cells/undifferentiated tumors with lower expression of MHC class I, CD54 and B7H1 and higher expression of CD44. Medium and high cytotoxic activity of peripheral-blood lymphocytes are associated with reduced cancer risk, and high NK-cell infiltration of the tumor is associated with a better prognosis, whereas low activity is associated with increased cancer risk.

Suppression of NK cells is mediated by downregulation of NK receptors in the tumor microenvironment. Function of NK cells was shown previously to be significantly reduced in tumor patients. Several in vitro NK expansion techniques have been developed to allow for a higher therapeutic cell dose. The stimulation of peripheral blood mononuclear cells (PBMCs) or purified population of NK cells with feeder cells such as K562 cells expressing interleukin (IL)-15 and 41BB ligand, EBV-TM-LCL, Wilms tumor or irradiated PBMCs have resulted in greater numbers of NK cells with adequate function. The generated NK cells expressed higher levels of NKG2D, natural cytotoxicity receptors, DNAM-1, and ICAM-1. Thus, various methods to obtain ex vivo-expanded, activated, and CD3+ T cell-depleted NK cells have been established for clinical use. In addition, it has been established that the safety and efficacy of adoptive cellular transfer of HLA-haploidentical NK cells in patients with advanced cancer. Additionally, clinical trials have shown that allogeneic NK cells play a therapeutic role in solid tumors, and are safe for transfer into patients.

Immunotherapy with NK cells has been limited due to inability to obtain sufficient numbers of highly functional NK cells, or an effective means to activate NK cells in vivo. Thus, there is a great need in the art to identify therapeutic compositions and methods for improved NK immunotherapy.

### SUMMARY OF THE INVENTION

The present invention is based, at least in part, on the discovery that a cancer vaccine comprising cancer cells deficient in one or more biomarkers are effective in activating NK cells and inducing immune response. Such deficiency results in de-differentiation of the cancer cells, and the proteins presented on their cell surface provide effective signals in activating various types of immune cells, including NK cells. Similarly, it has been determined herein that a monocyte and/or an osteoclast deficient in one or more biomarkers are effective in activating NK cells. Therefore, the present invention provides compositions and methods to activate and expand large numbers of NK cells in vivo, in vitro, or ex vivo for use in immunotherapeutic strategies. Such compositions, methods, and/or the activated NK cells using said compositions and methods can be employed in treatment of patients in need of increased immune response (e.g., cancer patients).

For example, one aspect of the present disclosure provides a cancer vaccine comprising cancer cells, wherein the cancer cells have a decreased copy number, amount, and/or activity of at least one biomarker that is a marker of differentiation of the cancer cells.

Another aspect provides a cancer vaccine comprising cancer cells, wherein the cancer cells have a decreased copy number, amount, and/or activity of at least one biomarker selected from Cox2, Rag2, NFkB, STAT3, CD133, NEMO, TNF-a, DAP10, DAP12, Clc-5, MCP-1, Transglutaminase 3, Presenilin 1, Presenilin 2, Annexin-1, A20 (TNFAIP3), Galectin-1, Galectin-3, PGC-1a, LDLR, Abca1, Gprc5a, BCMO1, PAP/HIP, ApoE, IL-6, IL-8, GSK3, ADAM17, NKG2A, CD3 zeta, MCT4, c-CBL, EZH2, SHP-1, and Cbl-b.

Another aspect of the present disclosure provides a pharmaceutical composition comprising a cancer vaccine described herein.

In yet another aspect, a pharmaceutical composition is provided that comprises a monocyte and/or an osteoclast, wherein the monocyte and/or the osteoclast has a decreased copy number, amount, and/or activity of at least one biomarker selected from: Cox2, Rag2, STAT3, and TNF-a. The monocyte and/or osteoclast may be irradiated (e.g., gamma irradiated). The copy number, amount, and/or activity of at least one biomarker can be decreased by contacting the cancer cells with a small molecule inhibitor, CRISPR guide RNA (gRNA), RNA interfering agent, antisense oligonucleotide, peptide or peptidomimetic inhibitor, aptamer, antibody, and/or intrabody.

Numerous embodiments are further provided that can be applied to any aspect of the present disclosure and/or combined with any other embodiment described herein. For example, the cells in the cancer vaccine and pharmaceutical composition can be irradiated (e.g., gamma irradiated). The cells may also have a decreased copy number, amount, and/or activity of Cox2, Rag2, and/or NFkB. The copy number, amount, and/or activity of at least one biomarker can be decreased by contacting the cells with a small molecule inhibitor, CRISPR guide RNA (gRNA), RNA interfering agent, antisense oligonucleotide, peptide or peptidomimetic inhibitor, aptamer, antibody, and/or intrabody.

The cancer vaccine or pharmaceutical composition can activate an NK cell, such as a primary NK cell. The cancer vaccine or pharmaceutical composition may expand the NK cell, optionally wherein the NK cell expands to at least about 10, 11, 12, 13, 14, 15, 16, 17, 18, 19, 20, 21, 22, 23, 24, 25, or more population doublings within 4 weeks. For example, the NK cell may expand a CD8+ T cell. In some embodiments, the NK cell preferentially expands a CD8+ T cell relative to a CD4+ T cell.

The cancer vaccine or pharmaceutical composition may also enhance NK cell cytotoxicity and/or increase or promote the production, secretion, and/or function of at least one cytokine or chemokine produced by the NK cell. For example, the at least one cytokine can be IFN-γ.

The cancer vaccines and pharmaceutical compositions contemplated herein may comprise one or more additional agents capable of activating an NK cell or enhancing secretion of IFN-γ by an NK cell. For example, the one or more additional agents are selected from IL-2, anti-CD16 antibody, anti-CD3 antibody, anti-CD28 antibody, and a composition comprising at least one bacterial strain.

The cancer vaccines and pharmaceutical compositions contemplated herein may also comprise at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus. In some embodiments, the at least one bacterial strain comprises Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus. The at least one bacterial strain can be sonicated.

The cells in the cancer vaccines and/or pharmaceutical compositions described herein can be obtained or derived from one or more sources. For example, the cancer cells in the cancer vaccines can be derived from a solid or hematological cancer, a cancer cell line, or from primary cancer cells. The cancer cells may derived from multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and/or glioblastoma.

The pharmaceutical compositions described herein can further comprise a cancer vaccine described herein.

A method is also provided for preventing or treating a cancer in a subject comprising administering to the subject a cancer vaccine or pharmaceutical composition described herein and/or NK cells activated by contacting the NK cells with the cancer vaccine or the pharmaceutical composition described herein.

Another method provided herein activates NK cells in a subject in need thereof, and the method comprises administering to the subject a cancer vaccine or the pharmaceutical composition described herein and/or NK cells activated by contacting the NK cells with a cancer vaccine or pharmaceutical composition described herein.

The cancer vaccine can comprise cancer cells that derived from a cancer of the same type as the cancer treated with the cancer vaccine. In some embodiments, the cancer cells in the cancer vaccine are derived from a cancer of a different type than the cancer treated with the cancer vaccine. The cancer vaccine and/or the pharmaceutical composition is syngeneic or xenogeneic to the subject. The cancer vaccine and/or the pharmaceutical composition can be autologous, matched allogeneic, mismatched allogeneic, or congenic to the subject.

The cancer vaccine and/or the pharmaceutical composition can administered systemically or locally to the cancer. For example, the cancer vaccine and/or the pharmaceutical composition is administered by intravenous, intratumoral, intramuscular, or subcutaneous administration. Additionally, the cancer vaccine and/or the pharmaceutical composition can be administered to the subject conjointly with an immunotherapy and/or cancer therapy, optionally wherein the immunotherapy and/or cancer therapy is administered before, after, or concurrently with the cancer vaccine. In some embodiments, the immunotherapy inhibits an immune checkpoint, which may be selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

The cancer therapy that can be administered before, after, or concurrently with the cancer vaccine can be radiation, a radiosensitizer, and/or a chemotherapy.

The cancer vaccine and/or pharmaceutical composition may be used to treat a cancer that is a solid or hematological cancer, multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and glioblastoma.

The cancer vaccine and/or the pharmaceutical composition may be administered at least twice to the subject, optionally wherein the cancer vaccine and/or the pharmaceutical composition are administered to the subject after at least one month since the first administration. The subject can be a mammal, optionally wherein the mammal is a mouse or human.

The methods provided herein can be part of an adjuvant therapy. The subject may be afflicted with a cancer. The cancer cells in the cancer vaccine can be derived from a cancer of the same type or a different type as the cancer treated with the cancer vaccine. The cancer vaccine and/or the pharmaceutical composition may be syngeneic or xenogeneic to the subject. The cancer vaccine and/or the pharmaceutical composition may be autologous, matched allogeneic, mismatched allogeneic, or congenic to the subject. The NK cell can be a primary NK cell. The cancer vaccine and/or the pharmaceutical composition can expand the NK cell, optionally wherein the NK cell expands to at least about 10, 11, 12, 13, 14, 15, 16, 17, 18, 19, 20, 21, 22, 23, 24, 25, or more population doublings within 4 weeks. The NK cell may expand a CD8+ T cell and preferentially expands the CD8+ T cell relative to a CD4+ T cell. The cancer vaccine and/or the pharmaceutical composition may enhance NK cell cytotoxicity and/or increases or promotes production, secretion, and/or function of at least one cytokine or chemokine produced by the NK cell. In some embodiments, the at least one cytokine is IFN-γ.

The cancer vaccine and/or the pharmaceutical composition can be administered systemically or locally to the cancer. The cancer vaccine and/or the pharmaceutical composition may be administered by intravenous, intratumoral, intramuscular, or subcutaneous administration. The cancer vaccine and/or the pharmaceutical composition can be administered to the subject conjointly with an immunotherapy and/or cancer therapy, optionally wherein the immunotherapy and/or cancer therapy is administered before, after, or concurrently with the cancer vaccine. The immunotherapy inhibits an immune checkpoint, and the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, TLT-2, ILT-4, TIGIT, HHLA2, butyrophilins, and A2aR. The cancer therapy may be selected from radiation, a radiosensitizer, and a chemotherapy. The cancer can be a solid or hematological cancer, multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, or glioblastoma.

The cancer vaccine and/or the pharmaceutical composition can administered at least twice to the subject, optionally wherein the cancer vaccine and/or the pharmaceutical composition are administered to the subject after at least one month since the first administration. The subject is a mammal, optionally wherein the mammal is a mouse or human.

In another aspect, methods are provided for activating an NK cell in vitro or ex vivo comprising contacting the NK cell with a cancer vaccine and/or the pharmaceutical composition described herein. The NK cell is a primary NK cell. The cancer vaccine and/or the pharmaceutical composition can be syngeneic or xenogeneic to the NK cell. In some embodiments, the cancer vaccine and/or the pharmaceutical composition is autologous, matched allogeneic, mismatched allogeneic, or congenic to the NK cell. The cancer vaccine and/or the pharmaceutical composition may expand the NK cell, optionally wherein the NK cell expands to at least about 10, 11, 12, 13, 14, 15, 16, 17, 18, 19, 20, 21, 22, 23, 24, 25, or more population doublings within 4 weeks. The NK cell may expand a CD8+ T cell. In some embodiments, the NK cell preferentially expands the CD8+ T cell relative to a CD4+ T cell.

The cancer vaccine and/or the pharmaceutical composition may enhance NK cell cytotoxicity and/or increase or promote production, secretion, and/or function of at least one cytokine (e.g., IFN-γ) or chemokine produced by the NK cell. The NK cell can be a mammalian cell, and optionally the mammalian cell is a mouse cell or a human cell.

Another aspect of the present invention provides NK cells activated using a method described herein for activing NK cells in vitro or ex vivo.

A pharmaceutical composition comprising the NK cells activated using a method described herein for activing NK cells in vitro or ex vivo.

In some embodiments, one or more biomarkers can be knocked down in tumor cells (e.g., patient tumor cells) as described herein to increase activation of various immune cell types. The tumor cells can be irradiated before providing them to the patient, e.g., systemically or by injection directly into the tumor of the patient. Such engineered tumor cells serve as an effective cancer vaccine, and can be an adjuvant treatment with super-charged NK cells to internally activate the patient NK cells and keep super-charged NK cells activated.

### BRIEF DESCRIPTION OF THE DRAWINGS

FIG. 1A-FIG. 1C show the increased cytotoxicity by NK cells derived from Cox-2flox/flox;LysMCre/+mice compared to those obtained from control WT littermates. Purified NK cells obtained from either control (WT) or Cox-2flox/flox;LysMCre/+ (KO) mice were left untreated or treated with IL-2 (1×10⁴ U/million) for 7 days before they were used against YAC-1 cells (FIG. 1A), Mouse Embryonic Fibroblasts (FIG. 1), and ST63 (FIG. 1C) in a standard 4 h ⁵¹Chromium release assay. The lytic units 30/10⁶ cells were determined using inverse number of NK cells required to lyse 30% of the target cells×100. *P<0.05 was obtained for the difference between control WT Cox-2flox/flox; LysMCre/+ NK cell cytotoxicity against YAC-1 cells, MEFs, or ST63. One of several representative experiments is shown in this figure.

FIG. 2A-FIG. 2B show that IL-2 activated NK cells from Cox-2flox/flox;LysMCre/+ mice cultured with autologous monocytes lysed YAC-1 cells and secreted high levels of IFN-γ as compared to NK cells from control littermates in the presence and absence of autologous monocytes. NK cells obtained from control mice or Cox-2flox/flox; LysMCre/+mice were left untreated or treated with IL-2 (1×10⁴ U/million) in the presence or absence of autologous monocytes for 7 days. Afterward, the cytotoxic function of NK cells against YAC-1 cells was determined using a standard 4 h ⁵¹Chromium release assay. *P<0.05 was obtained for the difference in cytotoxicity against YAC-1 tumors mediated by IL-2-treated NK cells cultured with or without monocytes between control and Cox-2flox/flox;LysMCre/+ mice (FIG. 2A). NK cells were treated as described in (FIG. 2A) Afterward, the supernatants were removed from the co-cultures and the levels of IFN-γ secretion were determined using specific ELISAs (FIG. 2B). *P<0.05 was obtained for the difference in IFN-γ secretion from IL-2-treated NK cells between control and Cox-2flox/flox;LysMCre/+ mice cultured with monocytes. One of several representative experiments is shown in this figure.

FIG. 3A-FIG. 3D show that monocytes, and not T cells, from Cox-2flox/flox;LysMCre/+ mice enhanced the cytotoxic function of autologous NK cells and induced high levels of IFN-γ secretion. Wild type or Cox-2flox/flox;LysMCre/+ derived NK cells were activated with IL-2 (1×10⁴ U/million) and cultured with either wild type or Cox-2flox/flox;LysMCre/+ monocytes for 7 days. Afterward, the cytotoxic function of NK cells against YAC-1 was determined using a standard 4 h ⁵¹Chromium release assay. The lytic units 30/10⁶ cells were determined using inverse number of NK cells required to lyse 30% of the target cells×100. *P<0.05 is for the difference in cytotoxicity against YAC-1 tumors between IL-2-treated NK cells from control and Cox-2flox/flox;LysMCre/+ mice cultured with monocytes (FIG. 3A). NK cells were prepared as described in (FIG. 3A) and then supernatants from NK cell cultures were harvested after co-incubation with monocytes for 7 days. Monocytes from wild type and Cox-2flox/flox;LysMCre/+ mice were used as control. The levels of IFN-γ secretion were determined using specific ELISAs. *P<0.05 is for the difference in IFN-γ secretion between IL-2-treated NK cells from control and Cox-2flox/flox; LysMCre/+ mice cultured with monocytes (FIG. 3B). NK cells were treated with IL-2 (1×10⁴ U/million) and cultured with either T cells from global COX-2 knockout mice or monocytes from wild type or Cox-2flox/flox;LysMCre/+ mice for 7 days. Afterward, NK cells were used as effectors against wild type MEFs or MEFs with specific COX-2 deletion. The cytotoxic function of NK cells against MEFs was determined using a standard 4 h ⁵¹CR release assay. The lytic units 30/10⁶ cells were determined using inverse number of NK cells required to lyse 30% of the target cells×100. *P<0.05 is for the difference in cytotoxicity between IL-2-treated NK cells from control and Cox-2flox/flox;LysMCre/+ mice cultured with monocytes or T cells (FIG. 3C). IL-2-treated (1×10⁴ U/million) NK cells obtained from wild type mice were cultured with monocytes from wild type mice or Cox-2flox/flox;LysMCre/+ mice for 7 days before the cells were used as effector cells in a standard 4 h ⁵¹Chromium release assay. Monocyte-derived DCs from wild type or Cox-2flox/flox;LysMCre/+ mice were prepared as described in Section “Materials and Methods” and used as target cells. The lytic units 30/10⁶ cells were determined using inverse number of NK cells required to lyse 30% of the target cells×100. *P<0.05 was obtained for the difference in IL-2-treated NK cell-mediated lysis between DCs from control mice and from those of Cox-2flox/flox;LysMCre/+ mice (FIG. 3D). One of several representative experiments is shown in this figure.

FIG. 4A-FIG. 4C show that the addition of LPS to NK cells cultured with monocytes induced split anergy in NK cells which resulted in significant inhibition of NK cell cytotoxicity but increased IFN-γ secretion. IL-2-treated (1×10⁴ U/million) NK cells obtained from wild type mice were cultured with monocytes from wild type mice or Cox-2flox/flox;LysMCre/+ mice for 7 days and then treated with or without LPS (20 ng/mL) for an additional day. Afterward, NK cells were used as effector cells in a standard 4 h ⁵¹Chromium release assay against YAC-1 cells. The lytic units 30/10⁶ cells were determined using inverse number of NK cells required to lyse 30% of the target cells×100. *P<0.05 was obtained for differences in cytotoxicity between untreated and LPS-treated NK cells cultured with monocytes from control littermates or those from Cox-2flox/flox; LysMCre/+ mice (FIG. 4A). NK cells were treated as described in (FIG. 4A) and afterward the supernatant was removed from the co-cultures and the levels of IFN-γ secretion were determined using specific ELISAs. *P<0.05 was obtained for differences in secretion of IFN-γ between untreated and LPS-treated NK cells cultured with monocytes from control littermates or those from Cox-2flox/flox;LysMCre/+ mice (FIG. 4B). NK cells were prepared as described in (FIG. 4A) and used as effector cells against DCs derived from monocytes from either wild type or Cox-2flox/flox; LysMCre/+ mice in a standard 4 h ⁵¹Chromium release assay. The lytic units 30/10⁶ cells were determined using inverse number of NK cells required to lyse 30% of the target cells×100 (FIG. 4C). One of several representative experiments is shown in this figure.

FIG. 5A-FIG. 5D show the MHC class-I, B7H1 and CD54 surface receptor analysis on wild type and COX-2 knockout MEFs. The surface expression of MHC class-I (FIG. 5A), B7H1 (FIG. 5B), and CD54 (FIG. 5C) on wild type and COX-2 knockout MEFs were assessed using staining with PE-conjugated antibodies followed by flow cytometric analysis. The surface expression of Rae-17 on wild type and COX-2 knockout MEFs was assessed using staining with PE-conjugated antibodies followed by flow cytometric analysis (FIG. 5D). Isotype control antibody was used as control. One of four experiments is shown.

FIG. 6A-FIG. 6D show the receptor analysis of purified splenic NK cells co-cultured with MEFs from wild type and COX-2 knockout mice. Purified NK cells were treated with IL-2 (10,000 U/mL) and cultured without and with monocytes (NK: monocytes, 2:1) and LPS (100 ng/mL) for 24 h. Afterward, NK cells were co-cultured with either wild type or COX-2 knockout MEFs or ST63 at 9:1 (NK cells: target ratio) for 48 h. Thereafter, the surface expression of DX5 (FIG. 6A), Ly49A (FIG. 6B), Ly49D (FIG. 6C) and NKG2D (FIG. 6D) were assessed using staining with PE-conjugated antibodies followed by flow cytometric analysis. Isotype control antibody was used as control. One of three experiments is shown.

FIG. 7A-FIG. 7B show that induction of split anergy mediated by LPS was observed in Human NK cells which resulted in a loss of their cytotoxic function but gained the ability to secrete high levels of IFN-γ, especially in the presence of autologous monocytes. Human NK cells were purified from healthy donors and were left untreated or treated with IL-2 (1000 U/mL), anti-CD16mAb (3 μg/mL), or the combination of IL-2 (1000 U/mL) and anti-CD16mAb (3 μg/mL) in the presence or absence of LPS (20 ng/mL) and autologous monocytes (NK cell:monocytes, 1:1) for 24-48 h. Afterward, the cytotoxicity against OSCSCs was assessed using a standard 4 h ⁵¹Chromium release assay. Percent cytotoxicity was obtained at different effector to target ratio and the lytic units 30/10⁶ cells were determined using inverse number of NK cells required to lyse 30% of the tumor cells×100 (FIG. 7A). NK cells were prepared as described in FIG. 5A. Monocytes were treated with IL-2 (1000 U/mL) and/or anti-CD16mAb (3 μg/mL) and LPS (20 ng/mL) for 24-48 h and used as controls. After the treatment period, the supernatants were removed from the co-cultures and the levels of IFN-γ cytokine were measured with specific ELISA (FIG. 7B). *P<0.05 was obtained for the differences in cytotoxicity and IFN-γ secretion between NK cells cultured in media and those treated with LPS, monocytes, or the combination of LPS and monocytes. One of several representative experiments is shown in this figure.

FIG. 8A-FIG. 8D show that split anergy induced by sAJ2 and monocytes also occurred in Human NK cells. Human NK cells were purified from healthy donors and left untreated or treated with IL-2 (1000 U/mL) or the combination of IL-2 (1000 U/mL) and anti-CD16mAb (3 μg/mL) in the presence of sAJ2 (NK cell:sAJ2, 1:3), autologous monocytes (NK cell:monocytes, 1:1) or the combination of sAJ2 (NK cell:sAJ2, 1:3) and autologous monocytes (NK cell:monocytes, 1:1) for 24-48 h. Afterward, the cytotoxicity against OSCSCs cells was assessed using a standard 4 h ⁵¹Chromium release assay. Percent cytotoxicity was obtained at different effector to target ratio and the lytic units 30/10⁶ cells were determined using inverse number of NK cells required to lyse 30% of the tumor cells×100 (FIG. 8A). NK cells were prepared as described in FIG. 6A and after the treatment period, the supernatants were removed from the co-cultures and the levels of IFN-γ cytokine were measured with specific ELISA. *P<0.05 was obtained for the differences in cytotoxicity and IFN-γ secretion between human NK cells cultured in media and those treated with sAJ2, monocytes or the combination of sAJ2 and monocytes. One of several representative experiments is shown in this figure (FIG. 8B). Purified NK cells were cultured with autologous monocytes (NK cell:monocytes, 1:1). After an overnight incubation, the supernatants were collected and the levels of IFN-γ, IL-15, IFN-α, and IL-12 were determined by ELISAs in a multiplexed format using Luminex technology (FIG. 8C). Untreated and IL-2 stimulated NK cells were treated with monocytes and sAJ2 as described in (FIG. 8A). Afterward, the supernatants were collected and the level of IFN-α was determined by ELISAs in a multiplexed format using Luminex technology (FIG. 8D).

FIG. 9A-FIG. 9C show histological examination of the tooth-extraction wound socket at 4 weeks after maxillary first molar extraction. Alveolar bone regeneration in the tooth extraction socket of wild type (Bulb/c), Rag2−/− and Rag2−/−γc−/− mice. New bone regeneration (N.B.) was observed inside (dotted line) and below the edge (white arrowheads) of the socket (Soc) in wild type and Rag2−/− mice. By contrast, new bone regeneration of Rag2−/−γc−/− mice was observed above the socket edge of the alveolar bone (FIG. 9A). The top surface of alveolar bone interfaces the gingival barrier tissue. Inflammatory cell infiltrates (black arrows) in gingiva were localized on the top surface of alveolar bone and new bone in the socket of wild type and Rag2−/− mice. However, gingival inflammatory cells were missing in Rag2−/−γc−/− mice (FIG. 9B). Tartrate-resistant acid phosphatase (TRAP) positive osteoclasts (red staining; black arrowheads) were identified on the surface of alveolar bone interfacing gingival barrier tissue as well as within the new bone formed inside the socket in wild type mice. In Rag2−/− mice, TRAP-positive osteoclasts were observed in the new bone but not on the alveolar bone surface. TRAP-positive osteoclasts were not observed on the surface and within the new bone formed in the socket of Rag2−/−γc−/− mice (FIG. 9C).

FIG. 10A-FIG. 10K show that increased levels of bone remodeling activity is seen in Rag2−/− and Rag2−/−γc−/− mice in comparison to WT mice. Two sequential IP injections of Calcein (12 mg/ml) at 5 days apart were performed in 8 weeks old female B6 (n=3), Rag2−/− (n=3) and Rag2−/−γc−/− (n=3) mice. Two days after the second Calcein injection, mice were euthanized and L3 lumber bones were harvested. L3 bones were fixed in 70% EtOH at 4° C. in a dark place. L3 bones were prepared for non-decalcified plastic section for a conventional bone morphometric analysis (FIG. 10A-FIG. 10K).

FIG. 11A-FIG. 11B show the Decreased total cell counts but within total cells increased percentages of NK cells and monocytes in the BM of Rag2^{-/-} mice in comparison to BM of WT mice. BM cells were isolated from WT and RAG2 KO mice femur as described in Materials and Method section, freshly isolated BM cells were counted (n=9) (FIG. 11A). Surface expression of mouse Nkp46, DX5, F4/80, CD3 was determined using antibody staining followed by flow cytometric analysis as described in Materials and Methods section (n=9) (FIG. 11B).

FIG. 12A-FIG. 12C show the Decreased total cell counts but within total cells increased percentages of NK cells and monocytes in the spleen of Rag2^{-/-} mice in comparison to spleen of WT mice. Splenocytes were isolated from WT and RAG2 KO mice as described in Materials and Methods section, freshly isolated splenocytes were counted (n=9) (FIG. 12A). Surface expression of mouse Nkp46, DX5, F4/80, CD3 were determined using antibody staining followed by flow cytometric analysis as described in Materials and Methods section (n=9) (FIG. 12B). NK cells were sorted out from WT and RAG2 KO mice total splenocytes as described in Materials and Methods section, freshly purified NK cells were counted (n=9) (FIG. 12C).

FIG. 13A-FIG. 13B show increased percentages of NK cells in the oral gingiva of Rag2^{-/-} mice in comparison to oral gingiva of WT mice. Oral gingiva were isolated from WT and RAG2 KO mice as described in Materials and Methods section, freshly isolated gingival cells were counted. Surface expression of mouse DX5 was determined using antibody staining followed by flow cytometric analysis as described in Materials and Methods section (n=3) (FIG. 13A). One of the representative flow panel is shown in the figure (FIG. 13B).

FIG. 14 shows the increased IFN-γ secretion by various tissue compartments in Rag2^{-/-} mice in comparison to WT mice when similar numbers of cells were cultured. BM cells (n=9), splenocytes (n=9), pancreas (n=5), oral gingiva (n=5), peri-pancreatic adipose tissue (n=5), NK cells purified from splenocytes (n=5) of WT and RAG2 KO mice were cultured at (2×10⁶ cells/2 ml) each treated with IL-2 (10000 U/ml) for 5 days, after which the supernatants were harvested and the levels of IFN-γ were determined using specific ELISAs.

FIG. 15 shows the increased NK cell-mediated cytotoxicity by BM cells, splenocytes and NK cells in Rag2^{-/-} mice in comparison to WT mice when similar numbers of effector cells were used. BM cells (n=9), splenocytes (n=9), NK cells purified from splenocytes (n=5), pancreas (n=5), peri-pancreatic adipose tissue (n=5), of WT and RAG2 KO mice were cultured at (2×10⁶ cells/2 ml) each treated with IL-2 (10000 U/ml) for 5 days. After which cytotoxicity assays were performed using standard 4-hour ⁵¹Cr release assay using similar number of effector cells against st63 cells. LU 30/10⁶ cells were determined using inverse number of cells required to lyse 30% of st63 cells×100.

FIG. 16A-FIG. 16B show that ZOL induced increased IFN-γ secretion and NK cell-mediated cytotoxicity by BM cells but reverse effect was seen in splenocytes, NK cells and oral gingiva cells in Rag2^{-/-} mice. RAG2 mice were administered with either 0.9% NaCl or ZOL (500 μg/kg) via tail vein followed by maxillary left first molar extraction as described in Materials and Methods section. Week 4 after the tooth extraction, animals were sacrificed, BM cells (n=9), splenocytes (n=9), NK cells purified from splenocytes (n=5), and oral gingiva (n=5) cells were cultured at (1×10⁶ cells/ml) each treated with IL-2 (10000 U/ml) for 5 days, after which the supernatants were harvested and the levels of IFN-γ were determined using specific ELISAs (FIG. 16A). Week 4 after the tooth extraction, animals were sacrificed, BM cells (n=9), splenocytes (n=9), and NK cells purified from splenocytes (n=5) were cultured at (1×10⁶ cells/ml) each treated with IL-2 (10000 U/ml) for 5 days, after which the cells were used as effector cells against tumor targets. Cytotoxicity assays were performed using standard 4-hour ⁵¹Cr release assay against st63 cells, and the LU 30/10⁶ cells were determined using inverse number of cells required to lyse 30% of st63 cells×100 (FIG. 16B).

FIG. 17A-FIG. 17B show that treatment of vector-alone transfected HEp2 cells with Sulindac increased cytotoxicity and cytokine secretion by NK cells. (FIG. 17A) 4×10⁶ vector-alone transfected HEp2 cells were labeled with ⁵¹Cr for 1 hour at 37° C. The cells were then washed three times to remove the excess ⁵¹Cr, and they were left untreated or treated with 50-200 μm of Sulindac for 3-4 hours. Untreated and Sulindac treated ⁵¹Cr labeled vector-alone transfected HEp2 cells were added to untreated (solid lines) and IL-2 (500 u/ml) treated (dashed lines) NK cells. Treatment of NK cells with IL-2 was carried out for 8-12 hours before their addition to the target cells. (FIG. 17B) Vector-alone transfected HEp2 cells were left untreated or treated with Sulindac (200 μm), Adriamycin (10 μg/ml) and DMSO. After an overnight incubation the cells were washed to remove the drugs, and the tumor cells were then co-cultured in the presence of untreated and IL-2 (500 u/ml) treated NK cells. Treatment of NK cells with IL-2 was carried out for 8-12 hours before their addition to the target cells. After 48 hours of incubation the supernatants were removed from the NK-HEp2 cell co-cultures and assayed for released GM-CSF.

### DETAILED DESCRIPTION OF THE INVENTION

It has been determined herein that a cancer vaccine comprising cancer cells deficient in one or more biomarkers are effective in activating NK cells and inducing immune response. Such deficiency results in de-differentiation of the cancer cells, and the proteins presented on their cell surface provide effective signals in activating various types of immune cells, including NK cells. Similarly, it has been determined herein that a monocyte and/or an osteoclast deficient in one or more biomarkers are effective in activating NK cells. Therefore, the present invention provides compositions and methods to activate and expand large numbers of NK cells in vivo, in vitro, or ex vivo for use in immunotherapeutic strategies. Such compositions, methods, and/or the activated NK cells using said compositions and methods can be employed in treatment of patients in need of increased immune response (e.g., cancer patients).

In some embodiments, one or more biomarkers can be knocked down in tumor cells (e.g., patient tumor cells) as described herein to increase activation of various immune cell types. The tumor cells can be irradiated before providing them to the patient, e.g., systemically or by injection directly in the tumor of the patient. Such engineered tumor cells serve as an effective cancer vaccine, and can be an adjuvant treatment with super-charged NK cells to internally activate the patient NK cells and keep super-charged NK cells activated.

In some embodiments, provided herein are cancer vaccines comprising cancer cells that have at least, about, or no more than 5%, 10%, 15%, 20%, 25%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 8100, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% decrease in a copy number, amount, and/or activity of at least one biomarker that is a marker of differentiation of the cancer cells (e.g., biomarkers described herein) (e.g., as compared with a control).

In some embodiments, provided herein are cancer vaccines comprising cancer cells that have at least about 5%, 10%, 15%, 20%, 25%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% decrease in a copy number, amount, and/or activity of at least one biomarker selected from: Cox2, Rag2, NFkB, STAT3, CD133, NEMO, TNF-a, DAP10, DAP12, Clc-5, MCP-1, Transglutaminase 3, Presenilin 1, Presenilin 2, Annexin-1, A20 (TNFAIP3), Galectin-1, Galectin-3, PGC-1a, LDLR, Abca1, Gprc5a, BCMO1, PAP/HIP, ApoE, IL-6, IL-8, GSK3, ADAM17, NKG2A, CD3 zeta, MCT4, c-CBL, EZH2, SUP-1, and Cbl-b (e.g., as compared with a control).

In certain embodiments, the percent decrease in the copy number, amount, and/or activity of at least one biomarker is proportionally correlated with the activation of the immune response (e.g., activation of NK cells). For example, a 25% decrease in the amount and/or activity of the biomarker of the present disclosure in cancer cells may result in a 25% increase in the NK cell activation.

In some embodiments, provided herein are pharmaceutical compositions comprising a monocyte and/or an osteoclast, wherein the monocyte and/or the osteoclast has at least about 5%, 10%, 15%, 20%, 25%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% decrease in copy number, amount, and/or activity of at least one biomarker selected from: Cox2, STAT3, Rag2, and TNF-a.

### Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent to perform its intended function. Examples of routes of administration for treatment of a body which can be used include injection (subcutaneous, intravenous, parenteral, intraperitoneal, intrathecal, etc.), oral, inhalation, and transdermal routes. The injections can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

The term “altered copy number” refers to increased or decreased copy number (e.g., germline and/or somatic) of a biomarker DNA as compared to the copy number of the biomarker DNA in a control sample. The term “altered amount” of a biomarker includes an increased or decreased RNA level or protein level of a biomarker in a sample, e.g., a cancer sample, as compared to the corresponding protein level in a normal, control sample. Similarly, the term “altered activity” of a biomarker includes an increased or decreased activity of the biomarker protein in a sample as compared to the corresponding activity in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors. An altered amount or activity of a biomarker protein may be determined by detecting posttranslational modification such as phosphorylation status of the marker, which may affect the expression or activity of the biomarker protein. An altered amount or activity of a biomarker protein may be due to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

The term “control” refers to any reference standard suitable to provide a comparison to the expression products in the test sample. Such a control may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In other embodiments, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In some embodiments, the control may comprise normal or non-cancerous cell/tissue sample. In other embodiments, the control may comprise an expression level for a set of patients, such as a set of cancer patients, or for a set of cancer patients receiving a certain treatment, or for a set of patients with one outcome versus another outcome. In other preferred embodiments, the control may comprise normal cells, cells from patients treated with combination chemotherapy, and cells from patients having benign cancer. In still other embodiments, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In other embodiments, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample.

The amount of a biomarker in a cell is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the cell can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The term “subject” or “patient” refers to any healthy or diseased animal, mammal or human, or any animal, mammal or human. In some embodiments, the subject is afflicted with cancer. In some embodiments, the subject is in need of an increased immune response. In some embodiments, the subject is in need of an increased NK cell activity. In various embodiments of the methods of the present invention, the subject has not undergone treatment, such as chemotherapy, radiation therapy, targeted therapy, and/or immunotherapies. In other embodiments, the subject has undergone treatment, such as chemotherapy, radiation therapy, targeted therapy, and/or immunotherapies. In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

A “therapeutically effective amount” of a substance or cells is an amount capable of producing a medically desirable result in a treated patient, e.g., decrease tumor burden, decrease the growth of tumor cells, or alleviate any symptom associated with cancer, with an acceptable benefit: risk ratio, preferably in a human or non-human mammal.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof).

### Biomarkers

As described herein, many biomarkers are important for differentiation of cancer cells. Reducing the copy number, amount, and/or activity of one or more such biomarkers in cancer cells results in de-differentiation of the cancer cells. Cancer cells, monocytes, and/or osteoclasts comprising a decreased copy number, amount, and/or activity of the biomarkers described herein are particularly effective in inducing immune response and activate immune cells (e.g., NK cells), thus they provide novel strategy in treating patients in need of increased immune response (e.g., cancer patients). Representative biomarkers are described in detail below, although many other suitable markers can be used instead of or in addition to one or more of these.

In certain aspects, the biomarker of the present disclosure is selected from Cox2, Rag2, NFkB, STAT3, CD133, NEMO, TNF-a, DAP10, DAP12, Clc-5, MCP-1, Transglutaminase 3, Presenilin 1, Presenilin 2, Annexin-1, A20 (TNFAIP3), Galectin-1, Galectin-3, PGC-1a, LDLR, Abca1, Gprc5a, BCMO1, PAP/HIP, ApoE, IL-6, IL-8, GSK3, ADAM17, NKG2A, CD3 zeta, MCT4, c-CBL, EZH2, SUP-1, and Cbl-b (see also Table 1).

Cox2 is also known as Prostaglandin-Endoperoxide Synthase 2 (PTGS2) or cyclooxygenase 2. Cox2 is the key enzyme in prostaglandin biosynthesis, and acts both as a dioxygenase and as a peroxidase. It is a dual cyclooxygenase and peroxidase in the biosynthesis pathway of prostanoids, a class of C20 oxylipins mainly derived from arachidonate, with a particular role in the inflammatory response. The cyclooxygenase activity oxygenates arachidonate (AA, C20:4(n-6)) to the hydroperoxy endoperoxide prostaglandin G2 (PGG2), and the peroxidase activity reduces PGG2 to the hydroxy endoperoxide PGH2, the precursor of all 2-series prostaglandins and thromboxanes. This complex transformation is initiated by abstraction of hydrogen at carbon 13 (with S-stereochemistry), followed by insertion of molecular O2 to form the endoperoxide bridge between carbon 9 and 11 that defines prostaglandins. The insertion of a second molecule of O2 (bis-oxygenase activity) yields a hydroperoxy group in PGG2 that is then reduced to PGH2 by two electrons. Similarly catalyzes successive cyclooxygenation and peroxidation of dihomo-gamma-linoleate (DGLA, C20:3(n-6)) and eicosapentaenoate (EPA, C20:5(n-3)) to corresponding PGH1 and PGH3, the precursors of 1- and 3-series prostaglandins. In an alternative pathway of prostanoid biosynthesis, it converts 2-arachidonoyl lysophopholipids to prostanoid lysophopholipids, which are then hydrolyzed by intracellular phospholipases to release free prostanoids. Cox2 metabolizes 2-arachidonoyl glycerol yielding the glyceryl ester of PGH2, a process that can contribute to pain response. It generates lipid mediators from n-3 and n-6 polyunsaturated fatty acids (PUFAs) via a lipoxygenase-type mechanism. It oxygenates PUFAs to hydroperoxy compounds and then reduces them to corresponding alcohols. Cox2 plays a role in the generation of resolution phase interaction products (resolvins) during both sterile and infectious inflammation. Cox2 metabolizes docosahexaenoate (DHA, C22:6(n-3)) to 17R-HDHA, a precursor of the D-series resolvins (RvDs). As a component of the biosynthetic pathway of E-series resolvins (RvEs), converts eicosapentaenoate (EPA, C20:5(n-3)) primarily to 18S-HEPE that is further metabolized by ALOX5 and LTA4H to generate 18S-RvE1 and 18S-RvE2. In vascular endothelial cells, converts docosapentaenoate (DPA, C22:5(n-3)) to 13R-HDPA, a precursor for 13-series resolvins (RvTs) shown to activate macrophage phagocytosis during bacterial infection. In activated leukocytes, contributes to oxygenation of hydroxyeicosatetraenoates (HETE) to diHETES (5,15-diHETE and 5,11-diHETE). Diseases associated with PTGS2 include Gastric Ulcer and Bursitis.

Anti-Cox2 antibodies are well known in the art and include, for example, antibodies AP00118PU-N, AP07624SU-N, AP20194PU-N, AP23344PU-N, AP53500PU-N, CF805224, CF805286, CF805287, CF805307, CF805310, CF805311, CF805365, CF805370, TA302755, TA313292, TA319280, TA326006, TA590771, TA805224, TA805224S, TA805286, TA805286S, TA805287, TA805287S, TA805307, TA805307S, TA805310, TA805310S, TA805311, TA805311S, TA805365, TA805365S, TA805370, and TA805370S (Origene); and antibodies Cox-2 (29), Cox-2 (D-12), and Cox-2 (H-3) (Santa Cruz Biotechnology). In addition, Cox-2 small molecule inhibitors are well known in the art and include, for example, rofecoxib, celecoxib, lumiracoxib, valdecoxib, diclofenac, etoricoxib, acetaminophen, ketoprofen, ketorolac, nabumentone, oxaprozin, salicyclic acid, sulindac, carprofen, mefenamic acid, parecoxib, etodolac, indomethacin, nimesulide, piroxicam, bromfenac, flurbiprofen, lornoxicam, meclofenamic acid, meloxicam, naproxen, salsalate, balsalazide, diflunisal, fenoprofen, mesalazine, nepafenac, phenylbutazone, sulfasalazine, suprofen, tiaprofenic acid, tolmetin, aceclofenac, acemetacin, tenoxicam, NS398, FK3311, saikosaponin A, and wogonin. Inhibitory RNA products are well known in the art and include, for example, COX2 (PTGS2) Human shRNA Lentiviral Particle (TL310074V), COX2 (PTGS2) Human siRNA Oligo Duplex (SR303873), Ptgs2 Mouse shRNA Plasmid (TG501784), Ptgs2 Rat shRNA Plasmid (TL710033), and Ptgs2 Mouse siRNA Oligo Duplex (SR417403) (OriGene). CRISPR products are well known in the art and include, for example, GA103907, GA203460, KN202245, KN202245BN, KN202245LP, KN202245RB, KN314183, KN314183BN, KN314183LP, KN314183RB, KN402245, and KN514183 (OriGene); and Cox-2 CRISPR Activation Plasmid (h), Cox-2 CRISPR Activation Plasmid (h2), and Cox-2 CRISPR/Cas9 KO Plasmid (h2) (Santa Cruz Biotechnology). Similarly, miRNA products are well known in the art and include HmiT107759, HmiT117890, MmiT088554, and RmiT049751 (GeneCopoeia™).

Rag2 is also known as Recombination Activating 2. Rag2 encodes a protein that is involved in the initiation of V(D)J recombination during B and T cell development. This protein forms a complex with the product of the adjacent recombination activating gene 1, and this complex can form double-strand breaks by cleaving DNA at conserved recombination signal sequences. The recombination activating gene 1 component is thought to contain most of the catalytic activity, while the N-terminal of the recombination activating gene 2 component is thought to form a six-bladed propeller in the active core that serves as a binding scaffold for the tight association of the complex with DNA. A C-terminal plant homeodomain finger-like motif in this protein is necessary for interactions with chromatin components, specifically with histone H3 that is trimethylated at lysine 4. Mutations in this gene cause Omenn syndrome, a form of severe combined immunodeficiency associated with autoimmune-like symptoms. Diseases associated with RAG2 include Omenn Syndrome and Combined Cellular And Humoral Immune Defects With Granulomas.

Anti-Rag2 antibodies are well known in the art and include, for example, AM06429SU-N, TA307957, and TA332798 (OriGene); orb167458, orb340947 orb556630 (Biorbyt); and RAG-2 (4D5C7) (Santa Cruz Biotechnology). Inhibitory RNA products are well known in the art and include, for example, RAG2 Human shRNA Lentiviral Particle (TL309958V), RAG2 Human siRNA Oligo Duplex (SR303974), RAG2 Human shRNA Plasmid Kit (TG309958), Rag2 Mouse shRNA Plasmid (TL513282), Rag2 Mouse shRNA Lentiviral Particle (TL513282V), and Rag2 Mouse siRNA Oligo Duplex (SR416756) (OriGene); and sc-141820, sc-36371, and sc-36372 (Santa Cruz Technology). CRISPR products are well known in the art and include, for example, GA104009, GA203558, KN405065, and KN514435 (OriGene); and RAG-2 CRISPR/Cas9 KO Plasmid (h), RAG-2 CRISPR Activation Plasmid (h), and RAG-2 CRISPR Activation Plasmid (h2) (Santa Cruz Biotechnology). The miRNA products are well known in the art and include, for example, HmiT059693, HmiT059694, HmiT108493, MmiT094626, and RmiT055488 (GeneCopoeia).

NFκB is a transcription factor that coordinates the activation of numerous genes in response to pathogens and proinflammatory cytokines and, therefore, is vital in the development of acute and chronic inflammatory diseases. More importantly, NFκB has been demonstrated to respond to a variety of metabolic stress signals, and protects the cells from undergoing cell death. Therefore, NFκB represents a survival factor for many tumor cells. Accordingly, NF-kappa-B is a ubiquitous transcription factor involved in several biological processes. NF-kappa-B is composed of NFKB1 or NFKB2 bound to either REL, RELA, or RELB. The most abundant form of NF-kappa-B is NFKB1 complexed with the product of this gene, RELA. Exemplar NFκB exists as a 50-kDa (p50) and a 65-kDa (p65) heterodimer. In many cells NFκB is found in the cytoplasm, where it is associated with an inhibitor protein known as IκB. NFκB is activated by phosphorylation of its inhibitory subunit, IκB-α, on serine residues 32 and 36 by cytokine-activated IκB kinases (IKKs). The inhibitor protein IκB-α maintains NFκB in an inactive form in the cytoplasms of unstimulated cells. Upon cell activation, IκB-α is rapidly degraded, leading to nuclear translocation of the free NFκB. NFκB regulates many genes, including some of the key adhesion molecules. Adhesion molecules play an integral role in tumor growth, invasion, and metastasis, and have been characterized to influence the immune responses to malignant cells.

NFkB1 is also known as Nuclear Factor NF-Kappa-B P50 Subunit. This gene encodes a 105 kD protein which can undergo cotranslational processing by the 26S proteasome to produce a 50 kD protein. The 105 kD protein is a Rel protein-specific transcription inhibitor and the 50 kD protein is a DNA binding subunit of the NF-kappa-B (NFkB) protein complex. NFkB is a transcription regulator that is activated by various intra- and extra-cellular stimuli such as cytokines, oxidant-free radicals, ultraviolet irradiation, and bacterial or viral products. Activated NFkB translocates into the nucleus and stimulates the expression of genes involved in a wide variety of biological functions. Inappropriate activation of NFkB has been associated with a number of inflammatory diseases while persistent inhibition of NFkB leads to inappropriate immune cell development or delayed cell growth. Alternative splicing results in multiple transcript variants encoding different isoforms, at least one of which is proteolytically processed.

RELA is also known as Nuclear Factor NF-Kappa-B P65 Subunit. As described above, NF-kappa-B is a homo- or heterodimeric complex formed by the Rel-like domain-containing proteins RELA/p65, RELB, NFKB1/p105, NFKB1/p50, REL and NFKB2/p52. The heterodimeric RELA-NFKB1 complex appears to be most abundant one. The dimers bind at kappa-B sites in the DNA of their target genes and the individual dimers have distinct preferences for different kappa-B sites that they can bind with distinguishable affinity and specificity. Different dimer combinations act as transcriptional activators or repressors, respectively. The NF-kappa-B heterodimeric RELA-NFKB1 and RELA-REL complexes, for instance, function as transcriptional activators. NF-kappa-B is controlled by various mechanisms of post-translational modification and subcellular compartmentalization as well as by interactions with other cofactors or corepressors. NF-kappa-B complexes are held in the cytoplasm in an inactive state complexed with members of the NF-kappa-B inhibitor (I-kappa-B) family. In a conventional activation pathway, I-kappa-B is phosphorylated by I-kappa-B kinases (IKKs) in response to different activators, subsequently degraded thus liberating the active NF-kappa-B complex which translocates to the nucleus. The inhibitory effect of I-kappa-B on NF-kappa-B through retention in the cytoplasm is exerted primarily through the interaction with RELA. RELA shows a weak DNA-binding site which could contribute directly to DNA binding in the NF-kappa-B complex. Beside its activity as a direct transcriptional activator, it is also able to modulate promoters accessibility to transcription factors and thereby indirectly regulate gene expression. Associates with chromatin at the NF-kappa-B promoter region via association with DDX1. Essential for cytokine gene expression in T-cells. The NF-kappa-B homodimeric RELA-RELA complex appears to be involved in invasin-mediated activation of TL-8 expression. Diseases associated with RELA include Mucocutaneous Ulceration, Chronic and Rela Fusion-Positive Ependymoma.

Anti-NF-kB antibodies are well known in the art and include, for example, Inhibitors of NF-kB are well known in the art and include, for example, dimethyl fumarate, gefitinib, guanosine 5′-diphosphate 2′:3′-cyclic monophosphate, guanosine diphosphate, SC236, JSH-23, Kaempferol, matrine, QNZ (EVP4593), thalidomide, donepezil, glycyrrhizin, pseudoephedrine, triflusal, Pyrrolidinedithiocarbamate ammonium, SP100030, tanshinone IIA, andrographolide, bardoxolone, Bay 11-7085, Bay 11-7821 (Bay 11-7082), and triptolide. As used herein, polypeptide inhibitors of NF-kB are well known in the art, including a construct comprising IκB(S32AS36A). For example, the pRcCMV-IκB(S32AS36A) construct contains the mutant IκB that inhibits NFκB. Specifically, the mutant IκB contains substitutions of alanine at the serines 32 and 36, thus the designation of (S32AS36A) for this vector. Substitution of alanines for serines inhibits phosphorylation and subsequent degradation of IκB. Consequently, NFκB remains bound to IκB in the cytoplasm and is unable to move to the nucleus. Inhibitory RNA products are well known in the art and include, for example, NF-kB p65 (RELA) Human siRNA Oligo Duplex (SR321602), NF-kB p65 (RELA) Human shRNA Plasmid Kit (TR302038), Rela Mouse shRNA Plasmid (TR516660), NF-kB p65 (RELA) Human shRNA Plasmid Kit (TG302038), NF-kB p65 (RELA) Human shRNA Plasmid Kit (TL302038), Rela Mouse siRNA Oligo Duplex (SR427138), Rela Mouse shRNA Plasmid (TF516660), Rela Mouse shRNA Plasmid (TL516660), NF-kB p65 (RELA) Human shRNA Lentiviral Particle (TL302038V), NFKB1 Human siRNA Oligo Duplex (SR321103), NFKB1 Human shRNA Plasmid Kit (TR318700), Nfkb1 Mouse shRNA Plasmid (TR516641), NFKB1 Human shRNA Lentiviral Particle (TL318700V), and Nfkb1 Mouse shRNA Lentiviral Particle (TL516641V) (OriGene). CRISPR reagents are well known in the art and include, for example, GA103192, GA202883, KN208384, KN208384BN, KN208384LP, KN208384RB, KN310944, KN310944BN, KN310944LP, KN310944RB, KN408384, and KN510944 (OriGene); and NFκB p50 CRISPR Activation Plasmid (h), NFκB p50 CRISPR Activation Plasmid (h2), NFκB p50 CRISPR/Cas9 KO Plasmid (h2), NFκB p65 CRISPR Activation Plasmid (h), NFκB p65 CRISPR Activation Plasmid (h2), and NFκB p65 CRISPR/Cas9 KO Plasmid (h2).

STAT3 is also known as Signal Transducer And Activator Of Transcription 3. STAT3 is a member of the STAT protein family. In response to cytokines and growth factors, STAT family members are phosphorylated by the receptor associated kinases, and then form homo- or heterodimers that translocate to the cell nucleus where they act as transcription activators. This protein is activated through phosphorylation in response to various cytokines and growth factors including IFNs, EGF, IL5, IL6, HGF, LIF and BMP2. This protein mediates the expression of a variety of genes in response to cell stimuli, and thus plays a key role in many cellular processes such as cell growth and apoptosis. The small GTPase Rac1 has been shown to bind and regulate the activity of this protein. PIAS3 protein is a specific inhibitor of this protein. Mutations in this gene are associated with infantile-onset multisystem autoimmune disease and hyper-immunoglobulin E syndrome. Alternative splicing results in multiple transcript variants encoding distinct isoforms. Diseases associated with STAT3 include Hyper-Ige Recurrent Infection Syndrome 1, Autosomal Dominant and Autoimmune Disease, Multisystem, Infantile-Onset, 1.

Anti-STAT3 antibodies are well known in the art and include, for example, STAT3 Phospho (Tyr705) Antibody (651001), STAT3 Phospho (Tyr705) Antibody (651002), STAT3 Antibody (678002), STAT3 Antibody (678003), STAT3 Phospho (Tyr705) Antibody (651003), STAT3 Phospho (Tyr705) Antibody (651004), STAT3 Phospho (Tyr705) Antibody (651010), STAT3 Phospho (Tyr705) Antibody (651008), STAT3 Phospho (Tyr705) Antibody (651005), STAT3 Phospho (Tyr705) Antibody (651007), STAT3 Phospho (Tyr705) Antibody (651009), and STAT3 Phospho (Tyr705) Antibody (651006) (BioLegend). STAT3 inhibitors known in the art include niclosamide, amphotericin B, clotrimazone, cryptotanshinone, napabucasin, SD 1008, corylifol A, cucurbitacin I, FLLL32, and HO-3867. Inhibitory RNA reagents for STAT3 include, for example, STAT3 Human shRNA Plasmid Kit (TR301348), STAT3 Human siRNA Oligo Duplex (SR321907), STAT3 Human shRNA Plasmid Kit (TL301348), STAT3 Human shRNA Plasmid Kit (TF301348), STAT3 Human shRNA Plasmid Kit (TG301348), Stat3 Rat shRNA Plasmid (TG709368), and STAT3 Human shRNA Lentiviral Particle (TL301348V) (OriGene). CRISPR reagents are well known in the art and include, for example, GA104661, GA204124, KN204922, KN204922BN, KN204922LP, KN204922RB, KN316845, KN316845BN, KN316845LP, KN316845RB, KN404922, and KN516845 (OriGene).

CD133 is also known as PROM1 or Prominin 1. CD133 is a pentaspan transmembrane glycoprotein. The protein localizes to membrane protrusions and is often expressed on adult stem cells, where it is thought to function in maintaining stem cell properties by suppressing differentiation. Mutations in this gene have been shown to result in retinitis pigmentosa, macular dystrophy, and Stargardt disease. Expression of this gene is also associated with several types of cancer. This gene is expressed from at least five alternative promoters that are expressed in a tissue-dependent manner. Multiple transcript variants encoding different isoforms have been found for this gene.

Anti-CD133 antibodies are well known in the art and include, for example, TA309943, TA326777, TA347032, TA347033, TA349098, TA350943, TA354470, and TA354471 (OriGene). Inhibitory RNA reagents are well known in the art and include, for example, CD133 (PROM1) Human siRNA Oligo Duplex (SR305838), CD133 (PROM1) Human shRNA Plasmid Kit (TG310163), Prom1 Mouse shRNA Plasmid (TL501745), Prom1 Mouse shRNA Lentiviral Particle (TL501745V), Prom1 Mouse siRNA Oligo Duplex (SR420593), Prom1 Rat shRNA Lentiviral Particle (TL710503V), and CD133 (PROM1) Human shRNA Lentiviral Particle (TL310163V) (OriGene). CRISPR reagents are well known in the art and include, for example, GA105884, GA203402, KN221611, KN221611BN, KN221611LP, KN221611RB, KN421611, and KN513953 (OriGene).

NEMO is also known as Inhibitor Of Nuclear Factor Kappa B Kinase Regulatory Subunit Gamma (IKBKG). NEMO is a regulatory subunit of the inhibitor of kappaB kinase (IKK) complex, which activates NF-kappaB resulting in activation of genes involved in inflammation, immunity, cell survival, and other pathways. Mutations in this gene result in incontinentia pigmenti, hypohidrotic ectodermal dysplasia, and several other types of immunodeficiencies.

Anti-NEMO antibodies are well known in the art and include, for example, IKKgamma (NEMO) Antibody (694101) and IKKgamma (NEMO) Antibody (694102) (BioLegend); and orb390140, orb393327, orb125366, orb382565, orb48414, orb146377, orb214796, orb214797, and orb214798 (Biorbyt). Small molecule inhibitors of NEMO are well known in the art and include, for example, artesunate, primaquine, pyrimethamine, sulfadoxine, and luteolin. Inhibitory RNA reagents are well known in the art and include, for example, Ikbkg Mouse siRNA Oligo Duplex (SR413644), Ikbkg Rat shRNA Plasmid (TF713726), IKK gamma (IKBKG) Human shRNA Lentiviral Particle (TL312203V), IKK gamma (IKBKG) Human shRNA Plasmid Kit (TL312203), and IKK gamma (IKBKG) Human siRNA Oligo Duplex (SR322432) (OriGene). CRISPR reagents are well known in the art and include, for example, GA105633, GA202111, KN201743, KN201743BN, KN201743LP, KN201743RB, KN401743, and KN508212 (OriGene).

TNF-α is also known as tumor necrosis factor or tumor necrosis factor-alpha. TNF-α is a multifunctional proinflammatory cytokine that belongs to the tumor necrosis factor (TNF) superfamily. This cytokine is mainly secreted by macrophages. It can bind to, and thus functions through its receptors TNFRSF1A/TNFR1 and TNFRSF1B/TNFBR. This cytokine is involved in the regulation of a wide spectrum of biological processes including cell proliferation, differentiation, apoptosis, lipid metabolism, and coagulation. This cytokine has been implicated in a variety of diseases, including autoimmune diseases, insulin resistance, and cancer. Knockout studies in mice also suggested the neuroprotective function of this cytokine.

Anti-TNF-α antibodies are well known in the art and include, for example, orb313248, orb318721, orb323199, orb475251, orb11494, orb11495, orb7100, orb345126, orb345125, orb128850, orb129753, orb371962, orb376634, orb385905, orb388725, orb388731, orb388736, orb12524, orb239747, and orb214681 (Biorbyt). Small molecule and biologic inhibitors of TNF-α are well known in the art and include, for example, adalimumab, etanercept, infliximab, certolizumab pegol, thalidomide, Pyrrolidinedithiocarbamate ammonium, SP 100030, calcitriol, Bay 11-7821 (Bay 11-7082), cepharanthine, and lenalidomide. Inhibitory RNA reagents are well known in the art and include, for example, TNF alpha (TNF) Human shRNA Plasmid Kit (TG318768), Tnf Mouse shRNA Plasmid (TL515379), Tnf Mouse siRNA Oligo Duplex (SR406508), Tnf Rat shRNA Lentiviral Particle (TL709307V), and TNF alpha (TNF) Human shRNA Lentiviral Particle (TL318768V) (OriGene). CRISPR reagents are well known in the art and include, for example, GA104918, GA204385, KN317964, KN317964BN, KN317964LP, KN317964RB, KN406983, and KN517964 (OriGene).

DAP10 is also known as Hematopoietic Cell Signal Transducer or HCST. DAP10 is a transmembrane signaling adaptor that contains a YxxM motif in its cytoplasmic domain. The encoded protein may form part of the immune recognition receptor complex with the C-type lectin-like receptor NKG2D. As part of this receptor complex, this protein may activate phosphatidylinositol 3-kinase dependent signaling pathways through its intracytoplasmic YxxM motif. This receptor complex may have a role in cell survival and proliferation by activation of NK and T cell responses. Alternative splicing results in two transcript variants encoding different isoforms. DAP10 is a Transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and UL16-binding proteins (ULBPs); these ligands are up-regulated by stress conditions and pathological state such as viral infection and tumor transformation. Functions as docking site for PI3-kinase PIK3R1 and GRB2. Interaction of ULBPs with KLRK1-HCST triggers calcium mobilization and activation of the PIK3R1, MAP2K/ERK, and JAK2/STAT5 signaling pathways. Both PIK3R1 and GRB2 are required for full KLRK1-HCST-mediated activation and ultimate killing of target cells. In NK cells, KLRK1-HCST signaling directly induces cytotoxicity and enhances cytokine production initiated via DAP12/TYROBP-associated receptors. In T-cells, it provides primarily costimulation for TCR-induced signals. KLRK1-HCST receptor plays a role in immune surveillance against tumors and is required for cytolysis of tumors cells; indeed, melanoma cells that do not express KLRK1 ligands escape from immune surveillance mediated by NK cells.

Anti-DAP10 antibodies are well known in the art and include, for example, DAP10 (H-2), DAP10 (C-5), DAP10 (C-1), and DAP10 (H-3) (Santa Cruz Biotechnology). Inhibitory RNA reagents are well known in the art and include, for example, HCST Human shRNA Plasmid Kit (TL319523), HCST Human siRNA Oligo Duplex (SR323260), Hcst Mouse siRNA Oligo Duplex (SR400333), Hcst Mouse shRNA Plasmid (TL519117), and HCST Human shRNA Lentiviral Particle (TL319523V) (OriGene). CRISPR reagents are well known in the art and include, for example, GA107444, KN408290, and KN507613 (OriGene); and DAP10 CRISPR/Cas9 KO Plasmid (h), DAP10 CRISPR Activation Plasmid (h), and DAP10 CRISPR Activation Plasmid (h2) (Santa Cruz Biotechnology).

DAP12 is known as Transmembrane Immune Signaling Adaptor TYROBP or TYROBP. DAP12 is a transmembrane signaling polypeptide which contains an immunoreceptor tyrosine-based activation motif (ITAM) in its cytoplasmic domain. The encoded protein may associate with the killer-cell inhibitory receptor (KIR) family of membrane glycoproteins and may act as an activating signal transduction element. This protein may bind zeta-chain (TCR) associated protein kinase 70 kDa (ZAP-70) and spleen tyrosine kinase (SYK) and play a role in signal transduction, bone modeling, brain myelination, and inflammation. Mutations within this gene have been associated with polycystic lipomembranous osteodysplasia with sclerosing leukoencephalopathy (PLOSL), also known as Nasu-Hakola disease. Its putative receptor, triggering receptor expressed on myeloid cells 2 (TREM2), also causes PLOSL. Multiple alternative transcript variants encoding distinct isoforms have been identified for this gene.

Anti-DAP12 antibodies are well known in the art and include, for example, AP05157PU-N, AP22905PU-N, AP54428PU-N, TA319099, TA321949, and TA332657 (OriGene); AFLGC-TYROBP (Invitrogen); and orb156537, orb464992, and orb536709 (Biorbyt). Inhibitory RNA reagents are well known in the art and include, for example, DAP12 (TYROBP) Human siRNA Oligo Duplex (SR304987), DAP12 (TYROBP) Human shRNA Plasmid Kit (TL316769), DAP12 (TYROBP) Human shRNA Lentiviral Particle (TL316769V), and Tyrobp Mouse shRNA Lentiviral Particle (TL517068V) (OriGene). CRISPR reagents are well known in the art and include, for example, GA105031, GA204507, KN403771, and KN518529 (OriGene); and DAP12 CRISPR/Cas9 KO Plasmid (h), DAP12 CRISPR Activation Plasmid (h), and DAP12 CRISPR Activation Plasmid (h2) (Santa Cruz Biotechnology).

Clc-5 is also known as Chloride Voltage-Gated Channel 5 or CLCN5. Clc-5 is a member of the ClC family of chloride ion channels and ion transporters. The encoded protein is primarily localized to endosomal membranes and may function to facilitate albumin uptake by the renal proximal tubule. Mutations in this gene have been found in Dent disease and renal tubular disorders complicated by nephrolithiasis.

Anti-Clc-5 antibodies are well known in the art and include, for example, 73-400, TA308868, TA328780, TA332854, and TA338390 (OriGene); AFLGC-CLCN5 (Invitrogen); and A02286 and A02286-1 (Boster Bio). Inhibitory RNA reagents are well known in the art and include, for example, Chloride Channel 5 (CLCN5) Human shRNA Plasmid Kit (TR313877), Chloride Channel 5 (CLCN5) Human siRNA Oligo Duplex (SR319728), Chloride Channel 5 (CLCN5) Human shRNA Lentiviral Particle (TL313877V), Clcn5 Mouse shRNA Lentiviral Particle (TL500372V), and Clcn5 Rat shRNA Lentiviral Particle (TL709916V) (OriGene). CRISPR reagents are well known in the art and include, for example, GA100846, GA200748, KN417750, and KN503395 (OriGene); and CLC-5 CRISPR/Cas9 KO Plasmid (h), CLC-5 CRISPR Activation Plasmid (h), and CLC-5 CRISPR Activation Plasmid (h2) (Santa Cruz Biotechnology).

MCP-1 is also known as C-C motif chemokine ligand 2 or CCL2. MCP-1 gene is one of several cytokine genes clustered on the q-arm of chromosome 17. Chemokines are a superfamily of secreted proteins involved in immunoregulatory and inflammatory processes. The superfamily is divided into four subfamilies based on the arrangement of N-terminal cysteine residues of the mature peptide. This chemokine is a member of the CC subfamily which is characterized by two adjacent cysteine residues. This cytokine displays chemotactic activity for monocytes and basophils but not for neutrophils or eosinophils. It has been implicated in the pathogenesis of diseases characterized by monocytic infiltrates, like psoriasis, rheumatoid arthritis and atherosclerosis. It binds to chemokine receptors CCR2 and CCR4. Diseases associated with CCL2 include Neural Tube Defects and Human Immunodeficiency Virus Type 1.

Anti-MCP-1 antibodies are well known in the art and include, for example, MCP-1 (5J), MCP-1 (5J) L, MCP-1 (D9), MCP-1 (D9) L, MCP-1 (5D3-F7), MCP-1 (1A7B8), MCP-1 (S8), MCP-1 (5), and MCP-1-4/eotaxin (B-2) (Santa Cruz Biotechnology); A00056, A00056-4, PB9570, and M00056 (Boster Bio); and orb181787, orb323291, orb 11028, orb13563, orb36895, orb374685, orb97456, orb240703, and orb240704 (Biorbyt). Small molecule inhibitors are well known in the art and include, for example, danazol, atorvastatin, simvastatin, chondroitin sulfate, risperidone, and bindarit. Inhibitory RNA reagents are well known in the art and include, for example, MCP1 (CCL2) Human shRNA Plasmid Kit (TL316716), MCP1 (CCL2) Human siRNA Oligo Duplex (SR304273), MCP1 (CCL2) Human shRNA Lentiviral Particle (TL316716V), Ccl2 Rat shRNA Lentiviral Particle (TL711346V), and Ccl2 Mouse shRNA Lentiviral Particle (TL501987V) (OriGene). CRISPR reagents are well known in the art and include, for example, GA104312, GA203799, KN302782, KN302782BN, KN302782LP, KN302782RB, KN402180, and KN502782 (OriGene); and MCP-1 CRISPR/Cas9 KO Plasmid (h), MCP-1 CRISPR Activation Plasmid (h), and MCP-1 CRISPR Activation Plasmid (h2) (Santa Cruz Biotechnology).

Transglutaminase 3 is also known as transglutaminase E or TGM3. Transglutaminase are enzymes that catalyze the crosslinking of proteins by epsilon-gamma glutamyl lysine isopeptide bonds. While the primary structure of transglutaminases is not conserved, they all have the same amino acid sequence at their active sites and their activity is calcium-dependent. The protein encoded by this gene has two polypeptide chains activated from a single precursor protein by proteolysis. The encoded protein is involved the later stages of cell envelope formation in the epidermis and hair follicle. Diseases associated with TGM3 include Uncombable Hair Syndrome 2 and Uncombable Hair Syndrome 1.

Anti-Transglutaminase 3 antibodies are well known in the art and include, for example,
AM10224PU-N, AM10225PU-N, AM10226PU-N, AP02094SU-N, AP02094SU-S, AP55337PU-N, AP55344PU-N, TA309514, TA332965, and TA346349 (OriGene); orb13714 and orb12789 (Biorbyt); and A04998 (Boster Bio). Small molecule inhibitors are well known in the art and include, for example, guanosine monophosphate, 5′-guanosine-diphosphate-monothiophosphate, B-2-octylglucoside, and Cystamine dihydrochloride.
Inhibitory RNA reagents are well known in the art and include, for example,
Transglutaminase 3 (TGM3) Human siRNA Oligo Duplex (SR304818), Transglutaminase 3 (TGM3) Human shRNA Plasmid Kit (TL308846), Transglutaminase 3 (TGM3) Human shRNA Lentiviral Particle (TL308846V), Tgm3 Mouse shRNA Lentiviral Particle (TL511495V), and Tgm3 Rat shRNA Lentiviral Particle (TL707481V) (OriGene). CRISPR reagents are well known in the art and include, for example, GA104860, GA204328, KN420515, and KN517497 (OriGene); and TGase3 CRISPR/Cas9 KO Plasmid (h), TGase3 CRISPR Activation Plasmid (h), TGase3 CRISPR Activation Plasmid (h2), and TGase3 CRISPR/Cas9 KO Plasmid (h2) (Santa Cruz Biotechnology).

Presenilins 1 and 2 are also known as PSEN1 and PSEN2, respectively. Alzheimer's disease (AD) patients with an inherited form of the disease carry mutations in the presenilin proteins (PSEN1; PSEN2) or in the amyloid precursor protein (APP). These disease-linked mutations result in increased production of the longer form of amyloid-beta (main component of amyloid deposits found in AD brains). Presenilins are postulated to regulate APP processing through their effects on gamma-secretase, an enzyme that cleaves APP. Also, it is thought that the presenilins are involved in the cleavage of the Notch receptor, such that they either directly regulate gamma-secretase activity or themselves are protease enzymes. Diseases associated with PSEN1 include Pick Disease Of Brain and Alzheimer Disease 3. Diseases associated with PSEN2 include Alzheimer Disease 4 and Cardiomyopathy, Dilated, 1V.

Anti-Presenilin 1 antibodies are well known in the art and include, for example, AP06290PU-N, AP07340PU-N, AP13205PU-N, SP1373P, TA306424, TA313388, TA334642, TA336586, TA347463, and TA354271 (OriGene); and Presenilin 1 (D-10) and Presenilin 1 (H-5) (Santa Cruz Biotechnology). Small molecule inhibitors are well known in the art and include, for example, avagacestat, begacestat, GSI-136, semagacestat, BMS 299897, compound W, DAPT, JLK6, and L-685458. Inhibitory RNA reagents are well known in the art and include, for example, Presenilin 1 (PSEN1) Human siRNA Oligo Duplex (SR303805), Presenilin 1 (PSEN1) Human shRNA Plasmid Kit (TL320496), Presenilin 1 (PSEN1) Human shRNA Lentiviral Particle (TL320496V), Psen1 Rat shRNA Lentiviral Particle (TL710183V), and Psen1 Mouse shRNA Lentiviral Particle (TL511989V) (OriGene). CRISPR reagents are well known in the art and include, for example, GA103839, KN216443, KN216443BN, KN216443LP, KN216443RB, KN416443, and KN514071 (OriGene); and Presenilin 1 CRISPR/Cas9 KO Plasmid (h), Presenilin 1 CRISPR Activation Plasmid (h), and Presenilin 1 CRISPR Activation Plasmid (h2) (Santa Cruz Biotechnology).

Anti-Presenilin 2 antibodies are well known in the art and include, for example, TA335237, TA335238, and TA336445 (OriGene); orb6805, orb373712, orb381989, and orb305821 (Biorbyt); and Presenilin 2 (B-7) (Santa Cruz Biotechnology). Small molecule inhibitors are well known in the art and include, for example, GSI-136, semagacestat, BMS-299897, BMS-708163, compound W, DAPT, JLK6, and L-685458. Inhibitory RNA reagents are well known in the art and include, for example, Presenilin 2 (PSEN2) Human siRNA Oligo Duplex (SR303806), Presenilin 2 (PSEN2) Human shRNA Plasmid Kit (TL310125), Presenilin 2 (PSEN2) Human shRNA Lentiviral Particle (TL310125V), Psen2 Mouse shRNA Lentiviral Particle (TL510385V), and Psen2 Rat shRNA Lentiviral Particle (TL711211V) (OriGene). CRISPR reagents are well known in the art and include, for example, GA103840, GA203422, KN202921, KN202921BN, KN202921LP, KN202921RB, KN402921, and KN514072 (OriGene); and Presenilin 2 CRISPR/Cas9 KO Plasmid (h), Presenilin 2 CRISPR Activation Plasmid (h), and Presenilin 2 CRISPR Activation Plasmid (h2) (Santa Cruz Biotechnology).

Annexin-1 is also known as Annexin A1, Phospholipase A2 Inhibitory Protein, or ANXA1. Annexin-1 is a membrane-localized protein that binds phospholipids. This protein inhibits phospholipase A2 and has anti-inflammatory activity. Loss of function or expression of this gene has been detected in multiple tumors. Diseases associated with ANXA1 include Hairy Cell Leukemia and Brain Edema.

Anti-ANXA1 antibodies are well known in the art and include, for example, AP17109PU-N, AP20568PU-N, AP22515PU-N, AP22730PU-N, AP23249PU-N, AP54783SU-N, CF500967, TA305797, TA308701, TA313314, and TA323578 (OriGene); and Annexin I (EH17a), Annexin I (ANEX 5E4/1), Annexin I (ANEX 6E4/3), Annexin I (74/3), Annexin I (39), and p-Annexin I (86.Tyr 21) (Santa Cruz Biotechnology). Small molecule inhibitors are well known in the art and include, for example, dexamethasone, amcinonide, clobetasol, methylprednisolone, Ac2-12, Ac2-26, KF 38789, PM 102, and thiolutin. Inhibitory RNA reagents are well known in the art and include, for example, Annexin A1 (ANXA1) Human shRNA Plasmid Kit (TR314775), Annexin A1 (ANXA1) Human siRNA Oligo Duplex (SR300207), Annexin A1 (ANXA1) Human shRNA Lentiviral Particle (TL314775V), Anxa1 Mouse shRNA Lentiviral Particle (TL513843V), and Anxa1 Rat shRNA Lentiviral Particle (TL709518V) (OriGene). CRISPR reagents are well known in the art and include, for example, GA100206, KN401569, and KN501330 (OriGene); and Annexin I CRISPR/Cas9 KO Plasmid (h), Annexin I CRISPR Activation Plasmid (h), Annexin I CRISPR Activation Plasmid (h2), and Annexin I CRISPR/Cas9 KO Plasmid (h2) (Santa Cruz Biotechnology).

A20 (TNFAIP3) is also known as TNF Alpha Induced Protein 3 or TNFAIP3. This gene was identified as a gene whose expression is rapidly induced by the tumor necrosis factor (TNF). The protein encoded by this gene is a zinc finger protein and ubiqitin-editing enzyme, and has been shown to inhibit NF-kappa B activation as well as TNF-mediated apoptosis. The encoded protein, which has both ubiquitin ligase and deubiquitinase activities, is involved in the cytokine-mediated immune and inflammatory responses.

Anti-A20 antibodies is well known in the art and include, for example, AP22993PU-N, AP23477PU-N, TA306764, TA306770, TA312809, TA327286, TA336834, and TA343148 (OriGene); and A20 (8E8.38), A20 (8E8.38) X, A20 (59A426), A20 (4H16), A20 (A-12), A20 (A-12) X, A20 (B-5), and A20 (B-5) X (Santa Cruz Biotechnology). Inhibitory RNA reagents are well known in the art and include, for example, TNFAIP3 Human shRNA Plasmid Kit (TR320557), TNFAIP3 Human siRNA Oligo Duplex (SR304881), TNFAIP3 Human shRNA Lentiviral Particle (TL320557V), Tnfaip3 Mouse shRNA Plasmid (TG502299), and Tnfaip3 Mouse shRNA Lentiviral Particle (TL502299V) (OriGene). CRISPR reagents are well known in the art and include, for example, GA104922, GA204388, KN221337, KN221337BN, KN221337LP, KN221337RB, KN421337, and KN517967 (OriGene); and A20 CRISPR Activation Plasmid (h), A20 CRISPR Activation Plasmid (h2), and A20 CRISPR/Cas9 KO Plasmid (h2) (Santa Cruz Biotechnology).

Galectin-1 is also known as Lectin Galactoside-Binding Soluble 1 or LGALS1. The galectins are a family of beta-galactoside-binding proteins implicated in modulating cell-cell and cell-matrix interactions. This gene product may act as an autocrine negative growth factor that regulates cell proliferation. Diseases associated with LGALS1 include Trophoblastic Neoplasm and Fucosidosis.

Anti-galectin-1 antibodies are well known in the art and include, for example, orb330979, orb352065, orb4299, orb382609, and orb388413 (Biorbyt); and galectin-1 (B-2), galectin-1 (C-8), and galectin-1 (E-2) (Santa Cruz Biotechnology). Inhibitors of galectin-1 are well known in the art and include, for example, thiodigalactoside and dihydroartemisinin. Inhibitory RNA reagents are well known in the art and include, for example, Galectin 1 (LGALS1) Human shRNA Plasmid Kit (TL311756), Galectin 1 (LGALS1) Human siRNA Oligo Duplex (SR320840), Galectin 1 (LGALS1) Human shRNA Lentiviral Particle (TL311756V), Lgals1 Mouse shRNA Lentiviral Particle (TL515051V), and Lgals1 Rat shRNA Lentiviral Particle (TL710385V) (OriGene). CRISPR reagents are well known in the art and include, for example, galectin-1 CRISPR/Cas9 KO Plasmid (h), galectin-1 CRISPR Activation Plasmid (h), and galectin-1 CRISPR Activation Plasmid (h2) (Santa Cruz Biotechnology); and GA102681, GA202442, KN204674, KN204674BN, KN204674LP, KN204674RB, KN309240, KN309240BN, KN309240LP, KN309240RB, KN404674, and KN509240 (OriGene).

Galectin-3 is also known as Lectin Galactoside-Binding Soluble 3 or LGALS3. Galectin-3 is characterized by an N-terminal proline-rich tandem repeat domain and a single C-terminal carbohydrate recognition domain. This protein can self-associate through the N-terminal domain allowing it to bind to multivalent saccharide ligands. This protein localizes to the extracellular matrix, the cytoplasm and the nucleus. This protein plays a role in numerous cellular functions including apoptosis, innate immunity, cell adhesion and T-cell regulation. Diseases associated with LGALS3 include Follicular Adenoma and Papillary Carcinoma.

Anti-galectin-3 antibodies are well known in the art and include, for example, PB9081, PB9279, M00621, M00621-1, and M00621-3 (Boster Bio); and galectin-3 (M3/38), galectin-3 (B-2), galectin-3 (B2C10), galectin-3 (B2C10), L galectin-3 (A3A12), galectin-3 (M3/38.1.2.8 HL.2), galectin-3 (H-5), and galectin-3 (F-8) (Santa Cruz Biotechnology). Inhibitors of galectin-3 are well known in the art and include, for example, lactose and 2,3,5,6-Tetrafluoro-4-Methoxy-Benzamide. Inhibitory RNA reagents are well known in the art and include, for example, Galectin 3 (LGALS3) Human siRNA Oligo Duplex (SR302676), Galectin 3 (LGALS3) Human shRNA Plasmid Kit (TR311753), Lgals3 Mouse siRNA Oligo Duplex (SR426188), Lgals3 Mouse shRNA Lentiviral Particle (TL501241V), and Lgals3 Rat shRNA Plasmid (TL711620) (OriGene). CRISPR reagents are well known in the art and include, for example, galectin-3 CRISPR/Cas9 KO Plasmid (h), galectin-3 CRISPR Activation Plasmid (h), and galectin-3 CRISPR Activation Plasmid (h2) (Santa Cruz Biotechnology); and GA102683, GA202443, KN208785, KN208785BN, and KN208785LP (OriGene).

PGC-1a is also known as Peroxisome Proliferator-Activated Receptor Gamma Coactivator 1-Alpha or PPARGC1A. PGC-1a is a transcriptional coactivator that regulates the genes involved in energy metabolism. This protein interacts with PPARgamma, which permits the interaction of this protein with multiple transcription factors. This protein can interact with, and regulate the activities of, cAMP response element binding protein (CREB) and nuclear respiratory factors (NRFs). It provides a direct link between external physiological stimuli and the regulation of mitochondrial biogenesis, and is a major factor that regulates muscle fiber type determination. This protein may be also involved in controlling blood pressure, regulating cellular cholesterol homoeostasis, and the development of obesity. Diseases associated with PPARGC1A include Amyotrophic Lateral Sclerosis 1 and Acute Porphyria.

Anti-PGC-1a antibodies are well known in the art and include, for example, PGC-1a (4A8), PGC-1a (D-5), and PGC-1a (C-4) (Santa Cruz Biotechnology); and orb317701, orb330034, orb13647, orb100963, orb124814, orb97371, and orb247039 (Biorbyt). Small molecule inhibitors are well known in the art and include, for example, balaglitazone, GW9662, rosiglitazone maleate, and ZLN005. Inhibitory RNA reagents are well known in the art and include, for example, PGC1 alpha (PPARGC1A) Human shRNA Plasmid Kit (TG310260), PGC1 alpha (PPARGC1A) Human siRNA Oligo Duplex (SR323265), PGC1 alpha (PPARGC1A) Human shRNA Lentiviral Particle (TL310260V), Ppargc1a Rat shRNA Plasmid (TG711312), and Ppargc1a Mouse shRNA Lentiviral Particle (TL509292V) (OriGene). CRISPR reagents are well known in the art and include, for example, PGC-1a CRISPR/Cas9 KO Plasmid (h), PGC-1a CRISPR Activation Plasmid (h), and PGC-1a CRISPR Activation Plasmid (h2) (Santa Cruz Biotechnology); and GA107462, GA203342, KN212244, KN212244BN, and KN212244LP (OriGene).

LDLR is also known as Low Density Lipoprotein Receptor. The low density lipoprotein receptor (LDLR) gene family consists of cell surface proteins involved in receptor-mediated endocytosis of specific ligands. Low density lipoprotein (LDL) is normally bound at the cell membrane and taken into the cell ending up in lysosomes where the protein is degraded and the cholesterol is made available for repression of microsomal enzyme 3-hydroxy-3-methylglutaryl coenzyme A (HMG CoA) reductase, the rate-limiting step in cholesterol synthesis. At the same time, a reciprocal stimulation of cholesterol ester synthesis takes place. Mutations in this gene cause the autosomal dominant disorder, familial hypercholesterolemia.

Anti-LDLR antibodies are well known in the art and include, for example, LDLR (C7), LDLR (F-7), and LDLR (B-10) (Santa Cruz Biotechnology); and BM5053, BP5014, BP5014B, CF812507, CF812508, and CF812509 (OriGene). Small molecule inhibitors are well known in the art and include, for example, atorvastatin, lovastatin, pravastatin, tocopherol, COG 133, and porfimer. Inhibitory RNA reagents are well known in the art and include, for example, LDL Receptor (LDLR) Human shRNA Plasmid Kit (TL311766), LDL Receptor (LDLR) Human siRNA Oligo Duplex (SR320836), LDL Receptor (LDLR) Human shRNA Lentiviral Particle (TL311766V), Ldlr Mouse shRNA Plasmid (TR517160), and Ldlr Rat shRNA Plasmid (TL713442) (OriGene). CRISPR reagents are well known in the art and include, for example, LDLR CRISPR Activation Plasmid (h) LDLR CRISPR Activation Plasmid (h2) LDLR CRISPR/Cas9 KO Plasmid (h2) (Santa Cruz Biotechnology); and GA102675, GA202435, KN200006, KN200006BN, and KN200006LP (OriGene).

Gprc5a is also known as G Protein-Coupled Receptor Class C Group 5 Member A, Retinoic Acid Induced 3, or RAI3. Gprc5a is a member of the type 3 G protein-coupling receptor family, characterized by the signature 7-transmembrane domain motif. The encoded protein may be involved in interaction between retinoid acid and G protein signalling pathways. Retinoic acid plays a critical role in development, cellular growth, and differentiation. This gene may play a role in embryonic development and epithelial cell differentiation. Diseases associated with GPRC5A include Lung Mucoepidermoid Carcinoma and Lung Cancer.

Anti-Gprc5a antibodies are well known in the art and include, for example, RAI3 (F-3), RAI3 (G-6), and RAI3 (A-11) (Santa Cruz Biotechnology); orb324956 and orb539707 (Biorbyt); and A05072 (Boster Bio). Small molecule inhibitors are well known in the art and include, for example, tretinoin. Inhibitory RNA reagents are well known in the art and include, for example, RAI3 (GPRC5A) Human shRNA Plasmid Kit (TL312632), RAI3 (GPRC5A) Human shRNA Lentiviral Particle (TL312632V), RAI3 (GPRC5A) Human siRNA Oligo Duplex (SR322634), Gprc5a Mouse shRNA Lentiviral Particle (TL506990V), and Gprc5a Rat shRNA Lentiviral Particle (TL703978V) (OriGene). CRISPR reagents are well known in the art and include, for example, GA106019, GA214361, KN400118, and KN507271 (OriGene); and RAI3 CRISPR/Cas9 KO Plasmid (h), RAI3 CRISPR Activation Plasmid (h), and RAI3 CRISPR Activation Plasmid (h2) (Santa Cruz Biotechnology).

BCMO1 is also known as Beta-Carotene Oxygenase 1 or BCO1. Vitamin A metabolism is important for vital processes such as vision, embryonic development, cell differentiation, and membrane and skin protection. The BCMO1 protein is a key enzyme in beta-carotene metabolism to vitamin A. It catalyzes the oxidative cleavage of beta,beta-carotene into two retinal molecules. Diseases associated with BCO1 include Hypercarotenemia And Vitamin A Deficiency, Autosomal Dominant and Hereditary Hypercarotenemia And Vitamin A Deficiency.

Anti-BCMO1 antibodies are well known in the art and include, for example, AP50362PU-N(OriGene); Anti-BCMO1 (aa 494-543) polyclonal antibody (Creative Diagnostics); AFLGC-BCO1 (Invitrogen); orb36791 (Biorbyt); and A07016 (Boster Bio). Small molecule inhibitors are well known in the art and include, for example, retinol acetate, 15, 15′-dihydroxy-beta-carotene, 9-cis-Retinal, and beta-carotene-15, 15′-epoxide.

Inhibitory RNA reagents are well known in the art and include, for example, BCMO1 (BCO1) Human shRNA Plasmid Kit (TL306416), BCMO1 (BCO1) Human siRNA Oligo Duplex (SR309991), BCMO1 (BCO1) Human shRNA Lentiviral Particle (TL306416V), Bco1 Mouse shRNA Lentiviral Particle (TL503442V), and Bco1 Rat shRNA Lentiviral Particle (TL712042V) (OriGene). CRISPR reagents are well known in the art and include, for example, GA110018, GA206414, KN423790, and KN502118 (OriGene).

PAP/HIP is also known as PAP, HIP, Regenerating Family Member 3 Alpha, or REG3A. PAP/HIP is a pancreatic secretory protein that may be involved in cell proliferation or differentiation. It has similarity to the C-type lectin superfamily. The enhanced expression of this gene is observed during pancreatic inflammation and liver carcinogenesis. The mature protein also functions as an antimicrobial protein with antibacterial activity. Diseases associated with REG3A include Pancreatitis and Acute Pancreatitis.

Anti-PAP/HIP antibodies are well known in the art and include, for example, AP53629PU-N, TA332505, TA339881, TA351397, and TA351398 (OriGene); Reg3a (AbCam); AFLGC-Reg3a (Invitrogen); and A05686 and A05686-1 (Boster Bio). Inhibitory RNA reagents are well known in the art and include, for example, REG3A Human siRNA Oligo Duplex (SR303353), REG3A Human shRNA Plasmid Kit (TL302040), REG3A Human shRNA Lentiviral Particle (TL302040V), Reg3a Mouse shRNA Lentiviral Particle (TL512300V), and Reg3a Rat shRNA Lentiviral Particle (TL709137V) (OriGene). CRISPR reagents are well known in the art and include, for example, GA103384, KN407791, and KN514650 (OriGene); and Reg IIIa CRISPR/Cas9 KO Plasmid (h), Reg IIIa CRISPR Activation Plasmid (h), and Reg IIIa CRISPR Activation Plasmid (h2) (Santa Cruz Biotechnology).

ApoE is also known as Apolipoprotein E or Alzheimer Disease 2. ApoE is a major apoprotein of the chylomicron. It binds to a specific liver and peripheral cell receptor, and is essential for the normal catabolism of triglyceride-rich lipoprotein constituents. This gene maps to chromosome 19 in a cluster with the related apolipoprotein C1 and C2 genes. Mutations in this gene result in familial dysbetalipoproteinemia, or type III hyperlipoproteinemia (HLP III), in which increased plasma cholesterol and triglycerides are the consequence of impaired clearance of chylomicron and VLDL remnants. Diseases associated with APOE include Lipoprotein Glomerulopathy and Hyperlipoproteinemia, Type Iii.

Anti-ApoE antibodies are well known in the art and include, for example, Human Apolipoprotein E/ApoE Antibody (AF4144) (R&D Systems); apoE (A1.4), apoE (WU E-4), apoE (F-9), and apoE (E-8) (Santa Cruz Biotechnology); and A00015, A00015-1, A00015-3, A00015-4, PB9327, M00015, and PB9986 (Boster Bio). Small molecule and peptide inhibitors are well known in the art and include, for example, atorvastatin, donepezil, fluvastatin, galantamine, and COG133. Inhibitory RNA reagents are well known in the art and include, for example, Apolipoprotein E (APOE) Human shRNA Plasmid Kit (TR314721), Apolipoprotein E (APOE) Human siRNA Oligo Duplex (SR300248), Apoe Mouse shRNA Plasmid (TG500118), Apoe Mouse siRNA Oligo Duplex (SR409575), and Apoe Rat shRNA Lentiviral Particle (TL712892V) (OriGene). CRISPR reagents are well known in the art and include, for example, GA100247, GA200245, KN200395, KN200395BN, KN200395LP, KN200395RB, KN400395, and KN501421 (OriGene); and apoE CRISPR/Cas9 KO Plasmid (h), apoE CRISPR Activation Plasmid (h), apoE CRISPR Activation Plasmid (h2), and apoE CRISPR/Cas9 KO Plasmid (h2) (Santa Cruz Biotechnology).

IL-6 is also known as interleukin 6. IL-6 is a cytokine that functions in inflammation and the maturation of B cells. In addition, the encoded protein has been shown to be an endogenous pyrogen capable of inducing fever in people with autoimmune diseases or infections. The protein is primarily produced at sites of acute and chronic inflammation, where it is secreted into the serum and induces a transcriptional inflammatory response through interleukin 6 receptor, alpha. The functioning of this gene is implicated in a wide variety of inflammation-associated disease states, including susceptibility to diabetes mellitus and systemic juvenile rheumatoid arthritis.

Anti-IL-6 antibodies are well known in the art and include, for example, Human/Primate IL-6 Antibody (MAB206) and Porcine IL-6 Antibody (MAB6863) (R&D Systems); AFLGC-116 (Invitrogen); orb315792, orb303674, orb10911, orb10913, orb9058, and orb345102 (Biorbyt); and PA1352, A00102-1, A00102-2, and A00102-3 (Boster Bio). Inhibitory RNA reagents are well known in the art and include, for example, IL6 Human shRNA Plasmid Kit (TL312162), IL6 Human siRNA Oligo Duplex (SR302379), IL6 Human shRNA Lentiviral Particle (TL312162V), 116 Mouse shRNA Plasmid (TG501087), and 116 Rat shRNA Plasmid (TG709233) (OriGene). CRISPR reagents are well known in the art and include, for example, IL-6 CRISPR/Cas9 KO Plasmid (h), IL-6 CRISPR Activation Plasmid (h), and IL-6 CRISPR Activation Plasmid (h2) (Santa Cruz Biotechnology); and GA102386, GA202149, KN202078, KN202078BN, and KN202078LP (OriGene).

IL-8 is also known as C-X-C motif chemokine ligand 8, interleukin 8, or CXCL8. IL-8 is a member of the CXC chemokine family and is a major mediator of the inflammatory response. IL-8 is secreted by mononuclear macrophages, neutrophils, eosinophils, T lymphocytes, epithelial cells, and fibroblasts. It functions as a chemotactic factor by guiding the neutrophils to the site of infection. Bacterial and viral products rapidly induce IL-8 expression. IL-8 also participates with other cytokines in the proinflammatory signaling cascade and plays a role in systemic inflammatory response syndrome (SIRS). This gene is believed to play a role in the pathogenesis of the lower respiratory tract infection bronchiolitis, a common respiratory tract disease caused by the respiratory syncytial virus (RSV). The overproduction of this proinflammatory protein is thought to cause the lung inflammation associated with cystic fibrosis. This proinflammatory protein is also suspected of playing a role in coronary artery disease and endothelial dysfunction. This protein is also secreted by tumor cells and promotes tumor migration, invasion, angiogenesis and metastasis. This chemokine is also a potent angiogenic factor. The binding of IL-8 to one of its receptors (IL-8RB/CXCR2) increases the permeability of blood vessels and increasing levels of IL-8 are positively correlated with increased severity of multiple disease outcomes (e.g., sepsis). This gene and other members of the CXC chemokine gene family form a gene cluster in a region of chromosome 4q.

Anti-IL-8 antibodies are well known in the art and include, for example, Human IL-8/CXCL8 Antibody (MAB208) (R&D Systems); IL-8 Antibody (511501) and IL-8 Antibody (511406) (BioLegend); AM00964AF-N and AM05672FC-N(OriGene); and orb322984 and orb135736 (Biorbyt). Inhibitory RNA reagents are well known in the art and include, for example, IL8 (CXCL8) Human shRNA Plasmid Kit (TL319476), IL8 (CXCL8) Human siRNA Oligo Duplex (SR302384), and IL8 (CXCL8) Human shRNA Lentiviral Particle (TL319476V) (OriGene). CRISPR reagents are well known in the art and include, for example, GA102391, KN202075, KN202075BN, KN202075LP, KN202075RB, and KN402075 (OriGene).

GSK3 is also known as glycogen synthase kinase 3. GSK3 is a serine/threonine protein kinase that mediates the addition of phosphate molecules onto serine and threonine amino acid residues. In mammals, including humans, GSK3 exists in two isoforms encoded by two paralogous genes GSK3a and GSK30. GSK-3 has been the subject of much research since it has been implicated in a number of diseases, including type 2 diabetes, Alzheimer's disease, inflammation, cancer, and bipolar disorder. GSK3 controls several regulatory proteins including glycogen synthase, and transcription factors, such as JUN. It also plays a role in the WNT and PI3K signaling pathways, as well as regulates the production of beta-amyloid peptides associated with Alzheimer's disease.

Anti-GSK3α and anti-GSK3β antibodies are well known in the art and include, for example, GSK-3α (H-12), GSK-3α/β (0011-A), GSK-3α/β (1H8), GSK-3α (9D5G1), GSK-3α (10C1), p-GSK-3α/β (6D3), p-GSK-3α (9B8), GSK-3α (B-8), p-GSK-3α (E-2), GSK-3α/β (0011-A), and GSK-3β (1F7) (Santa Cruz Biotechnology). Small molecule inhibitors are well known in the art and include, for example, fostamatinib, citalopram, fluoxetine, indirubin-3′-oxime, SB 216763, 1-azakenpaullone, AR-A014418, AZD1080, AZD2858, CHIR-124, and tideglusib. Inhibitory RNA reagents are well known in the art and include, for example, GSK3 alpha (GSK3A) Human shRNA Plasmid Kit (TR320375), GSK3 alpha (GSK3A) Human siRNA Oligo Duplex (SR301978), GSK3 alpha (GSK3A) Human shRNA Lentiviral Particle (TL320375V), GSK3 beta (GSK3B) Human siRNA Oligo Duplex (SR301979), GSK3 beta (GSK3B) Human shRNA Plasmid Kit (TG320376), and GSK3 beta (GSK3B) Human shRNA Lentiviral Particle (TL320376V) (OriGene). CRISPR reagents are well known in the art and include, for example, GA101982, GA217891, KN408698, KN507380, GA101983, and GA206046 (OriGene); and GSK-3α CRISPR/Cas9 KO Plasmid (h), GSK-3α CRISPR Activation Plasmid (h), GSK-3α CRISPR Activation Plasmid (h2), GSK-3β CRISPR Activation Plasmid (h), GSK-3β CRISPR Activation Plasmid (h2), and GSK-3β CRISPR/Cas9 KO Plasmid (h2) (Santa Cruz Biotechnology).

ADAM17 is also known as ADAM Metallopeptidase Domain 17 or TACE. ADAM17 is a member of the ADAM (a disintegrin and metalloprotease domain) family. Members of this family are membrane-anchored proteins structurally related to snake venom disintegrins, and have been implicated in a variety of biologic processes involving cell-cell and cell-matrix interactions, including fertilization, muscle development, and neurogenesis. The encoded preproprotein is proteolytically processed to generate the mature protease. The encoded protease functions in the ectodomain shedding of tumor necrosis factor-alpha, in which soluble tumor necrosis factor-alpha is released from the membrane-bound precursor. This protease also functions in the processing of numerous other substrates, including cell adhesion proteins, cytokine and growth factor receptors and epidermal growth factor (EGF) receptor ligands. ADAM17 also plays a prominent role in the activation of the Notch signaling pathway. Elevated expression of this gene has been observed in specific cell types derived from psoriasis, rheumatoid arthritis, multiple sclerosis and Crohn's disease patients.

Anti-ADAM17 antibodies are well known in the art and include, for example, AM20948PU-N and AP05095PU-N(OriGene); Human TACE/ADAM17 Ectodomain Antibody (MAB9301) (R&D Systems); orb18689 and orb394667 (Biorbyt); and PA1844 and A00604 (Boster Bio). Small molecule inhibitors are well known in the art and include, for example, (1S,3R,6S)-4-oxo-6-{4-[(2-phenylquinolin-4-yl)methoxy]phenyl}-5-azaspiro[2.4]heptane-1-carboxylic acid, (2R)—N-HYDROXY-2-[(3S)-3-METHYL-3-{4-[(2-METHYLQUINOLIN-4-YL)METHOXY]PHENYL}-2-OXOPYRROLIDIN-1-YL]PROPANAMIDE, (3S)-1-{[4-(but-2-yn-1-yloxy)phenyl]sulfonyl}pyrrolidine-3-thiol, (3S)-4-{[4-(BUT-2-YNYLOXY)PHENYL]SULFONYL}-N-HYDROXY-2,2-DIMETHYLTHIOMORPHOLINE-3-CARBOXAMIDE, 3-{[4-(but-2-yn-1-yloxy)phenyl]sulfonyl}propane-1-thiol; and TAPI-1. Inhibitory RNA reagents are well known in the art and include, for example, ADAM17 Human siRNA Oligo Duplex (SR321951), ADAM17 Human shRNA Plasmid Kit (TL316745), ADAM17 Human shRNA Lentiviral Particle (TL316745V), Adam17 Mouse shRNA Plasmid (TL513792), and Adam17 Rat shRNA Lentiviral Particle (TL710418V) (OriGene). CRISPR reagents are well known in the art and include, for example, GA104727, GA200063, and KN500818 (OriGene); and TACE CRISPR/Cas9 KO Plasmid (h), TACE CRISPR Activation Plasmid (h), TACE CRISPR Activation Plasmid (h2), and TACE CRISPR/Cas9 KO Plasmid (h2) (Santa Cruz Biotechnology).

NKG2A is also known as Killer Cell Lectin Like Receptor C1 or KLRC1. NKG2A is belongs to the killer cell lectin-like receptor family, which is a group of transmembrane proteins preferentially expressed in NK cells. This family of proteins is characterized by the type II membrane orientation and the presence of a C-type lectin domain. This protein forms a complex with another family member, KLRD1/CD94, and has been implicated in the recognition of the MHC class I HLA-E molecules in NK cells. The genes of NKG2 family members form a killer cell lectin-like receptor gene cluster on chromosome 12.

Anti-NKG2A antibodies are well known in the art and include, for example, AP18087PU-N, AP52396PU-N, TA308317, TA314139, TA343145, and TA349416 (OriGene); and orb353006 and orb228500 (Biorbyt). Inhibitory RNA reagents are well known in the art and include, for example, NKG2A (KLRC1) Human siRNA Oligo Duplex (SR302583), NKG2A (KLRC1) Human shRNA Plasmid Kit (TL311863), and NKG2A (KLRC1) Human shRNA Lentiviral Particle (TL311863V) (OriGene). CRISPR reagents are well known in the art and include, for example, GA102590 and GA202353 (OriGene); and NKG2-A CRISPR/Cas9 KO Plasmid (h), NKG2-A CRISPR Activation Plasmid (h), and NKG2-A CRISPR Activation Plasmid (h2) (Santa Cruz Biotechnology).

CD3 zeta is also known as CD247 or T-Cell Surface Glycoprotein CD3 Zeta Chain. CD3 zeta is T-cell receptor zeta, which together with T-cell receptor alpha/beta and gamma/delta heterodimers, and with CD3-gamma, -delta and -epsilon, forms the T-cell receptor-CD3 complex. The zeta chain plays an important role in coupling antigen recognition to several intracellular signal-transduction pathways. Low expression of the antigen results in impaired immune response. Two alternatively spliced transcript variants encoding distinct isoforms have been found for this gene.

Anti-CD3 zeta antibodies are well known in the art and include, for example, CD247 (TCRzeta, CD3zeta) Antibody (644102), CD247 (TCRzeta, CD3zeta) Antibody (644104), CD247 (TCRzeta, CD3zeta) Antibody (644101), CD247 (TCRzeta, CD3zeta) Antibody (644103), CD247 (TCRzeta, CD3zeta) Antibody (644105), and CD247 (TCRzeta, CD3zeta) Antibody (644106) (BioLegend). Inhibitors of CD3 zeta are known in the art and include muromonab and MT-103. Inhibitory RNA reagents are well known in the art and include, for example, CD247 Human shRNA Plasmid Kit (TL305517), CD247 Human siRNA Oligo Duplex (SR300648), and CD247 Human shRNA Lentiviral Particle (TL305517V) (OriGene). CRISPR reagents are well known in the art and include, for example, GA100646, GA200624, KN406562, and KN502889 (OriGene); and CD3-ζ CRISPR/Cas9 KO Plasmid (h), CD3-ζ CRISPR Activation Plasmid (h), and CD3-ζ CRISPR Activation Plasmid (h2) (Santa Cruz Biotechnology).

MCT4 is also known as Solute Carrier Family 16 Member 4 or SLC16A4. MCT4 is a proton-linked monocarboxylate transporter. MCT4 catalyzes the rapid transport across the plasma membrane of many monocarboxylates such as lactate, pyruvate, branched-chain oxo acids derived from leucine, valine and isoleucine, and the ketone bodies acetoacetate, beta-hydroxybutyrate and acetate. Diseases associated with SLC16A4 include Lacrimal Gland Carcinoma and Breast Papillary Carcinoma.

Anti-MCT4 antibodies are well known in the art and include, for example, TA322711 (OriGene); SLC16A4 (Abcam); and orb382041, orb137961, and orb536598 (Biorbyt). Inhibitory RNA reagents are well known in the art and include, for example, SLC16A4 Human siRNA Oligo Duplex (SR306029), SLC16A4 Human shRNA Plasmid Kit (TL309403), and SLC16A4 Human shRNA Lentiviral Particle (TL309403V) (OriGene). CRISPR reagents are well known in the art and include, for example, GA106075, GA214103, KN420156, and KN515890 (OriGene); and MCT5 CRISPR/Cas9 KO Plasmid (h), MCT5 CRISPR Activation Plasmid (h), and MCT5 CRISPR Activation Plasmid (h2) (Santa Cruz Biotechnology).

c-CBL is also known as Cbl Proto-Oncogene. c-CBL is a proto-oncogene that encodes a RING finger E3 ubiquitin ligase. The encoded protein is one of the enzymes required for targeting substrates for degradation by the proteasome. This protein mediates the transfer of ubiquitin from ubiquitin conjugating enzymes (E2) to specific substrates. This protein also contains an N-terminal phosphotyrosine binding domain that allows it to interact with numerous tyrosine-phosphorylated substrates and target them for proteasome degradation. As such it functions as a negative regulator of many signal transduction pathways. This gene has been found to be mutated or translocated in many cancers including acute myeloid leukaemia, and expansion of CGG repeats in the 5′ UTR has been associated with Jacobsen syndrome. Mutations in this gene are also the cause of Noonan syndrome-like disorder.

Anti-c-CBL antibodies are well known in the art and include, for example, Human/Mouse CBL Antibody (AF5998) (R&D Systems); AM06661SU-N and AP06426PU-N (OriGene); orb304710 and orb15017 (Biorbyt); and A00152 and A00152-1 (Boster Bio). Inhibitory RNA reagents are well known in the art and include, for example, CBL Human siRNA Oligo Duplex (SR300613), CBL Human shRNA Plasmid Kit (TR314179), and CBL Human shRNA Lentiviral Particle (TL314179V) (OriGene). CRISPR reagents are well known in the art and include, for example, GA100611, GA200561, KN414069, and KN502595 (OriGene); and Cbl CRISPR/Cas9 KO Plasmid (h), Cbl CRISPR Activation Plasmid (h), and Cbl CRISPR Activation Plasmid (h2) (Santa Cruz Biotechnology).

EZH2 is also known as Enhancer Of Zeste 2 Polycomb Repressive Complex 2 Subunit. EZH2 is a member of the Polycomb-group (PcG) family. PcG family members form multimeric protein complexes, which are involved in maintaining the transcriptional repressive state of genes over successive cell generations. This protein associates with the embryonic ectoderm development protein, the VAV1 oncoprotein, and the X-linked nuclear protein. This protein may play a role in the hematopoietic and central nervous systems. Diseases associated with EZH2 include Weaver Syndrome and Mantle Cell Lymphoma.

Anti-EZH2 antibodies are well known in the art and include, for example, CF802907 and CF803011 (OriGene); Human/Mouse EZH2 Antibody (AF4767) (R&D Systems); orb329985 and orb411831 (Biorbyt); and ENX-1 (D-8), ENX-1 (D-8) X, ENX-1 (C-1), and ENX-1 (C-1) X (Santa Cruz Biotechnology). Small molecule inhibitors are well known in the art and include, for example, tazemetostat, s-adenosylhomocysteine, CPI-1205, Ell, s-adenosylmethionine, 3-Deazaneplanocin (DZNep), CPI-169, and EPZ005687. Inhibitory RNA reagents are well known in the art and include, for example, EZH2 Human shRNA Plasmid Kit (TG304713), EZH2 Human siRNA Oligo Duplex (SR320099), and EZH2 Human shRNA Lentiviral Particle (TL304713V) (OriGene). CRISPR reagents are well known in the art and include, for example, GA101494, GA201330, KN202054, KN202054BN, KN202054LP, KN202054RB, KN402054, and KN505462 (OriGene); and ENX-1 CRISPR Activation Plasmid (h), ENX-1 CRISPR Activation Plasmid (h2), and ENX-1 CRISPR/Cas9 KO Plasmid (h2) (Santa Cruz Biotechnology).

SUP-1 is also known as Protein Tyrosine Phosphatase Non-Receptor Type 6 or PTPN6. SHP-1 is a member of the protein tyrosine phosphatase (PTP) family. PTPs are known to be signaling molecules that regulate a variety of cellular processes including cell growth, differentiation, mitotic cycle, and oncogenic transformation. N-terminal part of this PTP contains two tandem Src homolog (SH2) domains, which act as protein phosphotyrosine binding domains, and mediate the interaction of this PTP with its substrates. This PTP is expressed primarily in hematopoietic cells, and functions as an important regulator of multiple signaling pathways in hematopoietic cells. This PTP has been shown to interact with, and dephosphorylate a wide spectrum of phospho-proteins involved in hematopoietic cell signaling. Diseases associated with PTPN6 include Cd45 Deficiency and Polycythemia.

Anti-SHP-1 antibodies are well known in the art and include, for example, Human/Mouse/Rat SUP-1 Antibody (AF1878) (R&D Systems); AM09319PU-N and AM09320PU-N(OriGene); orb338828 and orb14690 (Biorbyt); and A00938 and A00938Y536 (Boster Bio). Small molecule inhibitors are well known in the art and include, for example, tiludronate, BVT 948, NSC 87877, sodium orthovanadate, TCS 401, and etidronate. Inhibitory RNA reagents are well known in the art and include, for example, SUP1 (PTPN6) Human shRNA Plasmid Kit (TG310057), SUP1 (PTPN6) Human siRNA Oligo Duplex (SR321517), and SHP1 (PTPN6) Human shRNA Lentiviral Particle (TL310057V) (OriGene). CRISPR reagents are well known in the art and include, for example, GA103923, GA201859, KN400728, and KN514220 (OriGene); and SH-PTP1 CRISPR Activation Plasmid (h), SH-PTP1 CRISPR Activation Plasmid (h2), and SH-PTP1 CRISPR/Cas9 KO Plasmid (h2) (Santa Cruz Biotechnology).

Cbl-b is also known as Cbl Proto-Oncogene B or CBLB. Cbl-b is an E3 ubiquitin-protein ligase which promotes proteosome-mediated protein degradation by transferring ubiquitin from an E2 ubiquitin-conjugating enzyme to a substrate. The encoded protein is involved in the regulation of immune response by limiting T-cell receptor, B-cell receptor, and high affinity immunoglobulin epsilon receptor activation. Studies in mouse suggest that this gene is involved in antifungal host defense and that its inhibition leads to increased fungal killing. Manipulation of this gene may be beneficial in implementing immunotherapies for a variety of conditions, including cancer, autoimmune diseases, allergies, and infections. Diseases associated with CBLB include Lymphoma and Juvenile Myelomonocytic Leukemia.

Anti-Cbl-b antibodies are well known in the art and include, for example, TA327228 (OriGene); orb101851 and orb135181 (Biorbyt); and A00735 (Boster Bio). Inhibitory RNA reagents are well known in the art and include, for example, CBLB Human siRNA Oligo Duplex (SR300614), CBLB Human shRNA Plasmid Kit (TL316477), and CBLB Human shRNA Lentiviral Particle (TL316477V) (OriGene). CRISPR reagents are well known in the art and include, for example, GA100612, GA212882, KN406047, and KN502596 (OriGene); and Cbl-b CRISPR/Cas9 KO Plasmid (h), Cbl-b CRISPR Activation Plasmid (h), and Cbl-b CRISPR Activation Plasmid (h2) (Santa Cruz Technology).

Representative human cDNA and protein sequences of the biomarkers of the present disclosure are well-known in the art and are publicly available from the National Center for Biotechnology Information (NCBI).

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequences, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### Antibody

Unless otherwise specified here within, the terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies or intracellular immunoglobulin molecules are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, biomarker peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be humanized, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, have been grafted onto human framework sequences.

### Cancer

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

### Immune Cells in General

Immune cells refer to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

Macrophages (and their precursors, monocytes) are the ‘big eaters’ of the immune system. These cells reside in every tissue of the body, albeit in different guises, such as microglia, Kupffer cells and osteoclasts, where they engulf apoptotic cells and pathogens and produce immune effector molecules. Upon tissue damage or infection, monocytes are rapidly recruited to the tissue, where they differentiate into tissue macrophages. Macrophages are remarkably plastic and can change their functional phenotype depending on the environmental cues they receive. Through their ability to clear pathogens and instruct other immune cells, these cells have a central role in protecting the host but also contribute to the pathogenesis of inflammatory and degenerative diseases. Macrophages that encourage inflammation are called M1 macrophages, whereas those that decrease inflammation and encourage tissue repair are called M2 macrophages. M1 macrophages are activated by LPS and IFN-gamma, and secrete high levels of IL-12 and low levels of IL-10. M2 is the phenotype of resident tissue macrophages, and can be further elevated by IL-4. M2 macrophages produce high levels of IL-10, TGFβ and low levels of IL-12. Tumor-associated macrophages are mainly of the M2 phenotype, and seem to actively promote tumor growth.

Myeloid derived suppressor cells (MDSCs) are an intrinsic part of the myeloid cell lineage and are a heterogeneous population comprised of myeloid cell progenitors and precursors of granulocytes, macrophages and dendritic cells. MDSCs are defined by their myeloid origin, immature state and ability to potently suppress T cell responses. They regulate immune responses and tissue repair in healthy individuals and the population rapidly expands during inflammation, infection and cancer. MDSC are one of the major components of the tumor microenvironment. The main feature of these cells is their potent immune suppressive activity. MDSC are generated in the bone marrow and, in tumor-bearing hosts, migrate to peripheral lymphoid organs and the tumor to contribute to the formation of the tumor microenvironment. This process is controlled by a set of defined chemokines, many of which are upregulated in cancer. Hypoxia appears to have a critical role in the regulation of MDSC differentiation and function in tumors. Therapeutic strategies are now being developed to target MDSCs to promote antitumour immune responses or to inhibit immune responses in the setting of autoimmune disease or transplant rejection.

Dendritic cells (DCs) are professional antigen-presenting cells located in the skin, mucosa and lymphoid tissues. Their main function is to process antigens and present them to T cells to promote immunity to foreign antigens and tolerance to self antigens. They also secrete cytokines to regulate immune responses.

Conventional T cells, also known as Tconv or Teffs, have effector functions (e.g., cytokine secretion, cytotoxic activity, anti-self-recognition, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+ T lymphocytes. “Naïve Tcons” are CD4+ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

### NK Cells

Natural killer cells or NK cells are a type of cytotoxic lymphocyte critical to the innate immune system. The role NK cells play is analogous to that of cytotoxic T cells in the vertebrate adaptive immune response. NK cells provide rapid responses to viral-infected cells, acting at around 3 days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. This role is especially important because harmful cells that are missing MHC I markers cannot be detected and destroyed by other immune cells, such as T lymphocyte cells.

NK cells (belonging to the group of innate lymphoid cells) are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter into the circulation. NK cells differ from natural killer T cells (NKTs) phenotypically, by origin and by respective effector functions; often, NKT cell activity promotes NK cell activity by secreting IFNγ. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. The NKp46 cell surface marker constitutes, at the moment, another NK cell marker of preference being expressed in both humans, several strains of mice (including BALB/c mice) and in three common monkey species.

NK cells are negatively regulated by major histocompatibility complex (MHC) class I-specific inhibitory receptors (Karre et al., 1986; Ohlen et al, 1989). These specific receptors bind to polymorphic determinants of MHC class I molecules or HLA present on other cells and inhibit NK cell lysis. In humans, certain members of a family of receptors termed killer Ig-like receptors (KIRs) recognize groups of HLA class I alleles.

KIRs are a large family of receptors present on certain subsets of lymphocytes, including NK cells. The nomenclature for KIRs is based upon the number of extracellular domains (KIR2D or KIR3D) and whether the cytoplasmic tail is either long (KIR2DL or KIR3DL) or short (KIR2DS or KIR3DS). Within humans, the presence or absence of a given KIR is variable from one NK cell to another within the NK population present in a single individual. Within the human population there is also a relatively high level of polymorphism of the KIR molecules, with certain KIR molecules being present in some, but not all individuals. Certain KIR gene products cause stimulation of lymphocyte activity when bound to an appropriate ligand. The confirmed stimulatory KIRs all have a short cytoplasmic tail with a charged transmembrane residue that associates with an adapter molecule having an immunostimulatory motif (ITAM). Other KIR gene products are inhibitory in nature.

### Dendritic Cells

Dendritic cells (DCs) are antigen-presenting cells (also known as accessory cells) of the mammalian immune system. Their main function is to process antigen material and present it on the cell surface to the T cells of the immune system. They act as messengers between the innate and the adaptive immune systems.

Dendritic cells are present in those tissues that are in contact with the external environment, such as the skin (where there is a specialized dendritic cell type called the Langerhans cell) and the inner lining of the nose, lungs, stomach and intestines. They can also be found in an immature state in the blood. Once activated, they migrate to the lymph nodes where they interact with T cells and B cells to initiate and shape the adaptive immune response. At certain development stages they grow branched projections, the dendrites that give the cell its name. While similar in appearance, these are structures distinct from the dendrites of neurons. Immature dendritic cells are also called veiled cells, as they possess large cytoplasmic “veils” rather than dendrites.

### Osteoclasts

Osteoclasts are a type of bone cell, derived from hematopoietic stem cells. Their function, resorbing bone tissue, is critical for the maintenance, repair, and remodeling of bones. Bone homeostasis is achieved when there is a balance between osteoblast bone formation and osteoclast bone resorption. Osteoclasts mature through stimulation from osteoblasts expressing RANKL, and their interaction, mediated by firm adhesion via ICAM-1. Osteoclasts also express many ligands for receptors present on activated NK cells. They reported that osteoclasts express ULBP-1, ULBP-2/5/6 and ULBP-3, but little or no MIC-A, MIC-B, or MHC class I-like ligands for NKG2D, the activating receptor of NK cells.

Osteoclasts (OCs), in comparison to dendritic cells (DCs) and monocytes, are significant activators of NK cell expansion and function (Tseng et al. (2015) Oncotarget 6(24):20002-25). Additionally, osteoclasts secrete significant amounts of IL-12, IL-15, IFN-γ and IL-18, which are known to activate NK cells; osteoclasts also express important NK-activating ligands. The instant disclosure provides a disclosure on how to expand highly functional, super-charged, osteoclast-expanded NK cells to levels that are significantly higher than those established by other methodologies. Several in vitro NK expansion techniques have been developed to establish a higher therapeutic cell dosage, while boosting activity and in vivo proliferative potential of NK cells. Some of these techniques include the stimulation of peripheral blood mononuclear cells (PBMCs), PBMC-purified populations of NK cells, or the use of human cord blood, sometimes in combination with various feeder cells such as K562 cells expressing membrane-bound IL-15 and 41BB ligand (K562-mb15-41BBL), EBV-TM-LCL, Wilms tumor or irradiated PBMCs. These studies have generated clinically relevant NK cell numbers that have good function.

### Probiotic Bacteria

In some embodiments, the instant invention is drawn to a composition comprising at least one probiotic bacterial strain, capable of regulating NK cell function. Such probiotic bacteria induce significant split anergy in activated NK cells, leading to a significant induction of IFN-γ and TNF-α. In addition, such probiotic bacteria induce significant expansion of NK cells.

Many commercial probiotics are available, having various effects of reducing gastrointestinal discomfort or strengthening of the immune system. Preferred probiotic bacteria species for use in the compositions and methods described herein include those commercially available strains of probiotic bacteria (such as sAJ2 bacteria), especially those from the Streptococcus (e.g., S. thermophiles), Bifidobacterium (e.g., B. longum, B. breve, B. infantis, B. breve, B. infantis), and Lactobacillus genera (e.g., L. acidophilus, L. helveticus, L. bulgaricus, L. rhamnosus, L. plantarum, and L. paracasei). The instant disclosure comprising methods of administering at least one probiotic bacterial strain, preferably a combination of two or more different bacterial strains, to a subject, preferably a mammal (e.g., a human). Such administration may be systemically or locally (e.g., directly to intestines) performed. A preferably administration route is oral administration. Other routes (e.g., rectal) may be also used. For administration, either the bacteria (e.g., in a wet, sonicated, grounded, or dried form or formula), the bacterial culture medium containing the bacteria, or the bacterial culture medium supernatant (not containing the bacteria), may be administered.

### Cancer Vaccine

The present invention provides a cancer vaccine comprising cancer cells, wherein the cancer cells have a decreased copy number, amount, and/or activity of at least one biomarker that is important for differentiation of the cancer cells. The present invention also provides a cancer vaccine comprising cancer cells, wherein the cancer cells have a decreased copy number, amount, and/or activity of at least one biomarker selected from: Cox2, Rag2, NFkB, STAT3, CD133, NEMO, TNF-a, DAP10, DAP12, Clc-5, MCP-1, Transglutaminase 3, Presenilin 1, Presenilin 2, Annexin-1, A20 (TNFAIP3), Galectin-1, Galectin-3, PGC-1a, LDLR, Abca1, Gprc5a, BCMO1, PAP/HIP, ApoE, IL-6, IL-8, GSK3, ADAM17, NKG2A, CD3 zeta, MCT4, c-CBL, EZH2, SUP-1, and Cbl-b. In some embodiments, the copy number, amount, and/or activity of at least one biomarker is decreased in the cancer cells by contacting the cancer cells with a small molecule inhibitor, CRISPR guide RNA (gRNA), RNA interfering agent, antisense oligonucleotide, peptide or peptidomimetic inhibitor, aptamer, antibody, and/or intrabody.

a. Cancer Cell Isolation and Purification

In some embodiments, the cancer cells are derived from a subject. Isolation and purification of tumor cell from various tumor tissues such as surgical tumor tissues, ascites or carcinous hydrothorax is a common process to obtain the purified tumor cells. Cancer cells may be purified from fresh biopsy samples from cancer patients or animal tumor models. The biopsy samples often contain a heterogeneous population of cells that include normal tissue, blood, and cancer cells. Preferably, a purified cancer cell composition can have greater than 10%, 20% 30%, 40%, 50%, 60%, 70%, 75%, 80%, 85%, 90%, 95%, 99%, or more, or any range in between or any value in between, total viable cancer cells. To purify cancer cells from the heterogeneous population, a number of methods can be used.

In some embodiments, laser microdissection is used to isolate cancer cells. Cancer cells of interest can be carefully dissected from thin tissue slices prepared for microscopy. In this method, the tissue section is coated with a thin plastic film and an area containing the selected cells is irradiated with a focused infrared laser beam pulse. This melts a small circle in the plastic film, causing cell binding underneath. Those captured cells are removed for additional analysis. This technique is good for separating and analyzing cells from different parts of a tumor, which allows for a comparison of their similar and distinct properties. It was used recently to analyze pituitary cells from dissociated tissues and from cultured populations of heterogeneous pituitary, thyroid, and carcinoid tumor cells, as well as analyzing single cells found in various sarcomas.

In other embodiments, fluorescence activated cell sorting (FACS), also referred to as flow cytometry, is used to sort and analyze the different cell populations. Cells having a cellular marker or other specific marker of interest are tagged with an antibody, or typically a mixture of antibodies, that bind the cellular markers. Each antibody directed to a different marker is conjugated to a detectable molecule, particularly a fluorescent dye that may be distinguished from other fluorescent dyes coupled to other antibodies. A stream of tagged or “stained” cells is passed through a light source that excites the fluorochrome and the emission spectrum from the cells detected to determine the presence of a particular labeled antibody. By concurrent detection of different fluorochromes, also referred to in the art as multicolor fluorescence cell sorting, cells displaying different sets of cell markers may be identified and isolated from other cells in the population. Other FACS parameters, including, by way of example and not limitation, side scatter (SSC), forward scatter (FSC), and vital dye staining (e.g., with propidium iodide) allow selection of cells based on size and viability. FACS sorting and analysis of HSC and related lineage cells is well-known in the art and described in, for example, U.S. Pat. Nos. 5,137,809; 5,750,397; 5,840,580; 6,465,249; Manz et al. (202) Proc. Natl. Acad. Sci. U.S.A. 99:11872-11877; and Akashi et al. (200) Nature 404:193-197. General guidance on fluorescence activated cell sorting is described in, for example, Shapiro (2003) Practical Flow Cytometry, 4th Ed., Wiley-Liss (2003) and Ormerod (2000) Flow Cytometry: A Practical Approach, 3rd Ed., Oxford University Press.

Another method of isolating useful cell populations involves a solid or insoluble substrate to which is bound antibodies or ligands that interact with specific cell surface markers. In immunoadsorption techniques, cells are contacted with the substrate (e.g., column of beads, flasks, magnetic particles, etc.) containing the antibodies and any unbound cells removed. Immunoadsorption techniques may be scaled up to deal directly with the large numbers of cells in a clinical harvest. Suitable substrates include, by way of example and not limitation, plastic, cellulose, dextran, polyacrylamide, agarose, and others known in the art (e.g., Pharmacia Sepharose 6 MB macrobeads). When a solid substrate comprising magnetic or paramagnetic beads is used, cells bound to the beads may be readily isolated by a magnetic separator (see, e.g., Kato and Radbruch (1993) Cytometry 14:384-92). Affinity chromatographic cell separations typically involve passing a suspension of cells over a support bearing a selective ligand immobilized to its surface. The ligand interacts with its specific target molecule on the cell and is captured on the matrix. The bound cell is released by the addition of an elution agent to the running buffer of the column and the free cell is washed through the column and harvested as a homogeneous population. As apparent to the skilled artisan, adsorption techniques are not limited to those employing specific antibodies, and may use nonspecific adsorption. For example, adsorption to silica is a simple procedure for removing phagocytes from cell preparations. One of the most common uses of this technology is for isolating circulating tumor cells (CTCs) from the blood of breast, NSC lung cancer, prostate and colon cancer patients using an antibody against EpCAM, a cell surface glycoprotein that has been found to be highly expressed in epithelial cancers.

FACS and most batch wise immunoadsorption techniques may be adapted to both positive and negative selection procedures (see, e.g., U.S. Pat. No. 5,877,299). In positive selection, the desired cells are labeled with antibodies and removed away from the remaining unlabeled/unwanted cells. In negative selection, the unwanted cells are labeled and removed. Another type of negative selection that may be employed is use of antibody/complement treatment or immunotoxins to remove unwanted cells.

In still other embodiments, microfluidics, one of the newest technologies, is used to isolate cancer cells. This method used a microfluidic chip with a spiral channel that can isolate circulating tumor cells (CTCs) from blood based upon their size. A sample of blood is pumped into the device and as cells flow through the channel at high speeds, the inertial and centrifugal forces cause smaller cells to flow along the outer wall while larger cells, including CTCs, flow along the inner wall. Researchers have used this chip technology to isolate CTCs from the blood of patients with metastatic lung or breast cancer.

Fluorescent nanodiamonds (FNDs), according to a recently published article (Lin et al. Small (2015) 11:4394-4402), can be used to label and isolate slow-proliferating/quiescent cancer stem cells, which, according to study authors, have been difficult to isolate and track over extended time periods using traditional fluorescent markers. It was concluded that nanoparticles do not cause DNA damage or impair cell growth, and that they outperformed EdU and CFSE fluorescent labels in terms of long-term tracking capability.

It is to be understood that the purification or isolation of cells also includes combinations of the methods described above. A typical combination may comprise an initial procedure that is effective in removing the bulk of unwanted cells and cellular material. A second step may include isolation of cells expressing a marker common to one or more of the progenitor cell populations by immunoadsorption on antibodies bound to a substrate. An additional step providing higher resolution of different cell types, such as FACS sorting with antibodies to a set of specific cellular markers, may be used to obtain substantially pure populations of the desired cells.

b. Cancer Cell Engineering and Modification

The cancer cells in the cancer vaccine may have a decreased copy number, amount, and/or activity of at least one biomarker that is important for differentiation of the cancer cells. For example, the cancer cells may have a decreased copy number, amount, and/or activity of at least one biomarker selected from: Cox2, Rag2, NFkB, STAT3, CD133, NEMO, TNF-a, DAP10, DAP12, Clc-5, MCP-1, Transglutaminase 3, Presenilin 1, Presenilin 2, Annexin-1, A20 (TNFAIP3), Galectin-1, Galectin-3, PGC-1a, LDLR, Abca1, Gprc5a, BCMO1, PAP/HIP, ApoE, IL-6, IL-8, GSK3, ADAM17, NKG2A, CD3 zeta, MCT4, c-CBL, EZH2, SUP-1, and Cbl-b. In some embodiments, the copy number, amount, and/or activity of at least one biomarker is decreased in the cancer cells by contacting the cancer cells with a small molecule inhibitor, CRISPR guide RNA (gRNA), RNA interfering agent, antisense oligonucleotide, peptide or peptidomimetic inhibitor, aptamer, antibody, and/or intrabody.

In some embodiments, peptides or peptide mimetics can be used to antagonize the activity of a biomarker of the present disclosure. In some embodiments, variants of a biomarker of the present disclosure which function as a modulating agent for the respective full length protein, can be identified by screening combinatorial libraries of mutants, e.g., truncation mutants, for antagonist activity. In some embodiments, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In some embodiments, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. USA 89:7811-7815; Delagrave et al. (1993) Protein Eng. 6(3):327-331). In some embodiments, cell based assays can be exploited to analyze a variegated polypeptide library. For example, a library of expression vectors can be transfected into a cell line which ordinarily synthesizes a biomarker of the present disclosure. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well-known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides disclosed herein can be used therapeutically to treat disease, e.g., by altering costimulation in a patient.

Peptidomimetics (Fauchere (1986) Adv. Drug Res. 15:29; Veber and Freidinger (1985) TINS p. 392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., (1983) Vega Data Vol. 1, Issue 3, “Peptide Backbone Modifications” (general review); Morley, J. S. (1980) Trends Pharm. Sci. 463-468 (general review); Hudson, D. et al. (1979) Int. J. Pept. Prot. Res. 14:177-185 (—CH₂NH—, CH₂CH₂—); Spatola, A. F. et al. (1986) Life Sci. 38:1243-1249 (—CH₂—S); Hann, M. M. (1982) J. Chem. Soc. Perkin Trans. L 307-314 (—CH—CH—, cis and trans); Almquist, R. G. et al. (1980) J. Med Chem. 23:1392-1398 (—COCH₂—); Jennings-White, C. et al. (1982) Tetrahedron Lett. 23:2533 (—COCH₂—); Szelke, M. et al. (1982) European Appln. EP 45665 CA: 97:39405 (—CH(OH)CH₂—); Holladay, M. W. et al. (1983) Tetrahedron Lett. 24:4401-4404 (—C(OH)CH₂—); and Hruby, V. J. (1982) Life Sci. 31:189-199 (—CH₂—S—); each of which is incorporated herein by reference. A particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivatization (e.g., labeling) of peptidomimetics should not substantially interfere with the desired biological or pharmacological activity of the peptidomimetic.

Also encompassed by the present invention are small molecules which can modulate (e.g., inhibit) activity of a biomarker of the present disclosure or its interaction with its natural binding partners. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad Sci. USA 90:6909; Erb et al. (1994) Proc. Natl. Acad Sci. USA 91:11422; Zuckermann et al. (1994) J. Med Chem. 37:2678; Cho et al. (1993) Science 261:1303; Carrell et al. (1994) Angew. Chem. Int. Ed Engl. 33:2059; Carell et al. (1994) Angew. Chem. Int. Ed Engl. 33:2061; and in Gallop et al. (1994) J. Med Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.). Compounds can be screened in cell based or non-cell based assays. Compounds can be screened in pools (e.g., multiple compounds in each testing sample) or as individual compounds.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., small non-coding RNAS such as miRNAs, pre-miRNAs, pri-miRNAs, miRNA*, anti-miRNA, a miRNA binding site, a variant and/or functional variant thereof, cellular mRNAs or a fragments thereof). In some embodiments, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, a biomarker of the present disclosure. In some embodiments, the small nucleic acids or antisense oligonucleotides or derivatives thereof are small RNAs (e.g., microRNAs) or complements of small RNAs. In other embodiments, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21, 20, 19, 18, 17, 16, 15, or 10 nucleotides in length. In other embodiments, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 2-5, 5-10, 10-20, 10-30 or more nucleic acids comprising or capable of expressing small nucleic acids or antisense oligonucleotides or derivatives thereof.

In some embodiments, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well-known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA (e.g., cancer cell proliferation inhibition, induction of cancer cell apoptosis, enhancement of cancer cell susceptibility to chemotherapeutic agents, specific miRNA target inhibition). In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

MicroRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences of the invention may be associated with a second RNA sequence that may be located on the same RNA molecule or on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH₂, NHCOCH₃, and biotin.

In other embodiments, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

“Piwi-interacting RNA (piRNA)” is the largest class of small non-coding RNA molecules. piRNAs form RNA-protein complexes through interactions with piwi proteins. These piRNA complexes have been linked to both epigenetic and post-transcriptional gene silencing of retrotransposons and other genetic elements in germ line cells, particularly those in spermatogenesis. They are distinct from microRNA (miRNA) in size (26-31 nt rather than 21-24 nt), lack of sequence conservation, and increased complexity. However, like other small RNAs, piRNAs are thought to be involved in gene silencing, specifically the silencing of transposons. The majority of piRNAs are antisense to transposon sequences, suggesting that transposons are the piRNA target. In mammals it appears that the activity of piRNAs in transposon silencing is most important during the development of the embryo, and in both C. elegans and humans, piRNAs are necessary for spermatogenesis. piRNA has a role in RNA silencing via the formation of an RNA-induced silencing complex (RISC).

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, piRNA, miRNA, pre-miRNA, pri-miRNA, miRNA*, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002)Mol. Cell 9:1327-1333; Tuschl (2002), Nat. Biotechnol. 20:446-448; Brummelkamp et al. (2002) Science 296:550-553; Miyagishi et al. (2002) Nat. Biotechnol. 20:497-500; Paddison et al. (2002) Genes Dev. 16:948-958; Lee et al. (2002) Nat. Biotechnol. 20:500-505; and Paul et al. (2002) Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in vivo. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to a gene encoding a biomarker of the present disclosure). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′,3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21, 20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. U.S.A. 84:648-652; PCT Publication No. WO88/09810) or the blood-brain barrier (see, e.g., PCT Publication No. WO89/10134), hybridization-triggered cleavage agents. (See, e.g., Krol et al. (1988) BioTech. 6:958-976) or intercalating agents. (See, e.g., Zon (1988) Pharm. Res. 5:539-549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet other embodiments, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In additional embodiments, small nucleic acids and/or antisense oligonucleotides are α-anomeric oligonucleotides. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-O-methylribonucleotide (Inoue et al. (1987) Nucl. Acids Res. 15:6131-6148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), Cruachem (Glasgow, UK), and Exiqon (Vedbaek, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In some embodiments, small nucleic acids and/or antisense oligonucleotides may comprise or be generated from double stranded small interfering RNAs (siRNAs), in which sequences fully complementary to cellular nucleic acids (e.g. mRNAs) sequences mediate degradation or in which sequences incompletely complementary to cellular nucleic acids (e.g., mRNAs) mediate translational repression when expressed within cells. In other embodiments, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. In vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nature Biotechnology 20:1006; and Brummelkamp et al. (2002) Science 296:550). Vectors that can be used are commercially available, e.g., from OligoEngine under the name pSuper RNAi System™.

Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site-specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well-known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al. (1984) Science 224:574-578; Zaug et al. (1986) Science 231:470-475; Zaug et al. (1986) Nature 324:429-433; WO 88/04300; and Been et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well-known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

Aptamers are oligonucleotide or peptide molecules that bind to a specific target molecule. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Peptide aptamers are artificial proteins selected or engineered to bind specific target molecules. These proteins consist of one or more peptide loops of variable sequence displayed by a protein scaffold. They are typically isolated from combinatorial libraries and often subsequently improved by directed mutation or rounds of variable region mutagenesis and selection. The “Affimer protein”, an evolution of peptide aptamers, is a small, highly stable protein engineered to display peptide loops which provides a high affinity binding surface for a specific target protein. It is a protein of low molecular weight, 12-14 kDa, derived from the cysteine protease inhibitor family of cystatins. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

The present invention also contemplates well-known methods for genetically modifying the genome of an organism or cell to modify the expression and/or activity of a biomarker without contacting the organism or cell with agent once the genetic modification has been completed. For example, cancer cells can be genetically modified using recombinant techniques in order to modulate the expression and/or activity of a biomarker of the present disclosure, such that no agent needs to contact the cancer cells in order for the expression and/or activity a biomarker of the present disclosure to be modulated. For example, targeted or untargeted gene knockout methods can be used, such as to recombinantly engineer subject cancer cell ex vivo prior to infusion into the subject. For example, the target DNA in the genome can be manipulated by deletion, insertion, and/or mutation using retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis. Such methods generally use host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

Similarly, the CRISPR-Cas system can be used for precise editing of genomic nucleic acids (e.g., for creating null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

In some embodiments, the cancer cells are non-replicative. In certain embodiments, the cancer cells are non-replicative due to irradiation (e.g., γ and/or UV irradiation), and/or administration of an agent rendering cell replication incompetent (e.g., compounds that disrupt the cell membrane, inhibitors of DNA replication, inhibitors of spindle formation during cell division, etc.). Typically a minimum dose of about 3500 rads radiation is sufficient, although doses up to about 30,000 rads are acceptable. In some embodiments, a sub-lethal dose of irradiation may be used. For example, the cancer cells may be irradiated to suppress cell proliferation before administration of the cancer vaccine to reduce the risk of giving rise to new neoplastic lesions. It is understood that irradiation is only one way to render the cells non-replicative, and that other methods which result in cancer cells incapable of cell division but that retain the ability to trigger the antitumor immunity are included in the present invention.

### Activation of NK Cells In Vitro or Ex Vivo

In some embodiments, the compositions of the present disclosure are used to activate NK cells in vitro or ex vivo, using a method comprising contacting the NK cell with the cancer vaccine and/or the pharmaceutical composition of the present disclosure. Detailed descriptions are provided in the Examples below. A method of activating NK cells in vitro or ex vivo is also described in Kaur et al. (2020) Cancer 12(63):1-23; Kaur et al. (2018) Current Opinion in Immunology 51:170-180; WO 2018/152340; and WO18/112366.

### Methods for Detection of Copy Number

Methods of evaluating the copy number of a biomarker DNA are well-known to those of skill in the art. The presence or absence of chromosomal gain or loss can be evaluated simply by a determination of copy number of the regions or markers identified herein.

In some embodiments, a biological sample is tested for the presence of copy number changes in genomic loci containing the genomic marker.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In some embodiments, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In some embodiments, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc.). In other embodiments, the hybridization protocol of Pinkel et al. (1998) Nature Genetics 20: 207-211, or of Kallioniemi (1992) Proc. Natl Acad Sci USA 89:5321-5325 (1992) is used.

In still other embodiments, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). Measurement of DNA copy number at microsatellite loci using quantitative PCR analysis is described in Ginzonger et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods encompassed by the present invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren et al. (1988) Science 241:1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Loss of heterozygosity (LOH) and major copy proportion (MCP) mapping (Wang, Z. C. et al. (2004) Cancer Res 64(1):64-71; Seymour, A. B. et al. (1994) Cancer Res 54, 2761-4; Hahn, S. A. et al. (1995) Cancer Res 55, 4670-5; Kimura, M. et al. (1996) Genes Chromosomes Cancer 17, 88-93; Li et al., (2008) MBC Bioinform. 9, 204-219) may also be used to identify regions of amplification or deletion.

### Methods for Detection of Biomarker Expression and Amount

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In other embodiments, detecting or determining expression levels of a biomarker and functionally similar homologs thereof, including a fragment or genetic alteration thereof (e.g., in regulatory or promoter regions thereof) comprises detecting or determining RNA levels for the marker of interest. In some embodiments, one or more cells from the subject to be tested are obtained and RNA is isolated from the cells. In some embodiments, a sample of tissue cells is obtained from the subject.

In some embodiments, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded.

Accordingly, in preferred embodiments, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In some embodiments, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, NY).

In certain preferred embodiments, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope encompassed by the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall et al., PCR Methods and Applications 4: 80-84 (1994). Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos. 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In some embodiments, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In some embodiments, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In other embodiments, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In certain embodiments, the biological sample contains polypeptide molecules from the test subject. Alternatively, the biological sample can contain mRNA molecules from the test subject or genomic DNA molecules from the test subject.

In other embodiments, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

### Methods for Detection of Biomarker Amount or Activity

The activity or level of a biomarker protein can be detected and/or quantified by detecting or quantifying the expressed polypeptide. The polypeptide can be detected and quantified by any of a number of means well-known to those of skill in the art. Decreased levels of polypeptide expression of the polypeptides encoded by a biomarker nucleic acid and functionally similar homologs thereof, including a fragment or genetic alteration thereof (e.g., in regulatory or promoter regions thereof) are associated with the de-differentiation of cells (e.g., cancer cells). Any method known in the art for detecting polypeptides can be used.

Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as ¹²I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In some embodiments, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzymes are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art. As described above, antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

### Methods for Detection of Biomarker Structural Alterations

The following illustrative methods can be used to identify the presence of a structural alteration in a biomarker nucleic acid and/or biomarker polypeptide molecule in order to, for example, identify one or more biomarkers listed in Table 1, or other biomarkers described herein.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In alternative embodiments, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet other embodiments, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In preferred embodiments, the control DNA or RNA can be labeled for detection.

In still other embodiments, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to exemplary embodiments, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In preferred embodiments, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In further embodiments, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

### Methods of Treatment

The present invention provides for both prophylactic and therapeutic methods of treating a subject at risk of (or susceptible to) a cancer. The cancer may be a solid or hematological cancer. In some embodiments, the cancer is of the same cancer type as the cancer vaccine. In other embodiments, the cancer is of a different cancer type than the cancer vaccine.

a. Prophylactic Methods

In certain aspects, the present invention provides a method of preventing a cancer in a subject, by administering to the subject a) a therapeutically effective amount of the cancer vaccine or the pharmaceutical composition of the present disclosure; and/or b) a therapeutically effective number of NK cells activated by contacting the NK cells with the cancer vaccine or the pharmaceutical composition of the present disclosure. Administration of a prophylactic agent (e.g., the cancer vaccine described herein) can occur prior to the manifestation of symptoms characteristic of cancer, such that a cancer is prevented or, alternatively, delayed in its progression. In certain embodiments, administration of the prophylactic agent (e.g., the cancer vaccine described herein) protects the subject from recurrent cancer.

b. Therapeutic Methods

Another aspects of the present invention pertain to methods treating a cancer in a subject comprising administering to the subject a) a therapeutically effective amount of the cancer vaccine or the pharmaceutical composition of the present disclosure; and/or b) a therapeutically effective number of NK cells activated by contacting the NK cells with the cancer vaccine or the pharmaceutical composition of the present disclosure. The cancer cells of the cancer vaccine will have an immunocompatibility relationship to the subject and any such relationship is contemplated for use according to the present invention. For example, the cancer cells can be syngeneic. The term “syngeneic” can refer to the state of deriving from, originating in, or being members of the same species that are genetically identical, particularly with respect to antigens or immunological reactions. These include identical twins having matching MHC types. Thus, a “syngeneic transplant” refers to transfer of cells from a donor to a recipient who is genetically identical to the donor or is sufficiently immunologically compatible as to allow for transplantation without an undesired adverse immunogenic response (e.g., such as one that would work against interpretation of immunological screen results described herein).

A syngeneic transplant can be “autologous” if the transferred cells are obtained from and transplanted to the same subject. An “autologous transplant” refers to the harvesting and reinfusion or transplant of a subject's own cells or organs. Exclusive or supplemental use of autologous cells may eliminate or reduce many adverse effects of administration of the cells back to the host, particular graft versus host reaction.

A syngeneic transplant can be “matched allogeneic” if the transferred cells are obtained from and transplanted to different members of the same species yet have sufficiently matched major histocompatibility complex (MHC) antigens to avoid an adverse immunogenic response. Determining the degree of MHC mismatch may be accomplished according to standard tests known and used in the art. For instance, there are at least six major categories of MHC genes in humans, identified as being important in transplant biology. HLA-A, HLA-B, HLA-C encode the HLA class I proteins while HLA-DR, HLA-DQ, and HLA-DP encode the HLA class II proteins. Genes within each of these groups are highly polymorphic, as reflected in the numerous HLA alleles or variants found in the human population, and differences in these groups between individuals is associated with the strength of the immune response against transplanted cells. Standard methods for determining the degree of MHC match examine alleles within HLA-B and HLA-DR, or HLA-A, HLA-B and HLA-DR groups. Thus, tests may be made of at least 4, and even 5 or 6 MHC antigens within the two or three HLA groups, respectively. In serological MHC tests, antibodies directed against each HLA antigen type are reacted with cells from one subject (e.g., donor) to determine the presence or absence of certain MHC antigens that react with the antibodies. This is compared to the reactivity profile of the other subject (e.g., recipient). Reaction of the antibody with an MHC antigen is typically determined by incubating the antibody with cells, and then adding complement to induce cell lysis (i.e., lymphocytotoxicity testing). The reaction is examined and graded according to the amount of cells lysed in the reaction (see, for example, Mickelson and Petersdorf (1999) Hematopoietic Cell Transplantation, Thomas, E. D. et al. eds., pg 28-37, Blackwell Scientific, Malden, Mass.). Other cell-based assays include flow cytometry using labeled antibodies or enzyme linked immunoassays (ELISA). Molecular methods for determining MHC type are well-known and generally employ synthetic probes and/or primers to detect specific gene sequences that encode the HLA protein. Synthetic oligonucleotides may be used as hybridization probes to detect restriction fragment length polymorphisms associated with particular HLA types (Vaughn (2002) Method. Mol. Biol. MHC Protocol. 210:45-60). Alternatively, primers may be used for amplifying the HLA sequences (e.g., by polymerase chain reaction or ligation chain reaction), the products of which may be further examined by direct DNA sequencing, restriction fragment polymorphism analysis (RFLP), or hybridization with a series of sequence specific oligonucleotide primers (SSOP) (Petersdorf et al. (1998) Blood 92:3515-3520; Morishima et al. (2002) Blood 99:4200-4206; and Middleton and Williams (2002) Method. Mol. Biol. MHC Protocol. 210:67-112).

A syngeneic transplant can be “congenic” if the transferred cells and cells of the subject differ in defined loci, such as a single locus, typically by inbreeding. The term “congenic” refers to deriving from, originating in, or being members of the same species, where the members are genetically identical except for a small genetic region, typically a single genetic locus (i.e., a single gene). A “congenic transplant” refers to transfer of cells or organs from a donor to a recipient, where the recipient is genetically identical to the donor except for a single genetic locus. For example, CD45 exists in several allelic forms and congenic mouse lines exist in which the mouse lines differ with respect to whether the CD45.1 or CD45.2 allelic versions are expressed.

By contrast, “mismatched allogeneic” refers to deriving from, originating in, or being members of the same species having non-identical major histocompatibility complex (MHC) antigens (i.e., proteins) as typically determined by standard assays used in the art, such as serological or molecular analysis of a defined number of MHC antigens, sufficient to elicit adverse immunogenic responses. A “partial mismatch” refers to partial match of the MHC antigens tested between members, typically between a donor and recipient. For instance, a “half mismatch” refers to 50% of the MHC antigens tested as showing different MHC antigen type between two members. A “full” or “complete” mismatch refers to all MHC antigens tested as being different between two members.

Similarly, in contrast, “xenogeneic” refers to deriving from, originating in, or being members of different species, e.g., human and rodent, human and swine, human and chimpanzee, etc. A “xenogeneic transplant” refers to transfer of cells or organs from a donor to a recipient where the recipient is a species different from that of the donor.

In addition, cancer cells can be obtained from a single source or a plurality of sources (e.g., a single subject or a plurality of subjects). A plurality refers to at least two (e.g., more than one). In still other embodiments, the non-human mammal is a mouse. The animals from which cell types of interest are obtained may be adult, newborn (e.g., less than 48 hours old), immature, or in utero. Cell types of interest may be primary cancer cells, cancer stem cells, established cancer cell lines, immortalized primary cancer cells, and the like. In certain embodiments, the immune systems of host subjects can be engineered or otherwise selected to be immunological compatible with transplanted cancer cells. For example, in some embodiments, the subject may be “humanized” in order to be compatible with human cancer cells. The term “immune-system humanized” refers to an animal, such as a mouse, comprising human HSC lineage cells and human acquired and innate immune cells, survive without being rejected from the host animal, thereby allowing human hematopoiesis and both acquired and innate immunity to be reconstituted in the host animal. Acquired immune cells include T cells and B cells. Innate immune cells include macrophages, granulocytes (basophils, eosinophils, neutrophils), DCs, NK cells and mast cells. Representative, non-limiting examples include SCID-hu, Hu-PBL-SCID, Hu-SRC-SCID, NSG (NOD-SCID IL2r-gamma(null) lack an innate immune system, B cells, T cells, and cytokine signaling), NOG (NOD-SCID IL2r-gamma(truncated)), BRG (BALB/c-Rag2(null)IL2r-gamma(null)), and H2dRG (Stock-H2d-Rag2(null)IL2r-gamma(null)) mice (see, for example, Shultz et al. (2007) Nat. Rev. Immunol. 7:118; Pearson et al. (2008) Curr. Protocol. Immunol. 15:21; Brehm et al. (2010) Clin. Immunol. 135:84-98; McCune et al. (1988) Science 241:1632-1639, U.S. Pat. No. 7,960,175, and U.S. Pat. Publ. 2006/0161996), as well as related null mutants of immune-related genes like Rag1 (lack B and T cells), Rag2 (lack B and T cells), TCR alpha (lack T cells), perforin (cD8+ T cells lack cytotoxic function), FoxP3 (lack functional CD4+ T regulatory cells), IL2rg, or Prf1, as well as mutants or knockouts of PD-1, PD-L1, Tim3, and/or 2B4, allow for efficient engraftment of human immune cells in and/or provide compartment-specific models of immunocompromised animals like mice (see, for example, PCT Publ. WO2013/062134). In addition, NSG-CD34+(NOD-SCID IL2r-gamma(null) CD34+) humanized mice are useful for studying human gene and tumor activity in animal models like mice.

As used herein, “obtained” from a biological material source means any conventional method of harvesting or partitioning a source of biological material from a donor. For example, biological material may obtained from a solid tumor, a blood sample, such as a peripheral or cord blood sample, or harvested from another body fluid, such as bone marrow or amniotic fluid. Methods for obtaining such samples are well-known to the artisan. In the present invention, the samples may be fresh (i.e., obtained from a donor without freezing). Moreover, the samples may be further manipulated to remove extraneous or unwanted components prior to expansion. The samples may also be obtained from a preserved stock. For example, in the case of cell lines or fluids, such as peripheral or cord blood, the samples may be withdrawn from a cryogenically or otherwise preserved bank of such cell lines or fluid. Such samples may be obtained from any suitable donor.

The obtained populations of cells may be used directly or frozen for use at a later date. A variety of mediums and protocols for cryopreservation are known in the art. Generally, the freezing medium will comprise DMSO from about 5-10%, 10-90% serum albumin, and 50-90% culture medium. Other additives useful for preserving cells include, by way of example and not limitation, disaccharides such as trehalose (Scheinkonig et al. (2004) Bone Marrow Transplant. 34:531-536), or a plasma volume expander, such as hetastarch (i.e., hydroxyethyl starch). In some embodiments, isotonic buffer solutions, such as phosphate-buffered saline, may be used. An exemplary cryopreservative composition has cell-culture medium with 4% HSA, 7.5% dimethyl sulfoxide (DMSO), and 2% hetastarch. Other compositions and methods for cryopreservation are well-known and described in the art (see, e.g., Broxmeyer et al. (2003) Proc. Natl. Acad. Sci. U.S.A. 100:645-650). Cells are preserved at a final temperature of less than about −135° C.

c. Combination Therapy

The therapeutic agents of the present invention can be used alone or can be administered in combination therapy with, e.g., chemotherapeutic agents, hormones, antiangiogens, radiolabeled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, agents of the present invention can be administered with a therapeutically effective dose of chemotherapeutic agent. In other embodiments, agents of the present invention are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, TLT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the cancer vaccine is administered in combination with one or more inhibitors of immune checkpoints, such as PD1, PD-L1, and/or CD47 inhibitors.

Adoptive cell-based immunotherapies can be combined with the therapies of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C(indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereof, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an TL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, agents and therapies other than immunotherapy or in combination thereof can be used with in combination with an anti-KHK antibodies to treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

The immunotherapy and/or cancer therapy may be administered before, after, or concurrently with the cancer vaccine described herein. The duration and/or dose of treatment with the cancer vaccine may vary according to the particular cancer vaccine, or the particular combinatory therapy. An appropriate treatment time for a particular cancer therapeutic agent will be appreciated by the skilled artisan. The invention contemplates the continued assessment of optimal treatment schedules for each cancer therapeutic agent, where the phenotype of the cancer of the subject as determined by the methods of the invention is a factor in determining optimal treatment doses and schedules.

### Clinical Efficacy

Clinical efficacy can be measured by any method known in the art. For example, the response to a therapy (e.g., a cancer vaccine comprising cancer cells, engineered monocytes and/or osteoclasts, or activated NK cells), relates to e.g., any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al. (2007) J. Clin. Oncol. 25:4414-4422) or Miller-Payne score (Ogston et al. (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer vaccine therapeutic regimen is at least 25%, 30%, 35%, 40%, 45%, 50%, 55%, 60%, 65%, 70%, 75%, 80%, 85%, or more.

Additional criteria for evaluating the response to a therapy (e.g., a cancer vaccine comprising cancer cells, engineered monocytes and/or osteoclasts, or activated NK cells) are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular agent encompassed by the present invention can be administered to a population of subjects and the outcome can be correlated to biomarker measurements that were determined prior to administration of a therapy (e.g., a cancer vaccine comprising cancer cells, engineered monocytes and/or osteoclasts, or activated NK cells). The outcome measurement may be pathologic response to therapy given in the neoadjuvant setting. Alternatively, outcome measures, such as overall survival and disease-free survival can be monitored over a period of time for subjects following a therapy (e.g., a cancer vaccine comprising cancer cells, engineered monocytes and/or osteoclasts, or activated NK cells). In certain embodiments, the same doses of the agent are administered to each subject. In related embodiments, the doses administered are standard doses known in the art for the agent. The period of time for which subjects are monitored can vary. For example, subjects may be monitored for at least 2, 4, 6, 8, 10, 12, 14, 16, 18, 20, 25, 30, 35, 40, 45, 50, 55, or 60 months.

### Pharmaceutical Compositions and Administration

For cancer vaccines of the present invention, cancer cells can be administered at 1, 10, 1000, 10,000, 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, 1.0×10⁹ or more, or any range in between or any value in between, cells per kilogram of subject body weight. The number of cells transplanted may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiments, transplantation of at least about 100, 1000, 10,000, 0.1×10⁶, 0.5×10⁶, 1.0×10⁶, 2.0×10⁶, 3.0×10⁶, 4.0×10⁶, or 5.0×10⁶ total cells relative to an average size mouse is effective.

Cancer vaccines can be administered in any suitable route as described herein, such as by infusion. Cancer vaccines can also be administered before, concurrently with, or after, other anti-cancer agents.

Administration can be accomplished using methods generally known in the art. Agents, including cells, may be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, subcutaneous, intratumorally, or intramuscular administration. For example, subjects of interest may be engrafted with the transplanted cells by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. In certain embodiments, the cancer vaccine of the present invention is injected to the subject intratumorally or subcutaneously. Cells may be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427).

Two or more cell types can be combined and administered, such as cancer vaccine and adoptive cell transfer of stem cells, cancer vaccine and other cell-based vaccines, and the like. For example adoptive cell-based immunotherapies can be combined with the cancer vaccine of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like. The ratio of cancer cells in the cancer vaccine described herein to other cell types can be 1:1, but can modulated in any amount desired (e.g., 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

Engraftment of transplanted cells may be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, 28 days or can signal the time for tumor harvesting. Any such metrics are variables that can be adjusted according to well-known parameters in order to determine the effect of the variable on a response to anti-cancer immunotherapy. In addition, the transplanted cells can be co-transplanted with other agents, such as cytokines, extracellular matrices, cell culture supports, and the like. In addition, anti-cancer agents (e.g., a cancer vaccine comprising cancer cells, engineered monocytes and/or osteoclasts, or activated NK cells) of the present invention can be administered to subjects or otherwise applied outside of a subject body in a biologically compatible form suitable for pharmaceutical administration. By “biologically compatible form suitable for administration in vivo” is meant a form to be administered in which any toxic effects are outweighed by the therapeutic effects. Administration of an anti-cancer agent as described herein can be in any pharmacological form including a therapeutically active amount of an agent alone or in combination with a pharmaceutically acceptable carrier. The phrase “therapeutically-effective amount” as used herein means that amount of an agent that is effective for producing some desired therapeutic effect, e.g., cancer treatment, at a reasonable benefit/risk ratio.

Administration of a therapeutically active amount of the therapeutic composition of the present invention is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

A combination dosage form or simultaneous administration of single agents can result in effective amounts of each desired modulatory agent present in the patient at the same time.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

The agent may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of agents suitable for injectable use include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an agent of the invention in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the agent plus any additional desired ingredient from a previously sterile-filtered solution thereof.

When the agent is suitably protected, as described above, the protein can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well-known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form”, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by, and directly dependent on, (a) the unique characteristics of the active compound and the particular therapeutic effect to be achieved, and (b) the limitations inherent in the art of compounding such an active compound for the treatment of sensitivity in individuals.

### Kits

The present invention also encompasses kits. For example, the kit can comprise a cancer vaccine of the present disclosure, a monocyte and/or an osteoclast engineered as described herein, an NK cell activated using the methods and compositions described herein, any one of pharmaceutical compositions described herein, a combination therapy including a bacterial composition described herein, or any combination thereof, packaged in a suitable container and can further comprise instructions for using such reagents. The kit may also contain other components, such as administration tools packaged in a separate container.

Other embodiments of the present invention are described in the following Examples. The present invention is further illustrated by the following examples which should not be construed as further limiting. The contents of all references, patents and published patent applications cited throughout this application, as well as the Figures, are incorporated herein by reference.

### EXAMPLES

### Example 1: Materials and Methods

### Cox2 Mice

Myeloid cell-specific COX-2 targeted knockout mice (Cox-2flox/flox;LysMCre/+) and their control wild type (WT) littermates (Cox-2flox/flox;LysM+/+), as well as global COX-2 knockout (COX-2−/−) and their control wild type littermates were generated and bred at UCLA in Dr. Harvey Herschman's laboratory and used for this study. C57BL/6 mice were purchased from Jackson Laboratory (Bar Harbor, ME, USA).

### Cell Lines, Reagents, and Antibodies

RPMI 1640 supplemented with 10% Fetal Bovine Serum (FBS) was used for the cultures of human NK cells, monocytes, and mouse NK cells, T cells, monocytes and DCs. RPMI 1640 supplemented with 10% FBS was also used to culture mouse T cell lymphoma (YAC-1). ST63 cells were cultured in RPMI 1640 supplemented with 10% FBS. COX-2 wild type and COX-2 knockout Mouse Embryonic Fibroblasts (MEFs) were cultured in DMEM supplemented with 10% FBS. Oral Squamous Cancer Stem Cells (OSCSCs) were isolated from the tongue tumors of the patients at UCLA and cultured in RPMI 1640 supplemented with 10% FBS (Gemini Bio-Products, CA), 1.4% antibiotic antimycotic, 1% sodium pyruvate, 1.4% non-essential amino acids, 1% L-glutamine, 0.2% gentamicin (Gemini Bio-Products, CA, USA) and 0.15% sodium bicarbonate (Fisher Scientific, PA, USA). IFN-γ was purchased from Biolegend (San Diego, CA, USA) and TNF-α was purchased from PeproTech (Rocky Hill, NJ, USA). LPS was purchased from Sigma-Aldrich (St. Louis, MO). IL-4 and GMCSF were purchased from Biolegend (San Diego, CA, USA) and used to differentiate purified monocytes into DCs. Recombinant IL-2 was obtained from NIH-BRB. Antibodies to CD16, B7H1, CD45, CD54, DX5, Ly49A, Ly49D, Rae-17, NKG2D, and F4/80 were purchased from Biolegend (San Diego, CA, USA). Antibody to MHC class-I was purchased from eBioscience (San Diego, CA, USA). Flow cytometry analysis was performed using Beckman Coulter Epics XL cytometer (Brea, CA, USA) and results were analyzed in FlowJo vX software (Tree Star, Ashland, OR, USA). The mouse and human NK cells, T cells, and monocyte purification kits were obtained from Stem Cell Technologies (Vancouver, BC, Canada).

### Bacterial Preparation

AJ2 is a combination of eight gram-positive bacterial strains (Streptococcus thermophilus, Bifidobacterium longum, Bifidobacterium breve, Bifidobacterium infantis, Lactobacillus acidophilus, Lactobacillus Plantarum, Lactobacillus casei, and Lactobacillus bulgaricus) each selected and combined for the optimal capability to induce differentiation of stem cells. AJ2 was re-suspended in RPMI supplemented with 10% FBS (Gemini Bio-Products, CA) at a final concentration of 10 mg/mL. The bacteria were then sonicated using ultra-sonicator for 15 s while on ice. Afterward, the sonicated bacteria were incubated for 30 s on ice. The sonication process was repeated 20 times to achieve complete sonication. Lastly, the sonicated samples (sAJ2) were aliquoted and stored in −80° C. freezer until use.

### Purification of Human NK Cells and Monocytes

Written informed consents approved by UCLA Institutional Review Board (IRB) were obtained from the blood donors and all the procedures were approved by the UCLA-IRB. NK cells from healthy donors were isolated as described in the art. Briefly, peripheral blood lymphocytes were obtained after Ficoll-hypaque centrifugation and purified NK cells were negatively selected by using an NK cell isolation kit (Stem Cell Technologies, Vancouver, BC, Canada). The purity of NK cell population was found to be >90% based on flow cytometric analysis of anti-CD16 antibody stained cells. The levels of contaminating CD3+ T cells remained low, at 2.4+/−1%, similar to that obtained by the nonspecific staining using isotype control antibody throughout the experimental procedures. The adherent subpopulation of PBMCs was detached from the tissue culture plates and monocytes were purified using isolation kit obtained from Stem Cell Technologies (Vancouver, BC, Canada). Greater than 95% purity was achieved based on flow cytometric analysis of CD14 antibody stained monocytes.

### Mouse NK Cells, T Cells, Monocytes and Dendritic Cell Cultures

All animal work performed was based on the guidelines established and approved by UCLA Office of Animal Research Oversight. Single cell preparations of mouse splenocytes were used to negatively select mouse NK cells using mouse NK isolation kit purchased from Stem Cell Technologies (Vancouver, Canada). The purity of mouse NK cells were >90% based on staining with PE-conjugated DX5 antibody. NK cells were treated with IL-2 (1×10⁴ U/million NK cells) for 7 days before the cells were used for experiments. T cells were purified using mouse T cell isolation kit purchased from Stem Cell Technologies (Vancouver, BC, Canada). Bone marrow cells were isolated by flushing femurs with PBS supplemented with 2% heat-inactivated FBS. Murine monocytes were then purified from bone marrow cells using monocyte isolation kit obtained from Stem Cell Technologies (Vancouver, BC, Canada). The purity of monocytes was between 86 and 96% based on staining with PE-conjugated anti-CD14 antibody. To differentiate mouse DCs from purified monocytes, IL-4 (20 ng/mL) and GM-CSF (20 ng/mL) were added to monocytes for 7 days.

### ZOL and NaCl Injections in WT Mice and Rag2^{-/-} Mice

7-week old female WT mice, and female Rag2^{-/-} mice (B6(Cg)-Rag2tm1.1Cgn/J null mutation in recombination-activating gene-2 resulting in the deficiency of B and T lymphocytes) were purchased from the Jackson Laboratory, Bar Harbor, ME. The UCLA Animal Research Committee reviewed and approved all experimental protocols involving animals (ARC 1997-136). Female WT mice and Rag2^{-/-} mice were injected with the single injection of 500 μg/kg ZOL or 0.9% NaCl vehicle solution via tail vein.

### Maxillary First Molar Extraction in WT Mice and Rag2^{-/-} Mice

After 1-week of the ZOL and NaCl injection maxillary first molar was extracted as described previously. During the extraction procedure, mice were anesthetized via continuous inhalation of 2-4% isoflurane mixed with oxygen and placed on a custom-made surgical table in a supine position using the fixed positioner on the maxillary incisors. Immediately prior to tooth extraction, 5.0 mg/kg carprofen was subcutaneously injected, and mice received 5.0 mg/kg carprofen injection every 24 h for 48 h. Mice were sacrificed and various tissues were studied 4-weeks after the tooth extraction.

Tissue Collection from WT and Rag2^{-/-} Mice

5-weeks after the mice were received; they were euthanized followed by femur, spleen, pancreas, peri-pancreatic adipose tissue and oral gingiva tissue collection. Femur was cut from both sides and flushed with 1% FBS+PBS to isolate bone marrow cells. Spleen was sliced into small pieces and was passed through 70 μm cell strainer followed by twice with RPMI 1640 media supplemented with 10% antibiotic-antimycotic (Life Technologies, CA). The pancreas, adipose and oral gingival tissue were immediately cut into 1 mm³ pieces and re-suspended in cell-dissociation buffer containing 1 mg/ml collagenase (collagenase IV for pancreas and adipose, and collagenase II for oral gingival tissue), 10 U/ml DNAse I, and 1% bovine serum albumin (BSA) in DMEM and incubated for 20 minutes at 37° C. oven with on a 150-rpm shaker. Samples were then filtered through a 40 μm cell strainer and centrifuged at 1500 rpm for 10 minutes at 4° C. The pellet was re-suspended in RPMI 1640 media supplemented with 10% antibiotic-antimycotic and cells were counted.

### Surface Staining

Flow antibodies used in this study were purchased from Biolegend, San Diego, CA. Freshly isolated cells from each tissue from WT and Rag2^{-/-} female mice were washed twice using ice-cold PBS with 1% BSA. Cells were re-suspended in 50 μl (1×10⁴ cells/50 μl) of cold-1% BSA+PBS and predetermined optimal concentrations of specific mouse monoclonal antibodies were added and cells were incubated on ice for 30 mins. After 30 mins incubation, cells were washed using cold-1% BSA+PBS and brought to 500 μl with 1% BSA+PBS. Surface staining analysis was performed as described previously and flow cytometry analysis was performed using Beckman Coulter Epics XL cytometer (Brea, CA) and results were analyzed in FlowJo vX software (Ashland, OR).

NK Cells Isolations from Splenocytes of WT and Rag2 Mice

NK cells were isolated from splenocytes using mouse NK cells isolation kit (Stem-cells Technologies, Canada). The purity of NK cells was greater than 90% based on flow cytometry analysis of DX5 antibodies.

### Enzyme-Linked Immunosorbent Assay (ELISA) for IFN-γ & Multiplex Assays

Single cell-suspension from each tissue of WT and Rag2^{-/-} female mice were activated with IL-2 (10,000 U/ml) for 5 days before the supernatants/culture media were harvested after to determine the levels of IFN-γ secretion. IFN-γ ELISAs were performed and standard curve was generated as described previously. A standard curve was generated by either two or three-fold dilution of recombinant IFN-γ provided by the manufacturer to analyze and obtain the IFN-γ concentration (Biolegend, San Diego, CA).

Fluorokine MAP cytokine multiplex kits were purchased from R&D Systems (Minneapolis, MN, USA) and the procedures were conducted as suggested by the manufacturer. To analyze and obtain the cytokine and chemokine concentration, a standard curve was generated by either two- or threefold dilution of recombinant cytokines provided by the manufacturer. Analysis was performed using the Star Station software. Samples were analyzed using Beckman Coulter EPICS XL cytometer and subsequently analyzed in FlowJo software (Tree Star, Ashland, OR, USA).

⁵¹Cr Release Cytotoxicity Assay

The standard 4-hour ⁵¹Cr release assay was performed as demonstrated previously. Single cell-suspension from each tissue and spleen-purified NK cells from WT and Rag2^{-/-} mice female mice were activated with IL-2 (10,000 U/ml) for 5 days and were used as effector cells on day 5. Briefly, different numbers of effector cells were incubated with ⁵¹Cr-labeled ST63 target cells for 4 hours, ST63 were previously used as specific targets of NK cells. After that, the supernatants were harvested from each sample and released radioactivity was counted using the gamma counter. The percentage (%) specific cytotoxicity was calculated as follows:

% Cytotoxicity=Experimental cpm−spontaneous cpm/Total cpm−spontaneous cpm
Lytic Units (LU) 30/10⁶ was calculated by using the inverse of the number of effector cells needed to lyse 30% of tumor target ST63 cells×100.

### Statistical Analysis

One-way ANOVA using Turkey's multiple comparison tests was used to compare different groups using Prism 7 software for the statistical analysis. The following symbols represent the levels of statistical significance within each analysis, ***(p value <0.001), **(p value 0.001-0.01), *(p value 0.01-0.05).

### Example 2: Increased Function of NK Cells in Gene Knockout/Down Mice; Cellular De-Differentiation Activates NK Cells

A list is provided herein of cellular genes that augment NK cell function when deleted or decreased in tumors (Table 1). Table 1 provides a short list of genes, which upon deletion in cells trigger inflammation and augment immune cell function in mice and in in vitro culture models. Specifically, the deletion of NF-κB in tumors was found to increase NK cell-mediated cytotoxicity and secretion of IFN-γ significantly, and induce auto-immunity and inflammation in vivo. Moreover, conditional knockout of STAT3 in hematopoietic cells was found to result in the induction of colitis in mice due to chronic gut inflammation. Knockdown of CD44 in breast and melanoma tumors were also able to increase expansion and functional activation of NK cells significantly. In addition, targeted knockdown of COX2 in non-transformed healthy myeloid cells and mouse embryonic fibroblasts were found to increase expansion, and functional activation of NK cells significantly.

Since increased CD44 expression correlated fairly consistently with the poor differentiation or stem-like phenotype of tumor cells, CD44 knocked down tumor cells were used herein to assess their susceptibility to NK cell mediated cytotoxicity. CD44 was knocked down in human breast cancer cell line MDA-MB231 (MDA-MB231shCD44) and human melanoma cell line A375 (A375shCD44) as described in the art. Cells stably expressing shRNA against firefly luciferase were used as controls (MDA-MB231shLUC and A375shLUC). CD44 knockdown and control cell lines were used as target cells against NK cells. CD44 receptor is a cell surface glycoprotein with the ability to bind to other cells and to the extracellular matrix through the hyaluronic acid (HA) binding domain within its amino-terminal ectodomain. Thus, knockdown of CD44 was expected to have profound effect in inhibition of NK function since it has been shown to facilitate NK binding to tumor cells. Surprisingly, MDA-MB23shCD44 cells became more susceptible to NK cell-mediated lysis than MDA-MB231shLUC. Similar to other knockdown tumor models, CD44 knockdown in MDA-MB231 tumors also resulted in a down modulation of CD54, B7H1 and MHC-class I surface receptors. In addition, as shown previously, MDA-MB231shCD44 treated with recombinant interferon (rIFN)-γ were much more resistant to NK cell mediated lysis as compared to untreated MDA-MB231shCD44, and the fold increase in resistance against IL-2-treated NK cell-mediated lysis by the addition of IFN-γ to the cells was greater in MDA-MB231shCD44 than MDA-MB231shLUC when compared to their respective untreated tumors. Similarly, A375shCD44 melanoma cells were more susceptible to NK cell mediated cytotoxicity when compared to A375shLUC tumor cells. A decrease in NK cell susceptibility was observed when either NK supernatants or rIFN-7 were added to A375shLUC or A375shCD44 cells, and, similarly, the fold induction of resistance to NK cell mediated cytotoxicity was much greater in A375shCD44 cells treated with rIFN-7 as compared to A375shLUC cells treated with rIFN-7. The addition of NK supernatants or rIFN-7 to A375shCD44 cells also induced higher fold increase in the surface expression of CD54, B7H1 and MHC-class I when compared to their untreated controls or compared to A375shLUC treated with rIFN-7. Such observations were most intriguing because they were pointing to the fact that even though there is a direct correlation between the stage of differentiation with the susceptibility to NK cell mediated effects, the extent of differentiation induced by the NK cells in poorly differentiated tumors vs. moderately or well differentiated tumors may not be of a linear relationship in regards to the susceptibility to the NK cells or increased expression of surface receptors. The lower the levels of differentiation as assessed by the surface receptor expression and susceptibility to NK cells, the higher will be the induced fold expression of surface receptors associated with differentiation or the extent of resistance in tumor cells. However, in moderately or well differentiated tumors the fold increase levels of surface receptor expression or the extent of resistance in tumor cells will be relatively lower. Accordingly, the more undifferentiated a tumor is, the higher the degree of activation of the NK cells and the greater ability of the cells to release cytokines in order to increase differentiation in the tumors, whereas the higher the differentiation of the tumors, the lower capacity to activate NK cells, which results in an incremental increase in the differentiation of tumors.

One common underlying mechanism for the activation of NK cells in the gene knockout studies was found to be related to the down-modulation of MHC-class I expression on both transformed and non-transformed healthy cells. Surprisingly, hyper-responsiveness of the NK cells were also seen in mice with knockouts of genes that mediate inflammation, in particular even those that are involved in NK cell signaling and activation, such as DAP10/DAP12, indicating that NK cell activation is much more complex than previously envisioned, involving many genes/pathways, and is likely dependent on the modulation of the stage of the differentiation of the cells by these genes/pathways. Therefore, such increases in responsiveness of NK cells when key cellular genes were knocked out or knocked down in interacting cells/tumors may point to the fundamental function of NK cells in targeting cells that lose ability to differentiate optimally, and that the degree of differentiation of the cells is likely the key in regulating the NK cell expansion and function.

TABLE 1
Biomarker knockouts that mediate enhancement of
NK cell function and inflammation

| Gene (−/−) | Gene (−/−) |
|---|---|
| NF-kB (−/−) | Gprc5a (−/−) |
| STAT3 (−/−) | BCMO1 (−/−) |
| CD133 (−/−) | PAP/HIP (−/−) |
| NEMO (−/−) | ApoE (−/−) |
| TNF-α (−/−) | IL-6 (−/−) |
| DAP10/DAP12 (−/−) | IL-8 (−/−) |
| Clc-5 (−/−) | GSK3 (−/−) |
| MCP-1 (−/−) | ADAM17 (−/−) |
| Transglutaminase 3 (−/−) | NKG2A (−/−) |
| Presenilins 1 and 2 (−/−) | CD3ζ (−/−) |
| Annexin-1 (−/−) | MCT4 (−/−) |
| A20 (TNFAIP3) (−/−) | c-CBL (−/−) |
| Galectin-1 (−/−) | EZH2 (−/−) |
| Galectin-3 (−/−) | SHP-1 (−/−) |
| PGC-1α (−/−) | Cb1-b (−/−) |
| LDLR (−/−) | COX-2 (−/−) |
| Abcal (−/−) | Rag2 (−/−) |

*Abca, ATP-binding membrane cassette transporter A1;
ADAM, a disintegrin and metallopeptidase domain;
Apo, apolipoprotein;
BCMO1, β-carotene monooxygenase;
Cbl-b, Casitas B-lineage lymphoma proto-oncogene b;
c-CBL, Casitas B-lineage lymphoma;
CD, cluster of differentiation;
Clc, Charcot-Leyden crystal galectin;
COX, cyclooxygenase;
DAP, death-associated protein 1;
EZH, enhancer of zeste homolog;
Gprc, G-protein coupled receptor gene;
GSK, glycogen synthase kinase;
IL, interleukin;
LDLR, low-density lipoprotein receptor;
MCP, monocyte chemoattractant protein;
MCT, monocarboxylate transporter;
NEMO, NF-κB essential modulator;
NF-κB, nuclear factor-κB;
NKG, natural killer group;
PAP/HIP, pancreatitis-associated protein/hepatocarcinoma-intestine-pancreas;
PGC, peroxisome proliferator-activated receptor γ coactivator;
Rag, recombination activating gene;
SHP, Src homology region domain-containing phosphatase;
STAT, signal transducer and activator of transcription;
TNF, tumor necrosis factor;
TNFAIP, tumor necrosis factor, α-induced protein.

### Example 3: NK Cells Derived from Cox-2Flox/Flox;LysMCre/+ Mice Mediated Higher Cytotoxicity

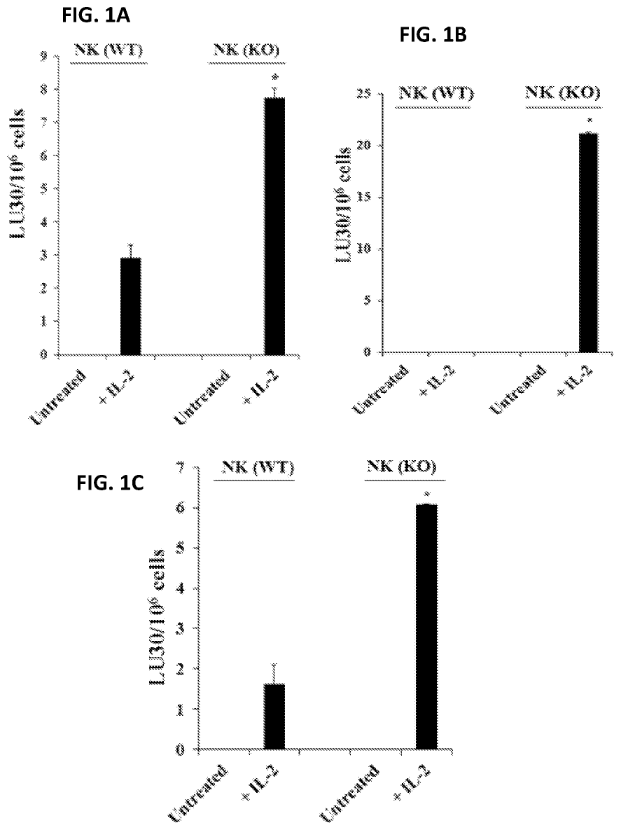

Purified NK cells obtained from spleens of control WT littermates (Cox-2flox/flox; LysM+/+) and those with targeted knockout of COX-2 gene in myeloid cells (Cox-2flox/flox; LysMCre/+) were left untreated or treated with IL-2 for 7 days before they were used in a standard ⁵¹Cr release assay against YAC-1 cells (FIG. 1A), Mouse Embryonic Fibroblasts (MEFs) (FIG. 1i), and ST63 cells (FIG. 1C). As shown in FIGS. 1A-1C, purified IL-2-treated NK cells from Cox-2flox/flox;LysMCre/+ mice lysed YAC-1 (P<0.05), MEFs (P<0.05), and ST63 cells (P<0.05) significantly more than IL-2-treated NK cells from control WT littermates which had no/low cytotoxicity. Untreated NK cells did not mediate any cytotoxicity (FIG. 1A-1C).

### Example 4: NK Cells Obtained from Cox-2Flox/Flox;LysMCre/+ Mice Cultured with Autologous Monocytes Mediated Significantly Higher Levels of Cytotoxicity than Those from Control Littermates Cultured with and without Monocytes

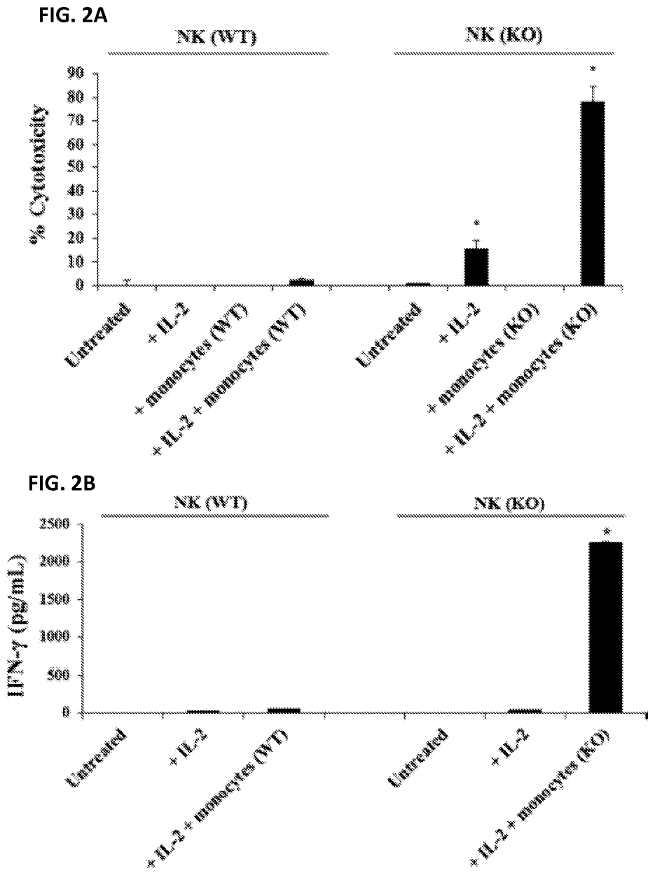

Purified NK cells from control WT littermates and Cox-2flox/flox;LysMCre/+ mice were cultured with or without purified autologous bone marrow derived monocytes for 7 days before the cells were used in a standard 4 h ⁵¹Cr release assay against YAC-1 tumors (FIG. 2A). As shown in FIG. 2A, IL-2-treated NK cells from control WT mice cultured with autologous monocytes mediated slightly higher cytotoxicity compared to the NK cells cultured without monocytes. IL-2-treated NK cells purified from Cox-2flox/flox;LysMCre/+ mice cultured with autologous monocytes lysed YAC-1 cells significantly more compared to NK cells cultured without autologous monocytes, and those purified from control WT animals cultured with autologous monocytes (P<0.05). IL-2-treated NK cells cultured with autologous monocytes from Cox-2flox/flox;LysMCre/+ mice also exhibited higher cytotoxicity against transformed mouse oral keratinocytes and MC38 cells (data not shown) as compared to NK cells from control WT littermates cultured with autologous monocytes.

### Example 5: NK Cells Purified from Cox-2Flox/Flox;LysMCre/+ Mice Cultured with Autologous Monocytes Produced Significantly Higher IFN-γ than Those from Control WT Littermates Cultured with and without Autologous Monocytes

Purified NK cells obtained from Cox-2flox/flox;LysMCre/+ mice and control WT littermates were cultured with or without purified autologous monocytes for 7 days, after which the supernatants were collected and the levels of IFN-γ produced by NK cells were measured with specific ELISA. Untreated NK cells did not secrete IFN-γ (FIG. 2B). IL-2 treated NK cells from both control WT and Cox-2flox/flox;LysMCre/+ mice produced much lower levels of IFN-γ in the absence of autologous monocytes (FIG. 2B). Significantly higher levels of IFN-γ were secreted by NK cells from Cox-2flox/flox;LysMCre/+ mice when cultured with autologous monocytes, whereas much lower amounts of IFN-γ could be seen in supernatants from NK cells from control WT littermates cultured with autologous monocytes (P<0.05) (FIG. 2B).

### Example 6: Step-Wise Increase in Cytotoxicity and IFN-γ Secretion when NK Cells from Control WT Mice or Cox-2Flox/Flox;LysMCre/+ Mice were Cultured with Wild Type or COX-2−/− Monocytes, Respectively

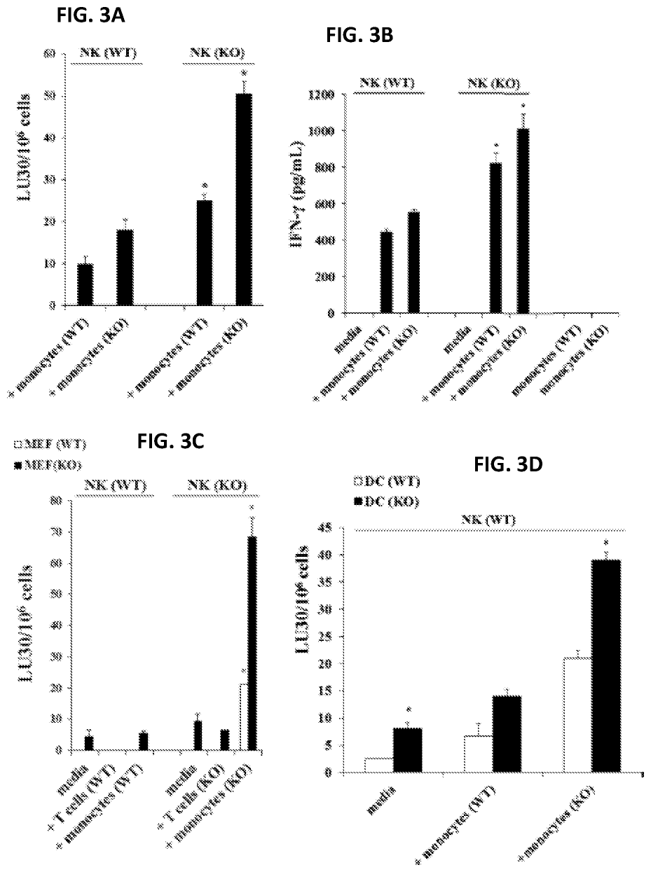

NK cells purified from either control WT littermates or Cox-2flox/flox;LysMCre/+ mice and treated with IL-2 were cocultured with either wild type monocytes or monocytes from Cox-2flox/flox;LysMCre/+ mice. The cytotoxic function of NK cells obtained from wild type mice against YAC-1 tumors remained at the lowest when cultured with wild type monocytes whereas an increase in the levels of cytotoxicity could be observed when they were cultured with monocytes from Cox-2flox/flox;LysMCre/+ mice (FIG. 3A). NK cells from Cox-2flox/flox; LysMCre/+ mice exhibited higher cytotoxicity either cultured with wild type monocytes (P<0.05) or COX-2 knockout monocytes (P<0.05) as compared to NK cells from wild type mice, although the levels were much higher when cultured with COX-2 knockout monocytes
(FIG. 3A). Therefore, the levels of IL-2-treated NK cell cytotoxicity from the lowest to highest in the co-cultures were as follows:
NK(WT)+MO(WT)<NK(WT)+MO(KO)<NK(KO)+MO(WT)<NK(KO)+MO(KO) (FIG. 3A). Secretion of IFN-γ in the co-cultures of NK cells with monocytes followed the same trend as seen with cytotoxicity. NK cells from wild type mice cultured with wild type monocytes
secreted the lowest amounts of IFN-γ when compared to those cultured with monocytes from Cox-2flox/flox;LysMCre/+ mice, which secreted the next highest levels (FIG. 3B). Significant amounts of IFN-γ were obtained when NK cells from Cox-2flox/flox;LysMCre/+ mice were cultured either with wild type (P<0.05) or Cox-2flox/flox;LysMCre/+ monocytes (P<0.05) (FIG. 3B). IL-2-treated NK cells in the absence of monocytes from both wild type and Cox-2flox/flox;LysMCre/+ mice did not secrete detectable IFN-γ (FIG. 3B). Similarly, monocytes in the absence of NK cells did not secrete IFN-γ (FIG. 3B).

### Example 7: COX-2 Gene Deletion in Mouse Embryonic Fibroblasts (MEFs) Resulted in a Significant Susceptibility to NK Cell-Mediated Lysis

Purified NK cells obtained from spleens of control WT littermates and Cox-2flox/flox; LysMCre/+ mice were cultured with or without monocytes in the presence of IL-2 treatment. Afterward, the NK cells were used as effectors in a standard ⁵¹Cr release assay against wild type and COX-2−/− MEFs from global COX-2−/− mice. As shown in FIG. 3C, NK cells from wild type mice cultured with and without autologous monocytes mediated lower levels of cytotoxicity against COX-2−/− MEFs when compared to NK cells from Cox-2flox/flox;LysMCre/+ mice cultured with and without autologous monocytes (P<0.05), and lower cytotoxicity could be observed against wild type MEFs (P<0.05). NK cells obtained from Cox-2flox/flox;LysMCre/+ mice cultured with autologous monocytes had the greatest cytotoxicity against both wild type (P<0.05) and COX-2−/− MEFs (P<0.05), although the highest levels were seen against COX-2−/− MEFs when compared to wild type MEFs (P<0.05) (FIG. 3C).

### Example 8: Co-Culture with COX-2−/− Monocytes, but not COX-2−/− T Cells, Increased the Cytotoxic Function of NK Cells

NK cells and monocytes were purified from either control WT littermates or Cox-2flox/flox;LysMCre/+ mice. T cells were purified from wild type or global COX-2 knockout mice. NK cells were treated with IL-2 and cultured alone or with purified CD3+ naïve T cells or monocytes from wild type or COX-2−/− mice. Afterward, T cells and monocytes were removed from the co-cultures and NK cells were used as effector cells against wild type and COX-2−/− MEFs in a standard ⁵¹Cr release assay (FIG. 3C). The cytotoxic function of NK cells from wild type mice was lower against both wild type and COX-2−/− MEFs, and the addition of either T cells or monocytes from wild type mice did not increase the cytotoxicity significantly (FIG. 3C). NK cells obtained from Cox-2flox/flox;LysMCre/+ mice cultured with autologous monocytes, but not with T cells from global COX-2−/− mice, increased cytotoxicity of NK cells significantly against both wild type (P<0.05) and COX-2−/− MEFs (P<0.05). Albeit, the highest increase could be observed against COX-2−/− MEFs when compared to wild type MEFs, while the addition of T cells from global COX-2 knockout mice did not have significant effect on NK cell cytotoxicity (FIG. 3C).

### Example 9: Dendritic Cells Derived from Monocytes of Cox-2Flox/Flox;LysMCre/+ Mice were More Susceptible to NK Cell-Mediated Cytotoxicity than Dendritic Cells from Wild Type Mice

Dendritic cells were derived from purified monocytes by the addition of IL-4 and GM-CSF for 7 days. Differentiated DCs from wild type or Cox-2flox/flox;LysMCre/+ mice were labeled with ⁵¹Cr and used as targets in a standard ⁵¹Cr release assay against IL-2-treated NK cells derived from wild type mice in the presence and absence of monocytes. As predicted, DCs differentiated from Cox-2flox/flox;LysMCre/+ monocytes were more susceptible to IL-2-treated NK cell-mediated lysis as compared to those differentiated from monocytes obtained from control WT littermates (P<0.05) (FIG. 3D). NK cells purified from control WT mice cultured with monocytes from Cox-2flox/flox;LysMCre/+ mice induced the highest lysis of Cox-2flox/flox;LysMCre/+ DCs when compared to DCs from wild type mice (P<0.05) (FIG. 3D). Although NK cells obtained from wild type mice cultured with autologous monocytes lysed Cox-2flox/flox;LysMCre/+ DCs more as compared to wild type DCs, the levels of cytotoxicity were significantly lower when compared to NK cells obtained from wild type mice and cultured with monocytes from Cox-2flox/flox;LysMCre/+ mice (P<0.05) (FIG. 3D).

### Example 10: LPS Induced Split Anergy in Murine NK Cells as Evident by a Decrease in Cytotoxicity and an Increase in IFN-γ Secretion by NK Cells

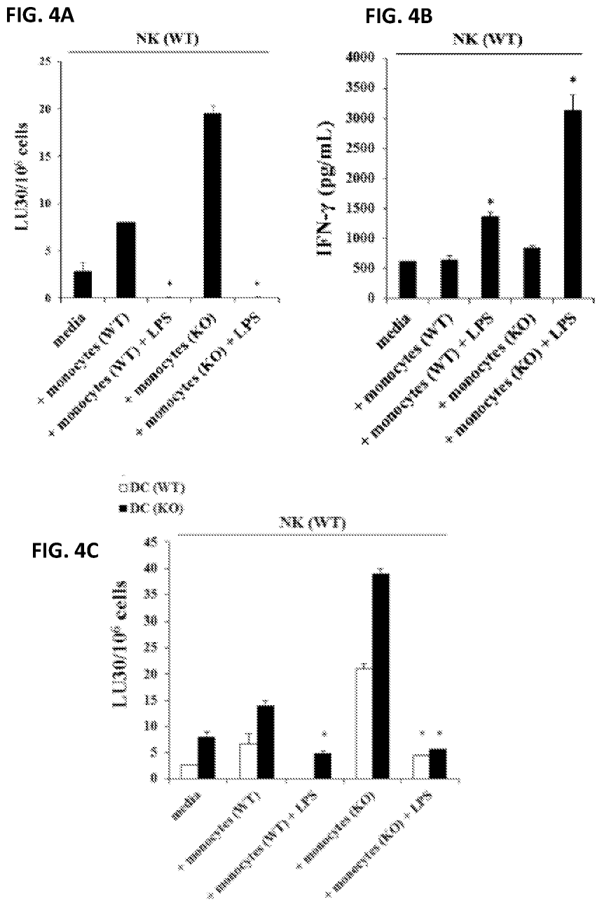

Purified NK cells obtained from control WT littermates were treated with IL-2 and cultured in the presence of autologous monocytes or those obtained from Cox-2flox/flox; LysMCre/+ mice in the absence and presence of LPS before they were used in a standard ⁵¹Cr release assay against YAC-1 tumors. The addition of LPS to the cultures of IL-2-treated NK cells with monocytes from control WT littermates resulted in a complete shutdown of the NK cells' ability to lyse YAC-1 cells (P<0.05) (FIG. 4A) while it increased the amount of IFN-γ secreted by the NK cells (P<0.05) (FIG. 4B). Similarly, IL-2-treated NK cells obtained from control WT littermates cultured with monocytes from Cox-2flox/flox;LysMCre/+ mice significantly increased NK cell-mediated cytotoxicity against YAC-1 cells when compared to control WT littermate NK cells cultured with autologous monocytes, and the addition of LPS completely abolished cytotoxicity (P<0.05) (FIG. 4A) while increasing IFN-γ secretion significantly (P<0.05) (FIG. 4B). Similar results to those seen with YAC-1 targets were also seen when control and Cox-2flox/flox;LysMCre/+ DCs were used as targets (FIG. 4C). As shown in FIG. 4C, the addition of LPS to IL-2-treated NK cells from wild type mice either cultured with autologous monocytes or with monocytes obtained from Cox-2flox/flox;LysMCre/+ mice resulted in decreased NK cell cytotoxicity against both wild type (P<0.05) and Cox-2flox/flox;LysMCre/+ DCs (P<0.05), albeit the decrease was substantially more with Cox-2flox/flox;LysMCre/+ DCs when compared to wild type DCs (FIG. 4C). When NK cells from C57bl6 mice unrelated to the breeding colony control littermates for COX-2 were used in the presence and absence of autologous monocytes with and without LPS, similar results to those obtained with NK cells from control WT littermates in regards to cytotoxicity and IFN-γ secretion were seen.

### Example 11: Decreased Constitutive Expression of MHC Class-I on COX-2−/− MEFs and Increased Expression after Treatment with IFN-γ and/or TNF-α

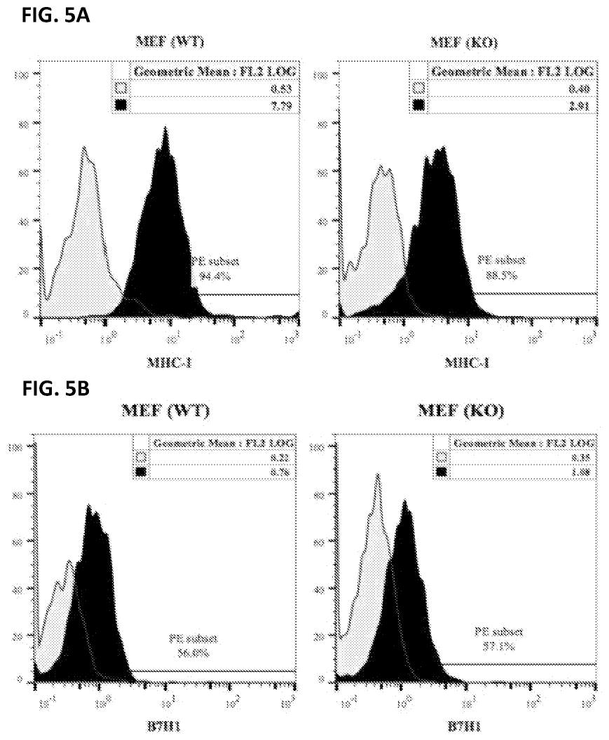

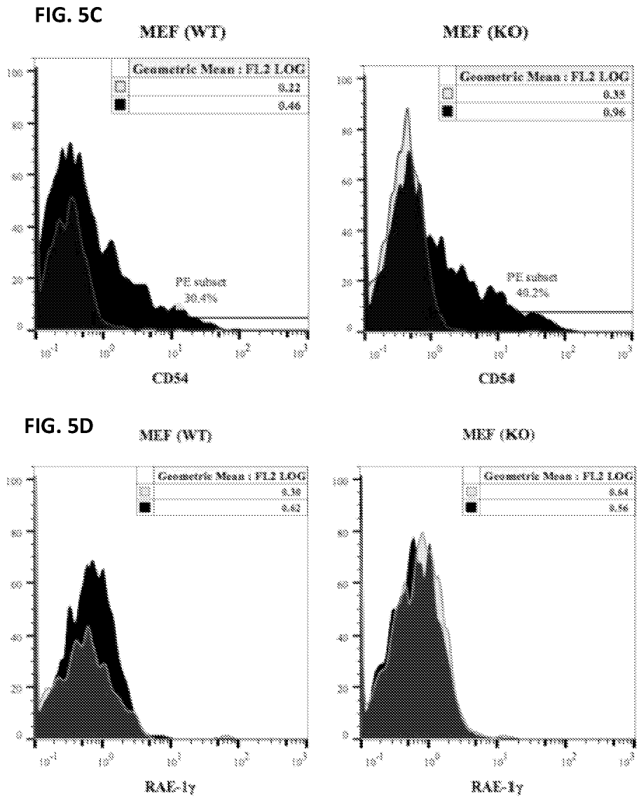

Expression of MHC class-I (FIG. 5A), B7H1 (FIG. 5B) and CD54 (FIG. 5C) were determined on wild type and COX-2−/− MEFs. COX-2−/− MEFs demonstrated lower expression of MHC class I but no significant change for B7H1 or CD54 expression for untreated MEFs (FIG. 5A-FIG. 5C), whereas those treated with IFN-γ expressed higher MHC class-I, B7H1 and CD54 surface receptors when compared to wild type MEFs. The addition of TNF-α to COX-2−/− MEFs increased MHC class-I and CD54 but had no effect on B7H1 expression when compared to wild type MEFs. Treatment with the combination of IFN-γ and TNF-α synergistically increased MHC class-I, B7H1, and CD54 on both wild type and COX-2−/− MEFs, however, the levels of expression were higher on COX-2−/− MEFs when compared to wild type MEFs. Decrease in constitutive expression of MHC class-I on untreated COX-2−/− MEFs were seen for the majority of the experiments, however, its modulation with IFN-γ and/or TNF-α were variable, demonstrating an increase on COX-2−/− MEFs as compared to wild type MEFs in most experiments; but in a few experiments a decrease rather than an increase was noted on COX-2−/− MEFs as compared to wild type MEFs which depended on cell passage number and growth dynamics (data not shown). No expression of Rae-17 could be seen on either wild type or COX-2−/− MEFs (FIG. 5D).

### Example 12: Significant Down-Modulation of NK Cell Receptors after their Culture with MEFs and Monocytes

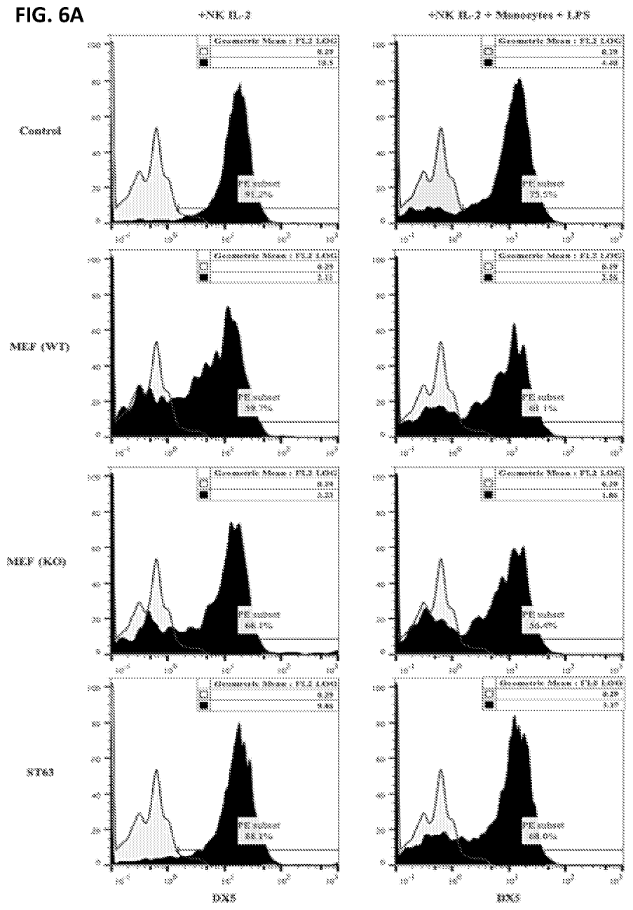

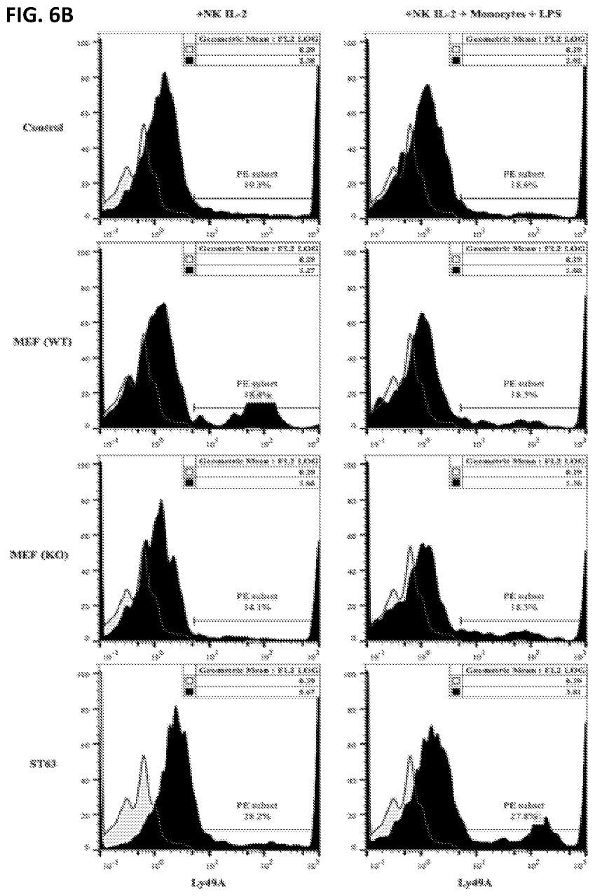

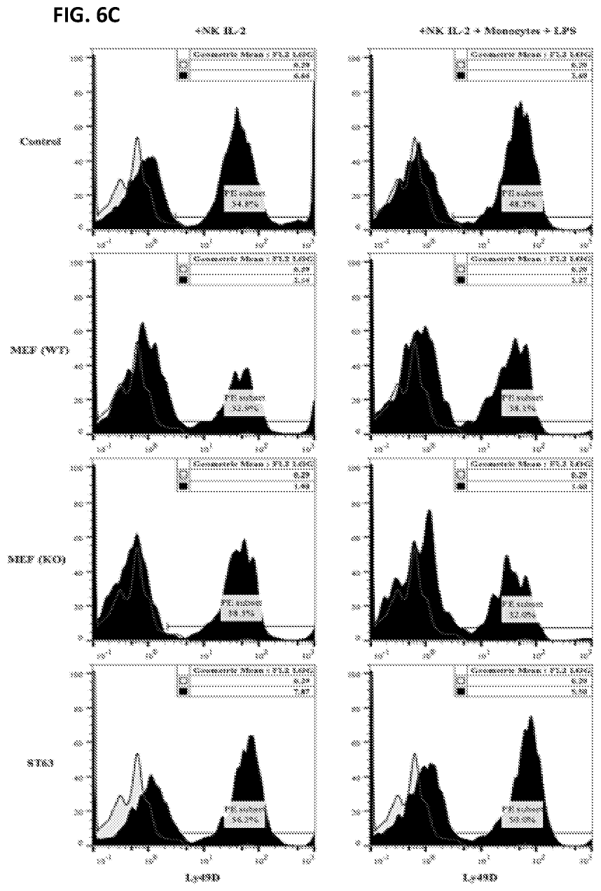

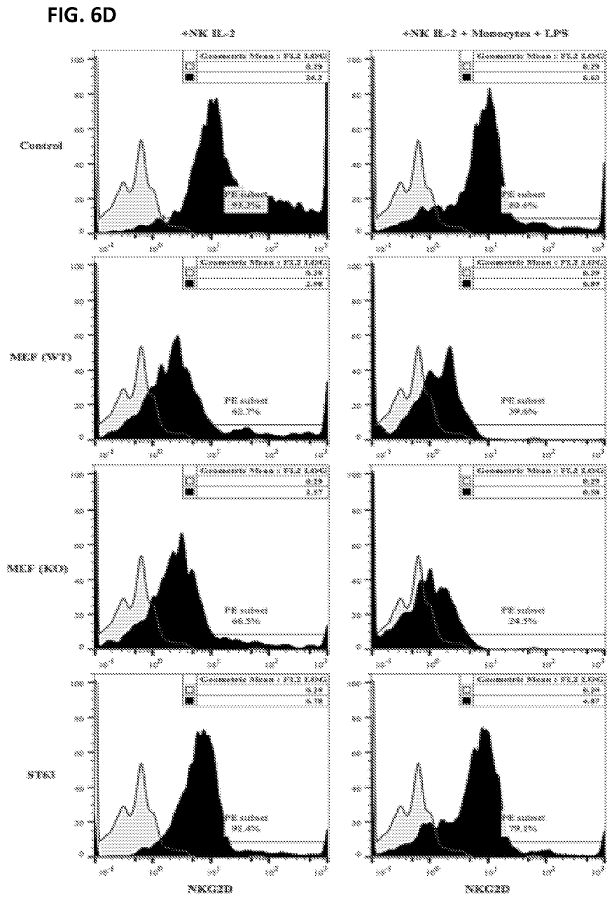

The expression of DX5 (FIG. 6A), Ly49A (FIG. 6B), Ly49D (FIG. 6C), and NKG2D (FIG. 6D) were determined on the surface of NK cells activated with IL-2 and cultured with and without monocytes and LPS in the presence and absence of wild type and COX-2−/− MEFs or ST63. A generalized decrease in all four receptor expression on NK cells were noted after culture with wild type or COX-2−/− MEFs (FIG. 6), whereas the expression of DX5 and NKG2D was either decreased or not changed on NK cells after interaction with ST63. In contrast, an increase in the expression of Ly49A and Ly49D was seen on NK cells cultured with ST63 cells (FIG. 6). Culture of NK cells with monocytes also exhibited significant down-modulation of all four receptors in the absence and presence of culture with wild type and COX-2−/− MEFs and ST63 cells (FIG. 6).

### Example 13: Monocytes in the Presence and Absence of LPS Induced Split Anergy in IL-2 Treated Human NK Cells

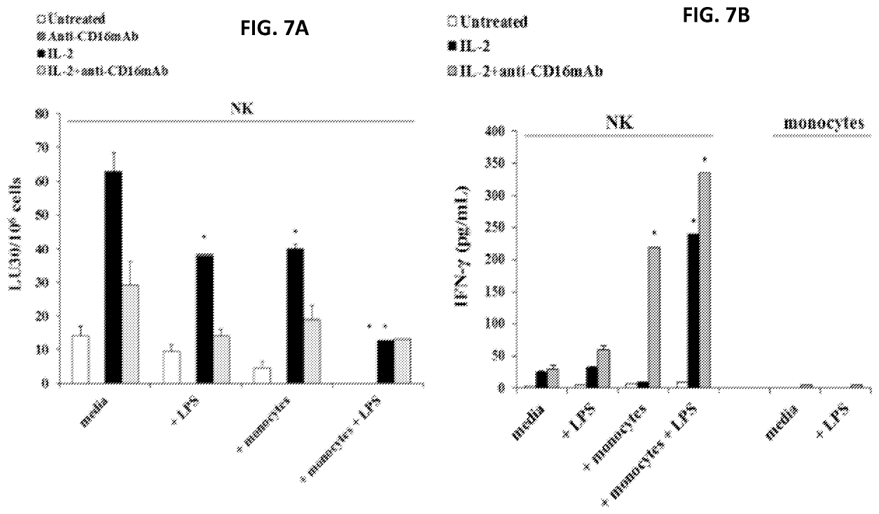

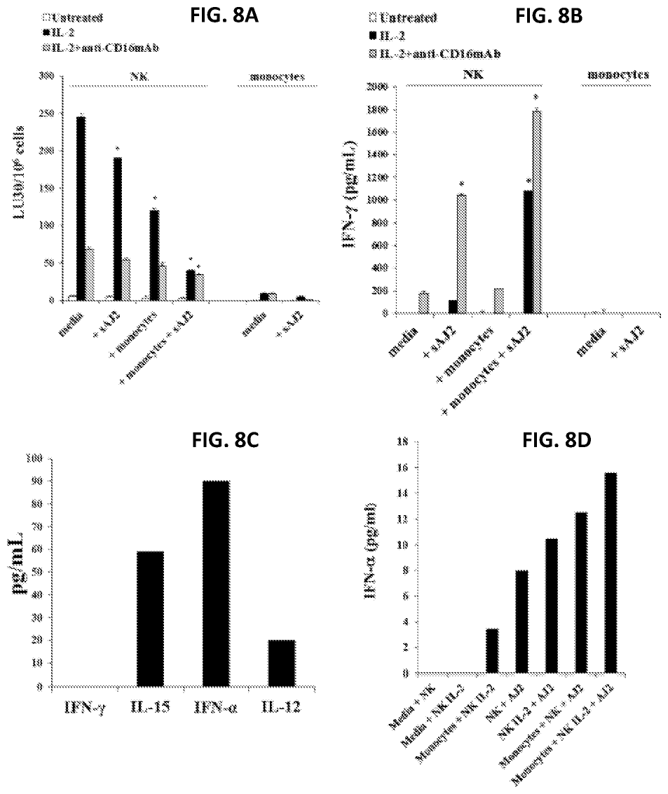

The addition of LPS, as well as sAJ2, to human NK cells in the absence and presence of monocytes resulted in the significant induction of split anergy (FIG. 7 and FIG. 8). As demonstrated in FIG. 7, LPS induced loss of NK cell-mediated cytotoxicity against Oral Squamous Carcinoma Stem Cells (OSCSCs) while increasing IFN-γ secretion. Unlike mouse NK cells in which culture of monocytes with IL-2-treated NK cells increased NK cell cytotoxicity and secretion of IFN-γ, culture of monocytes with IL-2-treated NK cells from humans inhibited cytotoxicity (P<0.05) while increasing IFN-γ secretion (P<0.05) (FIG. 7). The highest decrease in cytotoxicity and increase in IFN-γ secretion were observed when IL-2 or IL-2 and anti-CD16mAb-stimulated NK cells cultured with monocytes were treated with LPS (P<0.05) (FIG. 7). Split anergy in human NK cells was also induced by gram-positive bacteria sAJ2 (FIG. 8). The loss of cytotoxicity in IL-2-treated NK cells was induced with the addition of monocytes in the presence or absence of sAJ2 while it induced significant secretion of IFN-γ (P<0.05) (FIG. 8A, FIG. B). The highest decrease in cytotoxicity and increase in IFN-γ secretion was obtained when IL-2 or IL-2 and anti-CD16mAb-treated NK cells were cultured with monocytes and treated with sAJ2 (P<0.05) (FIG. 8A, FIG. B). In addition to IFN-γ, the levels of IL-6, IL-8, IL-10, GM-CSF, and TNF-α were also increased when NK cells were cultured with monocytes and bacteria. No release of MICA or MICB could be seen in the cultures of NK cells with monocytes (data not shown), even though the same treatment induces significant IL-6 and IL-8 release in the co-cultures of NK cells with monocytes. Therefore, although monocytes increased IFN-γ secretion in both species, they inhibited cytotoxicity by human NK cells whereas they increased cytotoxicity by mouse IL-2-treated NK cells. Treatment of NK cells and monocytes with LPS, on the other hand, inhibited cytotoxicity in both human and mouse NK cells while increasing IFN-γ secretion substantially. Human monocytes secreted significant levels of NK activating cytokines IL-15, IFN-α, and IL-12 (FIG. 8C) and the levels of IFN-α increased when cultured with the NK cells (FIG. 8D).

### Example 14: Decreased Total Cell Counts but within Total Cells Increased Percentages of NK Cells and Monocytes in the BM and Spleen of Rag2^{-/-} Mice in Comparison to WT Mice

Rag1^{-/-} and Rag2^{-/-} mice fail to produce mature B or T lymphocytes due to total inability to initiate V(D)J rearrangements but are able to produce Natural Killer (NK) cells with immune intact controls. Murine and human NK cells do not require the receptor gene rearrangement for the development, in fact NK cells in Rag2^{-/-} mice express more mature and activated phenotype at resting state compared to NK cells from WT mice, Rag2^{-/-} NK cells express KLRG1^{high}CD27^{low}CD11b^{high}CD69^{high}CD62^{low}. In general NK cells constitute approximately 3-5% of total lymphocytes in peripheral blood and secondary lymphoid organs, but NK cells proportions are approximately 15-30% of total lymphocytes in Rag2-mice and these NK cells per cell basis are more cytotoxic both in-vitro and in-vivo compared to WT mice. Major role of NK cells includes direct NK cell-mediated cytotoxicity, antibody-dependent cellular cytotoxicity (ADCC) and indirect regulation of other immune cells and tumor differentiation through inflammatory cytokine and chemokine secretions.

NK cells express both RANKL and M-CSF and can trigger monocytes' differentiation into osteoclasts, a process dependent on RANKL and M-CSF. Although the role of NK cells in osteoblast regulation and bone remodeling is not well documented but IFN-γ produced by both NK cells and Th1 lymphocytes, has been shown to inhibit osteoclastogenesis in vitro. NK cells also synthesize TNF-α which can increase RANKL expression and RANKL dependent osteoclastogenesis. Bisphosphonates (BPs) are drugs those are used for a variety of bone diseases exhibiting excessive osteoclastic activity including Paget's disease of the bone, metastatic and osteolytic bone disease, hypercalcemia of malignancy and osteoporosis, BPs play very important role in regulating the osteoclast-mediated bone resorptive activity by various ways including osteoclast recruitment, differentiation and apoptosis. It has been previously demonstrated that BPs were able to increase cytokine and chemokine secretion by the NK cells when co-cultured with osteoclast.

Osteonecrosis of the jaw (ONJ), also known as bisphosphonate-related osteonecrosis of the jaw (BRONJ), BRONJ is usually clinically diagnosed and is defined as an area of exposed bone in the maxillofacial region that does not heal within 8 weeks after identification by health care provider. The prolonged jaw pain, tooth mobility, bone enlargement, gingival swelling, erythema, and ulceration are among major signs and symptoms that may occur before the development of clinically detectable osteonecrosis, and can become symptomatic when the surrounding soft tissues become inflamed. The first case of ONJ or BRONJ was reported in 2003, with the intake of pamidronate (Aredia) and zolendronate (Zometa) (ZOL) demonstrating osteonecrosis of the jaw in patients receiving bisphosphonates. Majority (95%) of BRONJ reported cases are among cancer patients receiving high-dose intravenous BPs and approximately 5% of the reported cases are among osteoporosis patients receiving low-dose BPs. On average 1-10 of 100 cancer patients on high dose of BPs develop ONJ. The potency of the bisphosphonate, frequency of administration, and duration of use influence ONJ risk; IV BPs induce more ONJ compared to orally taken BPs. Although ONJ can appear in both jaw but mandibular cases have reported approximately twice as maxillary.

To define the function of NK cells and severity of BRONJ in Rag2^{-/-} mice, various tissue compartments were analyzed herein and compared those with WT mice. It is discovered herein that processed tissues from Rag2^{-/-} mice have low number of immune cells, and in those immune cells there are increased percentages of NK cells and monocytes in the presence of very low T cells compared to WT mice. It is further discovered herein that immune cells from various tissues in Rag2^{-/-} mice demonstrated increases secretion of IFN-γ whereas NK cell-mediated cytotoxicity was very similar with the exception of spleen and spleen-purified NK cells which was higher in Rag2^{-/-} mice. Last, when ZOL was injected to Rag2^{-/-} mice, there was increased secretion of IFN-γ and NK cell-mediated cytotoxicity in bone marrow, reverse effect was seen in spleen, spleen-purified NK cells and oral gingiva. Interestedly no ONJ was seen in Rag2^{-/-} mice and less ZOL-induced activation of immune cells was seen in comparison to ZOL-induced immune cells activation in WT mice.

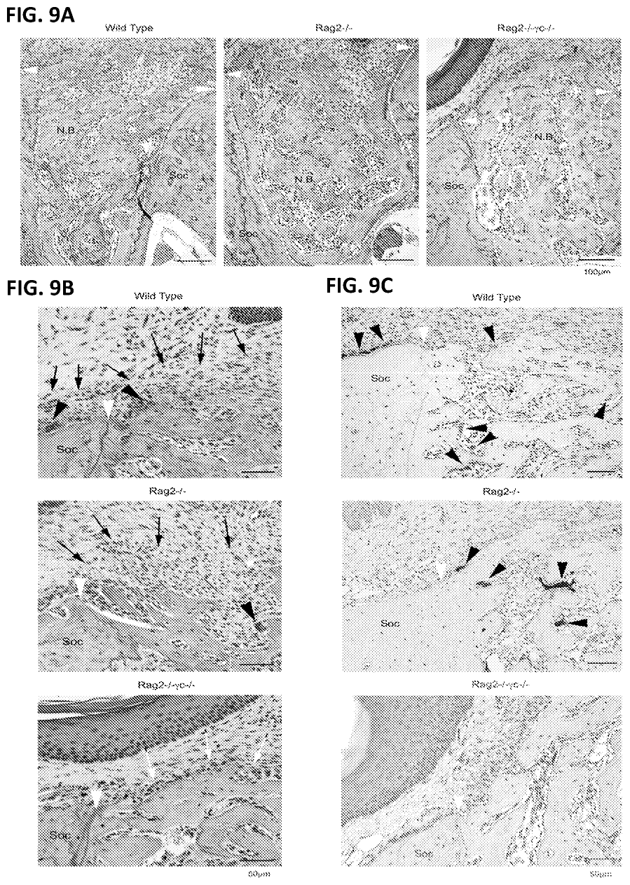

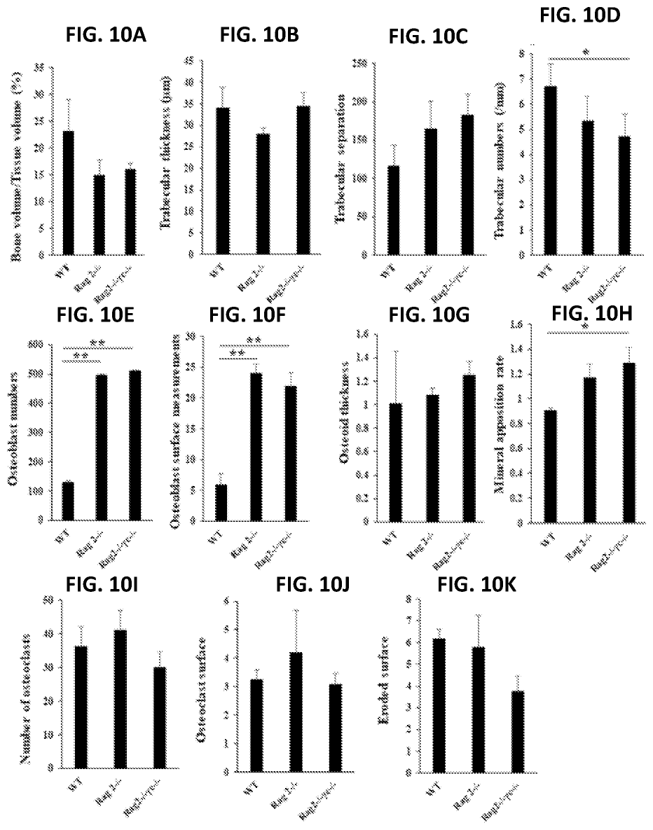

To evaluate the phenotypic differences in WT and Rag2^{-/-} mice, it was determined herein the total number of immune cell counts and percentages of immune cells subsets in the lymphocytes in the bone marrow and spleen of both mice models. As shown in FIG. 9 and FIG. 10, there were significantly lower cell counts in the bone marrow (FIG. 9A) and the spleen (FIG. 10A) of Rag2^{-/-} mice in comparison to WT mice (FIGS. 9A and 10A). When the percentages of Nkp46+ NK cells, DX5+ NK cells, F4/80+ monocytes were determined in the CD45+ immune cells of the BM and spleen, the percentages of these immune subsets were significant higher both in BM (FIG. 9B) and spleen (FIG. 10B) of the Rag2^{-/-} mice in comparison to WT mice (FIGS. 9B and 10B). In addition, the percentages of CD3+ T cells were significantly lower in both tissues of the Rag2^{-/-} mice in comparison to WT mice (FIGS. 9B and 10B). Next, when the NK cells were sorted out from the total splenocytes of both mice models, number of purified NK cells isolated in case of Rag2^{-/-} mice were significantly higher in comparison to WT mice (FIG. 10C).

### Example 15: Increased Percentages of NK Cells in the Oral Gingival Tissue of Rag2^{-/-} Mice in Comparison to WT Mice

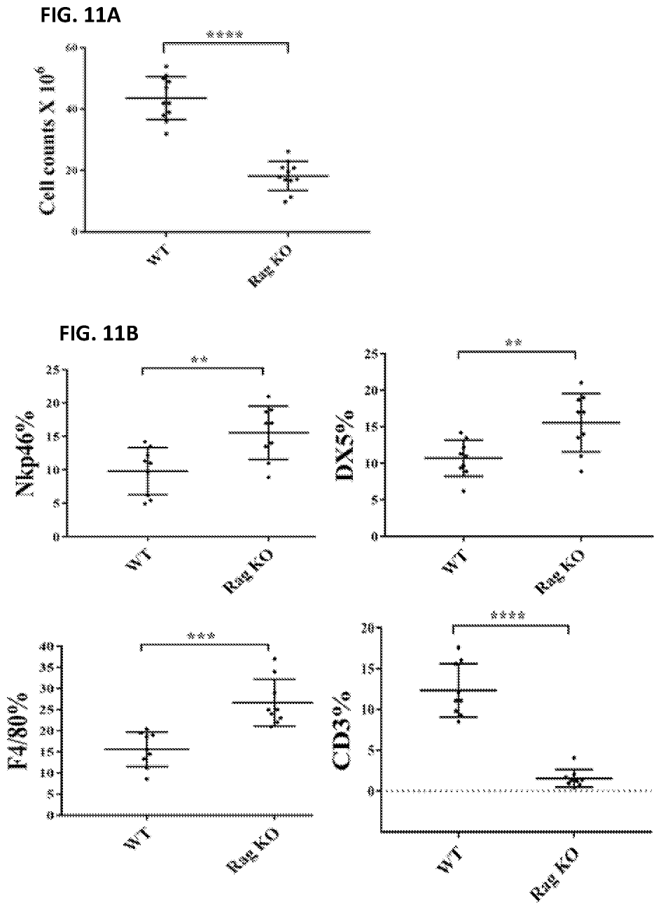

The oral gingival tissue was then dissociated to recover single cell population and this single cell population of gingival cells was used to determine the proportion of DX5+NK cells. As shown in FIG. 3, the number of DX5+NK cells in gingival cells of Rag2^{-/-} mice was significantly higher in comparison to WT mice (FIGS. 11A and 11B).

### Example 16: Increased IFN-γ Secretion and NK Cell-Mediated Cytotoxicity by Various Tissue Compartments in Rag2^{-/-} Mice in Comparison to WT Mice when Adjusted on Cell Basis

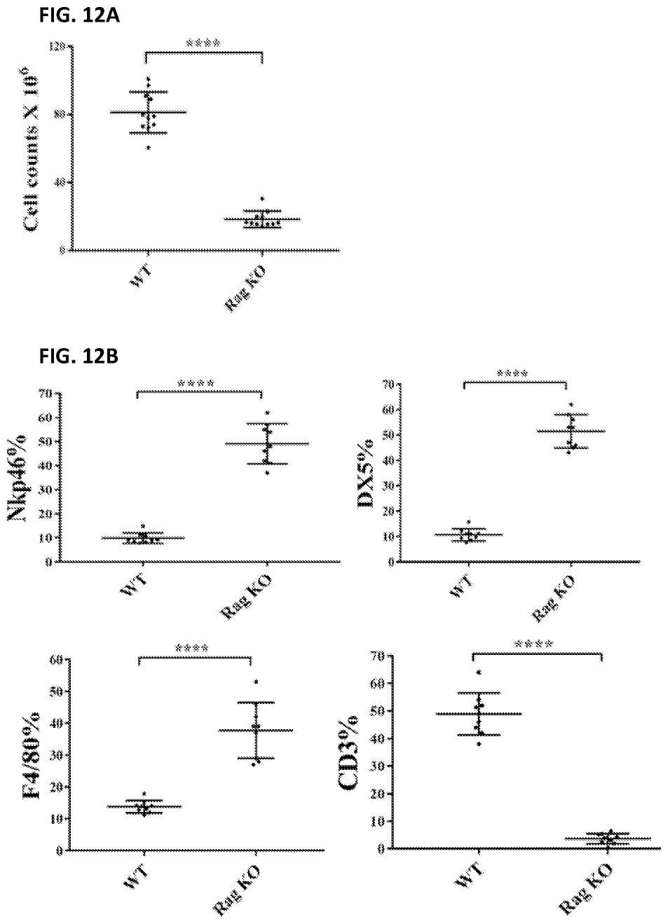

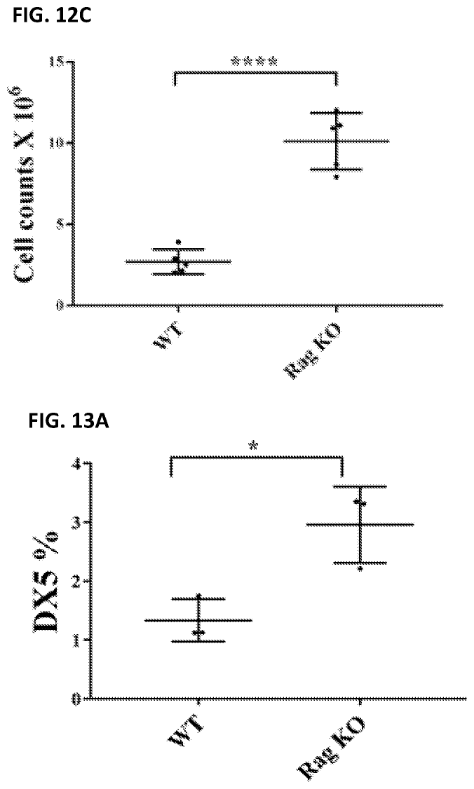

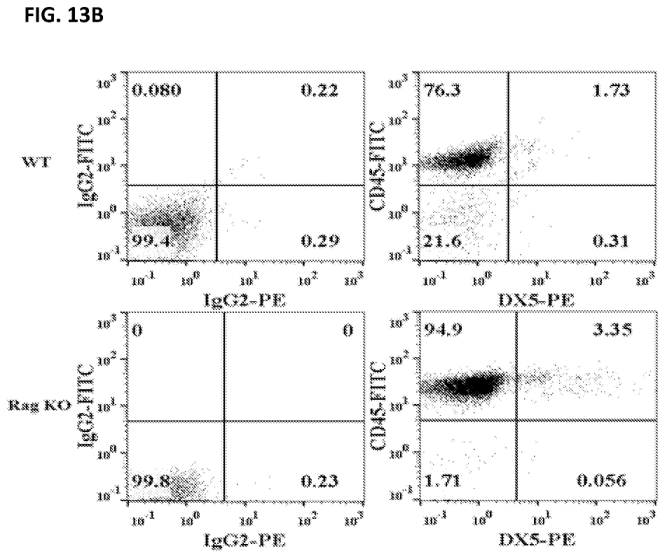

When the function of immune cells were assessed in various tissue compartment of both mice models, both IFN-γ secretion and NK cell-mediated cytotoxicity were higher in Rag2^{-/-} mice in comparison to WT mice (FIGS. 12 and 13). A similar number of cells were cultured for the same time points. Immune cells from BM, spleen, pancreas, peri-pancreatic adipose tissue, and spleen-purified NK cells were analyzed. IFN-γ secretion from these tissues was higher in Rag2^{-/-} mice in comparison to WT mice (FIG. 12), not much difference was seen in the IFN-γ secretion of oral gingiva cells (FIG. 12). In assessments for NK cell-mediated cytotoxicity, BM showed slight to moderate increase—not much difference was seen in pancreas and per-pancreatic adipose tissue, but spleen and spleen-purified NK cells exhibit higher NK cell-mediated cytotoxic function in higher in Rag2^{-/-} mice in comparison to WT mice (FIG. 12).

### Example 17: ZOL Induced Increased IFN-γ Secretion and NK Cell-Mediated Cytotoxicity by BM Cells but Reverse Effect was Seen in Splenocytes, NK Cells and Oral Gingiva Cells in Rag2^{-/-} Mice

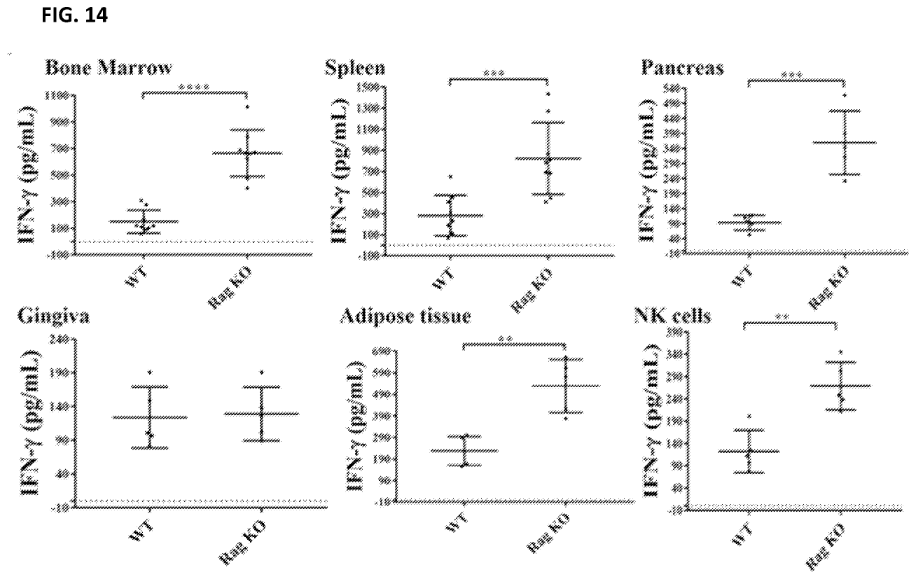

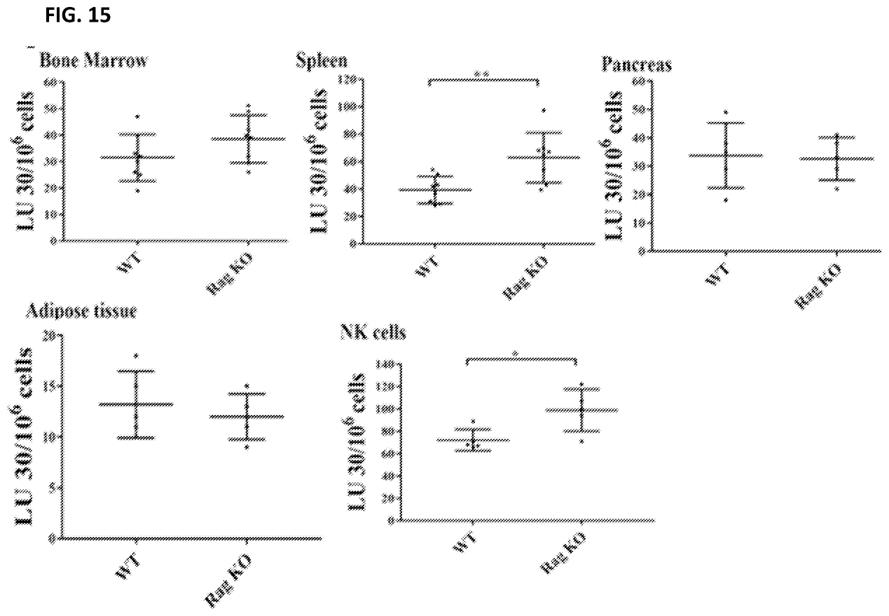

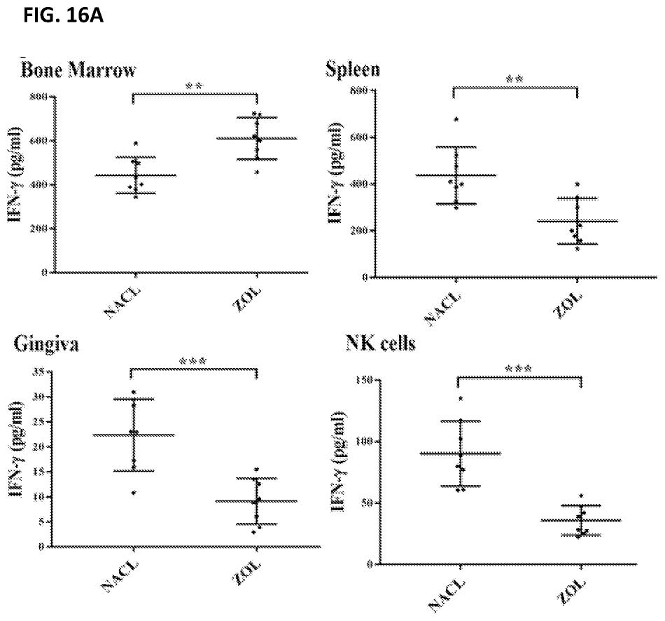

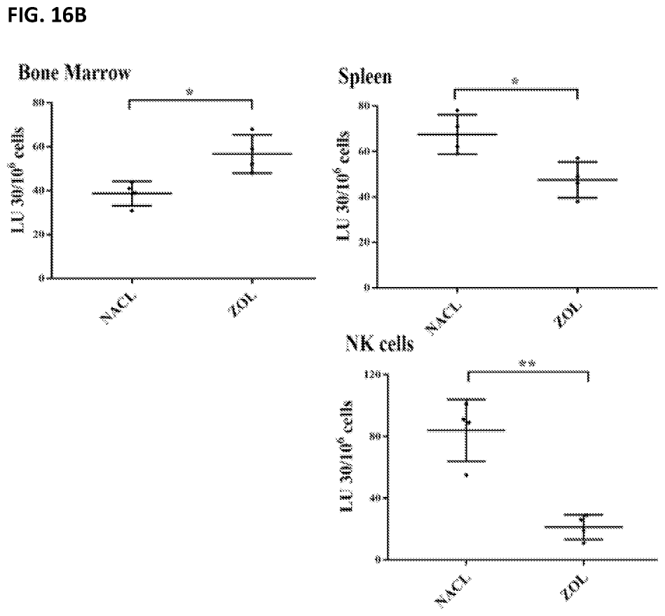

To evaluate the effect of ZOL in Rag2^{-/-} mice, IFN-γ secretion and NK cell-mediated cytotoxic function induced by BM, splenocytes, spleen-purified NK cells and oral gingiva of the Rag2^{-/-} mice after they were administered with ZOL followed by maxillary left first molar extraction was determined. Mice administered NaCl were used as controls. ZOL induced immune cells activation in BM, both IFN-γ (FIG. 14) and NK cell-mediated cytotoxicity were significantly increased after ZOL injections (FIG. 14). Interestingly, a reverse effect was seen in other tested tissue compartments. In spleen, spleen-purified NK cells, and oral gingiva cells ZOL intake blocked the secretion of IFN-γ (FIG. 14), similarly NK cell-mediated cytotoxicity was blocked with ZOL in spleen and spleen-purified NK cells (FIG. 14).

### Example 18: ZOL Induced Effect Levels were Higher in WT Mice in Comparison to Rag2^{-/-} Mice

When ZOL was injected to both WT and Rag2^{-/-} mice, different levels of ZOL-induced activation were revealed in various immune tissue compartments of the both mice models. After ZOL injection, there were 4-6 fold (300-500%) increases in IFN-γ of BM from the WT mice, whereas this ZOL-induced increased IFN-γ was only 1.5-1.75 fold (50-75%) in Rag2^{-/-} mice BM (Table 2). In spleen ZOL induced 2-3.4 fold (100-240%) increase in the IFN-γ of splenocytes from the WT mic, but ZOL-induced decreased IFN-γ in Rag2^{-/-} mice splenocytes, drop was 0.5-0.66 fold (34-50% drop) (Table 2). Very similar to splenocytes, NK cells purified from splenocytes showed increased secretion of IFN-γ by 1.2-1.9 fold (20-90% increase) in the presence of ZOL injections, opposite to this IFN-γ was decreased by 0.35-0.42 fold (58-65% drop) in Rag2^{-/-} mice NK cells in the presence of ZOL injections (Table 2). IFN-γ secretion was also differently modulated in the oral tooth extraction site in both mice models, in WT mice ZOL resulted in 0.3-0.5 fold change (50-70% decrease), and in Rag2^{-/-} mice ZOL resulted in 0.22-0.35 fold change (65-78% decrease) (Table 2).

TABLE 2
ZOL induced effect levels were higher in WT mice
in comparison to Rag2^{-/-} mice
Fold change in IFN-y secretion (ZOL group/NaCl group)

| | |
|---|---|
| WT mice | Rag KO mice |

Bone Marrow

| | | |
|---|---|---|
| IFN-γ | 4-6 | 1.5-1.75 |

Spleen

| | | |
|---|---|---|
| IFN-γ | 2-3.4 | 0.5-0.66 |

Spleen-purified NK cells

| | | |
|---|---|---|
| IFN-γ | 1.2-1.9 | 0.35-0.42 |

Gingiva

| | | |
|---|---|---|
| IFN-γ | 0.3-0.5 | 0.22-0.35 |

WT and RAG2 mice were administered with either 0.9% NaCl or ZOL (500 μg/kg) via tail vein followed by maxillary left first molar extraction as described in Materials and Methods section. Week 4 after the tooth extraction, animals were sacrificed, BM cells, splenocytes, splenocytes-purified NK cells and oral gingiva cells were cultured at (1×10⁶ cells/ml) each treated with IL-2 (10000 U/ml) for 5 days, after which the supernatants were harvested and the levels of IFN-γ were determined using specific ELISA. Modulation of IFN-7 by ZOL in both WT and RAG2 mice models were determined, and fold change in comparison to NaCl controls was calculated for each tissue.

### Example 19: NK Cell Survival, Cytotoxicity, Proliferation and Cytokine Secretion were Significantly Increased when Co-Cultured in the Presence of HEp2-IκB(S32AS36A) Cells

Decreased survival and function of NK cells in the presence of vector-alone transfected HEp2 cells is in stark contrast to increased expansion and function of NK cells co-cultured in the presence of HEp2-IκB(S32AS36A) cells (Table 3).

TABLE 3
Increased NK cell survival, cytotoxicity, proliferation and cytokine
secretion when co-cultured in the presence of HEp2-IκB(S32AS36A) cells

| Experiment | | | pRcCMV | | pRcCMV/IκB( [symbol] ) | |
|---|---|---|---|---|---|---|
| # cell survival | IL [symbol] | no targets | − IFN-γ | + IFN-γ | − IFN-γ | + IFN-γ |
| | − | 4.1 × 10 [symbol] | 2.8 × 10 [symbol] | 6.0 × 10 [symbol] | 1.12 × 10 [symbol] | 7.2 × 10 [symbol] |
| | + | 1.21 × 10 [symbol] | 2.0 × 10 [symbol] | 1.0 × 10 [symbol] | 4.32 × 10 [symbol] | 3.24 × 10 [symbol] |
| Proliferation | − | 4053 ± 41 | 7243 ± 164 | 4227.5 ± 916 | 6226 ± 100 | 6444 ± 254 |
| | + | 30494 ± 1000 | 18557 ± 933 | 6444 ± 127 | 52353 ± 4998 | 47116 ± 5528 |
| IFN-γ secretion | − | 0.205 ± 0.115 | 0.23 ± 0.27 | 0.075 ± 0.035 | 29.65 ± 4.19 | 20.7 ± 2.92 |
| | + | 64.88 ± 4.15 | 37.2 ± 0.31 | 11.5 ± 0.212 | 95.6 ± 5.2 | 77.37 ± 9.75 |
| % cytotoxicity | − | — | 20 ± 3 | 4 ± 1.8 | 34 ± 1 | 16 ± 2 |
| | + | — | 31 ± 0.06 | 8 ± 3 | 46 ± 3 | 20 ± 2.8 |

[symbol]

indicates data missing or illegible when filed

NK cells were treated with and without IL-2 (500 units (u)/ml) overnight before their co-culture in the presence and absence of IFN-γ (500 u/ml) treated pRcCMV vector-alone and HEp2-IκB(S32AS36A) transfected HEp2 cells. HEp2 cell treatments with IFN-γ were carried out in an overnight assay. ⁵¹Cr release assay was conducted after 12-18 hours of incubation, IFN-γ ELISA after 24-48 hours, 3H thymidine incorporation assay and the determination of the number of NK cell survival after 5 days of NK cell co-culture with HEp2 tumor cell transfectants. The p value for the differences between NK cell function and survival between vector-alone transfected HEp2 cells and IκB(S32AS36A) transfected HEp2 cells are less than 0.05 for all the samples tested.

### Example 20: Functional Inactivation and Depletion of CD8+ T Cells after Interaction with Vector-Alone Transfected HEp2 Cells but not with HEp2-IκB(S32AS36A) Cells

Untreated and IL-2 treated purified CD8+ T cells were co-cultured in the absence and presence of vector-alone transfected HEp2 cells and HEp2-IκB(S32AS36A) cells. The levels of CD8+ T cell survival were assessed by microscopy as well as by flow cytometric analysis of live T cells, and the rate of T cell proliferation by measuring the amounts of ³H thymidine incorporation in T cells before and after interaction with tumor cell transfectants. The addition of vector-alone transfected HEp2 cells to CD8+ T cells resulted in a significant depletion of T cell numbers in the co-cultures of T cell and vector-alone transfected HEp2 cells when compared to either T cells in the absence of tumor cells or T cells co-cultured in the presence of HEp2-IκB(S32AS36A) cells (Table 4). The detection of T cell depletion was seen when T cells were treated with IL-2 (Table 6). Similarly, the levels of 3H thymidine incorporation were lower in the co-cultures of T cells and vector-alone transfected HEp2 cells and not in T cell cultures which either did not receive any tumor cells or receive HEp2-IκB(S32AS36A) cells (Table 4). Differences in the thymidine incorporation between samples could be seen in IL-2 treated T cells and not in untreated T cell cultures. Furthermore, unlike NK cells, the drop-in proliferation in the presence of IFN-γ treatment was not consistently seen in the T cell cultures treated with IL-2. Collectively these data suggest that the blocking of NFKB function in tumor cells abolishes the capacity of tumor cells to mediate functional inactivation and depletion of CD8+ T cells.

TABLE 4
Increased CD8+ T cell survival, proliferation and cytokine secretion
when co-cultured in the presence of HEp2-IκB(S32AS36A) cells

| Experiment | | | | | | |
|---|---|---|---|---|---|---|
| # cell | | Control | pRcCMV | | pRcCMV/IκB( [symbol] ) | |
| survival | IL [symbol] | (no targets) | − IFN-γ | + IFN-γ | − IFN-γ | + IFN-γ |
| | − | 0 | 1.1 × 10 [symbol] | 1.2 × 10 [symbol] | 1.2 × 10 [symbol] | 1.1 × 10 [symbol] |
| | + | 8 × 10 [symbol] | 1.7 × 10 [symbol] | 6.5 × 10 [symbol] | 2.76 × 10 [symbol] | 1.92 × 10 [symbol] |
| Proliferation | − | [symbol] ± 402 | 4514 ± 1066 | 3889 ± 1268 | 8729 ± 558 | [symbol] ± 398 |
| (3H incorp) | + | 9866 ± 205 | 8937 ± 394 | 6866 ± 564 | 47189 ± 1327 | 54463 ± 442 |
| IFN-γ | − | N.D | 4.95 ± 0.69 | 2.43 ± 0.39 | 26.6 ± 3.5 | 20.65 ± 0.64 |
| secretion | + | N.D | 8.73 ± 1.17 | 2.15 ± 0.79 | 98.9 ± 4.8 | 22 ± 2.7 |

[symbol]

indicates data missing or illegible when filed

Purified CD8+ T cells were treated with and without IL-2 (500 u/ml) overnight before their co-culture in the presence and absence of IFN-γ (50) traeted pRcCMV vector control and IκB(S32AS36A) transfected HEp2 cells (E:T ratio 1:1). After 15-23 days of incubation the supernatants were removed from the T-HEp2 cell co-cultures and assayed for released cytokines by specific and sensitive ELISA. IFN-γ treated Ep2 cell transfectants were washed three times before they were added to CD8+ T cells. The number of surviving T cells were determined in after 24 days. The rate of proliferation was determined using ³H thymidine incorporation after 22 days. The p value for the differences between T cells co-cultured in the presence of vector-alone and IκB(S32AS36A) transfected HEp2 cells are less than 0.05 for all the samples tested.

When either NK cells (data not shown) or T cells were re-exposed to either vector-alone transfected HEp2 cells or HEp2-IκB(S32AS36A) cells, the only samples which had lower cytotoxicity were cultures of T cells with vector-alone transfected HEp2 cells (Table 5). T cells that were co-cultured in the presence of HEp2-IκB(S32AS36A) cells remained viable and were capable of killing either vector-alone or IκB super-repressor transfected HEp2 cells more than what was observed in the presence of T cells which received vector-alone transfected Hep2 cells (Table 5).

TABLE 5
Inhibition of HEp2 tumor cell cytotoxicity after exposure of CD8+
T cells to HEp2-pRcCMV but not pRcCMV/IκB(S32AS36A) cells

| Cytotoxicity (LU₃₀/10⁷ cells) | | | |
|---|---|---|---|
| −IFN-γ | | +IFN-γ | |
| Control | IL [symbol] | Control | IL [symbol] |

51Cr labeled HEp2-pRcCMV

| | | | | |
|---|---|---|---|---|
| CD8 + pRcCMV | 2.4 ± 1.8 | 2.3 ± 0.0 | 2.95 ± 2.6 | 3.6 ± 0.0 |
| CD8 + pRcCMV/ | 5.55 ± 0.5 | 13.3 ± 0.0 | 0.0 ± 0.0 | 5.3 ± 0.0 |
| IκB_{(S32AS36A)} | | | | |

51Cr labeled HEp2-IκB_{(S32AS36A)}

| | | | | |
|---|---|---|---|---|
| CD8 + pRcCMV | 4.2 ± 0.0 | 14.9 ± 4.8 | 3.4 ± 0.14 | 9.7 ± 5.7 |
| CD8 + pRcCMV/ | 11.9 ± 0.0 | 35.2 ± 6.6 | 10.8 ± 0.0 | 12.8 ± 0.0 |
| IκB_{(S32AS36A)} | | | | |

[symbol]

indicates data missing or illegible when filed

Treatments were carried out as described in Table 3. After 12 days of incubation the T cell-tumor cell co-cultures were washed and added to ⁵¹Cr labeled HEp2-pRcCMV and HEp2-IκB(S32AS36A) cells. After 4-6 hours of incubation at 37° C. supernatants were harvested and the levels of released ⁵¹Cr radioactivity were determined by a y counter. The p value for the difference between the co-cultures of CD8+ T cells with vector-alone transfected HEp2 cells and with IκBcs3zAs36AJ transfected HEp2 cells are less than 0.05 for all the samples tested.

### Example 21: Inhibition of NFkB in HEp2 Tumor Cells Augments DC Function and Survival and Consequently Increases the Function of Autologous Peripheral Blood Lymphocytes

The survival and function of myeloid derived DCs were increased when co-cultured in the presence of IκB(S32AS36A) but not vector-alone transfected HEp2 cells. Treatment of DCs with either Lipopolysaccharide (LPS) or IFN-γ or a combination of IFN-γ and LPS augmented the function of DCs and this increase remained higher in the presence of IκB(S32AS36A) but not vector-alone transfected HEp2 cells (Table 5). Both TNF-α (Table 6) and IL-12 (data not shown) secretion were increased when DCs were treated with Lipopolysaccharide (LPS) or IFN-γ or a combination of IFN-γ and LPS and cultured in the presence of IκB(S32AS36A) but not vector-alone transfected HEp2 cells. Consequently, the treated DCs in the presence of KB(S32AS36A but not vector-alone transfected HEp2 cells were capable of augmenting GMCSF secretion by autologous Peripheral Blood lymphocytes (Table 7).

TABLE 6
DCs co-cultured in the presence of HEp2-IκB(S32AS36A)
cells secreted increased levels of TNF-α

| | TNF-α (ng/ml) | | | |
|---|---|---|---|---|
| | | | | IFN-γ + |
| | Control | IFN-γ | LPS | LPS |
| DC | 0.13 ± 0.06 | 0.83 ± 0.56 | 20.6 ± 3.79 | 52.2 ± 4.67 |
| DC + pReCMV- | 0.13 ± 0.03 | 0.16 ± 0.01 | 7.8 | 38.65 ± 23.46 |
| HEp-2 | | | | |
| DC + HEp2- | 0.73 ± 0.6 | 0.19 ± 0.01 | 34.35 ± 1.63 | 110.4 ± 25.6 |
| IκB_{(S32AS36A)} | | | | |

DCs were treated with IFN-γ (500 u/ml), LPS (10 ng/ml) or a combination of LPS and IFN-γ and co-cultured in the presence and absence of vector-alone or IκB(S32AS36A) transfected HEp2 cells (DC:tumor ratio; 1:1). After 72 hours of incubation the supernatants were removed and the levels of TNF-α release determined by a sensitive and specific ELISA.

TABLE 7
Peripheral Blood Lymphocytes co-cultured in the presence of
HEp2-IκB(S32AS36A) cells secreted high levels of
GM-CSF in the presence of DCs

| | GM-CSF (pg/ml) | | | |
|---|---|---|---|---|
| | Control | IFN-γ | LPS | IFN-γ + LPS |
| DC + PBL | 505 ± 7.1 | 1105 ± 78 | 558.5 ± 83 | 1145 ± 64 |
| DC + PBL + | 245 ± 7 | 235 ± 7.1 | 370 ± 14 | 230 ± 0.0 |
| pRcCMV- | | | | |
| HEp-2 | | | | |
| DC + PBL + | 3965 ± 134 | 6060 ± 1230 | 9745 ± 346 | 12625 ± 318 |
| HEp2- | | | | |
| IκB_{(S32AS36A)} | | | | |

DCs were co-cultured in the presence an absence of IL-2 (500 u/ml) treated PBLs and vector-alone or IκB(S32AS36A) transfected HEp2 cells (DC:PBL:tumor ratio; 1:10:1). The treatments of DCs were carried out as described in Table 7. The levels of GM-CSF secretion were determined in the supernatants by ELISA after 72 hours. DCs alone in the presence and absence of HEp2 cell transfectants did not secrete any appreciable levels of GM-CSF (the highest amount was 18.78 pg/ml). The highest level of GM-CSF secretion obtained by PBLs was 580.13 pg/ml and treatments with IFN-γ and/or LPS did not modify the levels appreciably.

### Example 22: Increased Levels of Cytotoxicity and Cytokine Secretion by NK Cells when Exposed to Sulindac Treated Vector-Alone Transfected HEp2 Cells

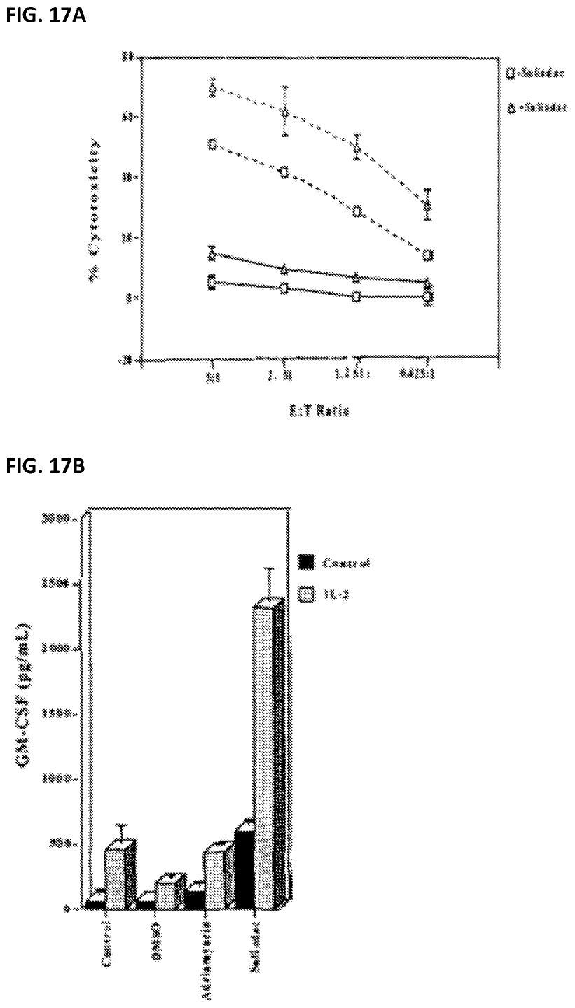

Sulindac, a non-steroidal anti-inflammatory drug is shown to inhibit NFκB function in several tumor cell lines, and it is used as a chemopreventive drug in a variety of cancers. We therefore treated vector-alone transfected HEp2 cells with Sulindac at a concentration that was not cytotoxic to HEp2 cells before their exposure to NK cells. NK cells mediated increased lysis of Sulindac treated HEp2 cells when compared to non-treated HEp2 cells (FIG. 17A). Likewise, the addition of Sulindac treated vector-alone transfected HEp2 cells to NK cells triggered increased levels of GM-CSF secretion by NK cells when compared to non-treated vector-alone transfected HEp2 cells (FIG. 17B). The levels of GM-CSF secretion were higher in Sulindac treated HEp2 cells when compared to either non-treated, DMSO treated or Adriamycin treated HEp2 cells. However, the levels of GM-CSF secretion by NK cells in the presence of Sulindac treated vector-alone transfected HEp2 cells remained inferior to that seen in the co-cultures of NK-HEp2-IκB(s32As36A) cells (data not shown).

### REFERENCES

Tseng, H. C., et al., Differential Cytotoxicity but Augmented IFN-gamma Secretion by NK Cells after Interaction with Monocytes from Humans, and Those from Wild Type and Myeloid-Specific COX-2 Knockout Mice. Front Immunol, 2015. 6: p. 259.
Cacalano, N. A., et al., Regulation of IGFBP6 gene and protein is mediated by the inverse expression and function of c-jun N-terminal kinase (INK) and NFkappaB in a model of oral tumor cells. Apoptosis, 2008. 13(12): p. 1439-49.
Fu, X. Y., STAT3 in immune responses and inflammatory bowel diseases. Cell Research, 2006. 16(2): p. 214-219.
Jewett, A., et al., Inhibition of nuclear factor kappa B (NFkappaB) activity in oral tumor cells prevents depletion of NK cells and increases their functional activation. Cancer Immunol Immunother, 2006. 55(9): p. 1052-63.
Jewett, A., et al., Cytokine dependent inverse regulation of CD54 (ICAM1) and major histocompatibility complex class I antigens by nuclear factor kappaB in HEp2 tumor cell line: effect on the function of natural killer cells. Hum Immunol, 2003. 64(5): p. 505-20.
Kozlowska, A. K., et al., Differentiation by NK cells is a prerequisite for effective targeting of cancer stem cells/poorly differentiated tumors by chemopreventive and chemotherapeutic drugs. J Cancer, 2017. 8(4): p. 537-554.
Wu, J., et al., DAP10 and DAP12 form distinct, but functionally cooperative, receptor complexes in natural killer cells. J Exp Med, 2000. 192(7): p. 1059-68.
Wu J, Cherwinski H, Spies T, Phillips J H, Lanier L L. DAP10 and DAP12 form distinct, but functionally cooperative, receptor complexes in natural killer cells. J Exp Med (2000) 192(7):1059-68. doi:10.1084/jem.192.7.1059
Jewett A, Cacalano N A, Teruel A, Romero M, Rashedi M, Wang M, et al. Inhibition of nuclear factor kappa B (NFkappaB) activity in oral tumor cells prevents depletion of NK cells and increases their functional activation. Cancer Immunol Immunother (2006) 55(9):1052-63. doi:10.1007/s00262-005-0093-7
Jewett A, Wang M Y, Teruel A, Poupak Z, Bostanian Z, Park N H. Cytokine dependent inverse regulation of CD54 (ICAM1) and major histocompatibility complex class I antigens by nuclear factor kappaB in HEp2 tumor cell line: effect on the function of natural killer cells. Hum Immunol (2003) 64(5):505-20. doi:10.1016/S0198-8859(03)00039-9
Tato C M, Mason N, Artis D, Shapira S, Caamano J C, Bream J H, et al. Opposing roles of NF-kappaB family members in the regulation of NK cell proliferation and production of IFN-gamma. Int Immunol (2006) 18(4):505-13. doi:10.1093/intimm/dxh391
Bedel R, Thiery-Vuillemin A, Grandclement C, Balland J, Remy-Martin J P, Kantelip B, et al. Novel role for STAT3 in transcriptional regulation of NK immune cell targeting receptor MICA on cancer cells. Cancer Res (2011) 71(5):1615-26. doi:10.1158/0008-5472.CAN-09-4540
Ihara S, Kida H, Arase H, Tripathi L P, Chen Y A, Kimura T, et al. Inhibitory roles of signal transducer and activator of transcription 3 in antitumor immunity during carcinogen-induced lung tumorigenesis. Cancer Res (2012) 72(12):2990-9. doi:10.1158/0008-5472.CAN-11-4062
Kortylewski M, Kujawski M, Wang T, Wei S, Zhang S, Pilon-Thomas S, et al. Inhibiting Stat3 signaling in the hematopoietic system elicits multicomponent antitumor immunity. Nat Med (2005) 11(12):1314-21. doi:10.1038/nm1325
Kohga K, Tatsumi T, Takehara T, Tsunematsu H, Shimizu S, Yamamoto M, et al. Expression of CD133 confers malignant potential by regulating metalloproteinases in human hepatocellular carcinoma. J Hepatol (2010) 52(6):872-9. doi:10.1016/j.jhep.2009.12.030
Beraza N, Malato Y, Sander L E, Al-Masaoudi M, Freimuth J, Riethmacher D, et al. Hepatocyte-specific NEMO deletion promotes NK/NKT cell- and TRAILdependent liver damage. J Exp Med (2009) 206(8):1727-37. doi:10.1084/jem. 20082152
Orange J S, Brodeur S R, Jain A, Bonilla F A, Schneider L C, Kretschmer R, et al. Deficient natural killer cell cytotoxicity in patients with IKK-gamma/NEMO mutations. J Clin Invest (2002) 109(11):1501-9. doi:10.1172/JC114858
Pachlopnik Schmid J M, Junge S A, Hossle J P, Schneider E M, Roosnek E, Seger R A, et al. Transient hemophagocytosis with deficient cellular cytotoxicity, monoclonal immunoglobulin M gammopathy, increased T-cell numbers, and hypomorphic NEMO mutation. Pediatrics (2006) 117(5):e1049-56. doi:10. 1542/peds.2005-2062
Romero-Reyes M, Head C, Cacalano N A, Jewett A. Potent induction of TNFalpha during interaction of immune effectors with oral tumors as a potential mechanism for the loss of NK cell viability and function. Apoptosis (2007) 12(11):2063-75. doi:10.1007/s10495-007-0112-6
Alex P, Ye M, Zachos N C, Sipes J, Nguyen T, Suhodrev M, et al. Clcn5 knockout mice exhibit novel immunomodulatory effects and are more susceptible to dextran sulfate sodium-induced colitis. J Immunol (2010) 184(7):3988-96. doi:10.4049/jimmunol.0901657
Thompson W L, Karpus W J, Van Eldik U. MCP-1-deficient mice show reduced neuroinflammatory responses and increased peripheral inflammatory responses to peripheral endotoxin insult. J Neuroinflammation (2008) 5:35. doi:10.1186/1742-2094-5-35
Bognar P, Nemeth I, Mayer B, Haluszka D, Wikonkal N, Ostorhazi E, et al. Reduced inflammatory threshold indicates skin barrier defect in transglutaminase 3 knockout mice. J Invest Dermatol (2014) 134(1):105-11. doi:10.1038/jid. 2013.307
Beglopoulos V, Sun X, Saura C A, Lemere C A, Kim R D, Shen J. Reduced beta-amyloid production and increased inflammatory responses in presenilin conditional knock-out mice. J Biol Chem (2004) 279(45):46907-14. doi:10. 1074/jbc.M409544200
Roviezzo F, Getting S J, Paul-Clark M J, Yona S, Gavins F N, Perretti M, et al. The annexin-1 knockout mouse: what it tells us about the inflammatory response. J Physiol Pharmacol (2002) 53(4 Pt 1):541-53.
Guedes R P, Csizmadia E, Moll H P, Ma A, Ferran C, da Silva C G. A20 deficiency causes spontaneous neuroinflammation in mice. J Neuroinflammation (2014) 11:122. doi:10.1186/1742-2094-11-122
Radosavljevic G, Jovanovic I, Majstorovic I, Mitrovic M, Lisnic V J, Arsenijevic N, et al. Deletion of galectin-3 in the host attenuates metastasis of murine melanoma by modulating tumor adhesion and NK cell activity. Clin Exp Metastasis (2011) 28(5):451-62. doi:10.1007/s10585-011-9383-y
Handschin C, Chin S, Li P, Liu F, Maratos-Flier E, Lebrasseur N K, et al. Skeletal muscle fiber-type switching, exercise intolerance, and myopathy in PGC-1alpha muscle-specific knock-out animals. J Biol Chem (2007) 282(41):30014-21. doi:10.1074/jbc.M704817200
Bieghs V, Van Gorp P J, Wouters K, Hendrikx T, Gijbels M J, van Bilsen M, et al. LDL receptor knock-out mice are a physiological model particularly vulnerable to study the onset of inflammation in non-alcoholic fatty liver disease. PLoS One (2012) 7(1):e30668. doi:10.1371/journal.pone.0030668
Zhu X, Lee J Y, Timmins J M, Brown J M, Boudyguina E, Mulya A, et al. Increased cellular free cholesterol in macrophage-specific Abca1 knock-out mice enhances proinflammatory response of macrophages. J Biol Chem (2008) 283(34):22930-41. doi:10.1074/jbc.M801408200
Barta P, Van Pelt C, Men T, Dickey B F, Lotan R, Moghaddam S J. Enhancement of lung tumorigenesis in a Gprc5a Knockout mouse by chronic extrinsic airway inflammation. Mol Cancer (2012) 11:4. doi:10.1186/1476-4598-11-4
van Helden Y G, Heil S G, van Schooten F J, Kramer E, Hessel S, Amengual J, et al. Knockout of the Bcmol gene results in an inflammatory response in female lung, which is suppressed by dietary beta-carotene. Cell Mol Life Sci (2010) 67(12):2039-56. doi:10.1007/s00018-010-0341-7
Gironella M, Folch-Puy E, LeGoffic A, Garcia S, Christa L, Smith A, et al. Experimental acute pancreatitis in PAP/HIP knock-out mice. Gut (2007) 56(8):1091-7. doi:10.1136/gut.2006.116087
Chu S, Tanaka S, Kaunitz J D, Montrose M H. Dynamic regulation of gastric surface pH by luminal pH. J Clin Invest (1999) 103(5):605-12. doi:10.1172/JC15217
Garner A, Flemström G, Allen A, Heylings J R, McQueen S. Gastric mucosal protective mechanisms: roles of epithelial bicarbonate and mucus secretions. Scand J Gastroenterol Suppl (1984) 101:79-86.
Bergstrom S, Duner H, von Euler U, Pernow B, Sjovall J. Observations on the effects of infusion of prostaglandin E in man. Acta Physiol Scand (1959) 45:145-51. doi:10.1111/j.1748-1716.1959.tb01686.x
Solomon L M, Juhlin L, Kirschenbaum M B. Prostaglandin on cutaneous vasculature. J Invest Dermatol (1968) 51(4):280-2. doi:10.1038/jid.1968.46
Satoh T, Moroi R, Aritake K, Urade Y, Kanai Y, Sumi K, et al. Prostaglandin D2 plays an essential role in chronic allergic inflammation of the skin via CRTH2 receptor. J Immunol (2006) 177(4):2621-9. doi:10.4049/jimmunol.177. 4.2621
Gavett S H, Madison S L, Chulada P C, Scarborough P E, Qu W, Boyle J E, et al. Allergic lung responses are increased in prostaglandin H synthase-deficient mice. J Clin Invest (1999) 104(6):721-32. doi:10.1172/JC16890
Weiss H J, Turitto V T. Prostacyclin (prostaglandin 12, PGI2) inhibits platelet adhesion and thrombus formation on subendothelium. Blood (1979) 53(2):244-50.
Paul B Z, Jin J, Kunapuli S P. Molecular mechanism of thromboxane A(2)-induced platelet aggregation. Essential role for p2t(ac) and alpha(2a) receptors. J Biol Chem (1999) 274(41):29108-14. doi:10.1074/jbc.274.41.29108
Syeda M M, Jing X, Mirza R H, Yu H, Sellers R S, Chi Y. Prostaglandin transporter modulates wound healing in diabetes by regulating prostaglandin induced angiogenesis. Am J Pathol (2012) 181(1):334-46. doi:10.1016/j.ajpath. 2012.03.012
Iwanaga K, Okada M, Murata T, Hori M, Ozaki H. Prostaglandin E2 promotes wound-induced migration of intestinal subepithelial myofibroblasts via EP2, EP3, and EP4 prostanoid receptor activation. J Pharmacol Exp Ther (2012) 340(3):604-11. doi:10.1124/jpet. 111.189845
Prisk V, Huard J. Muscle injuries and repair: the role of prostaglandins and inflammation. Histol Histopathol (2003) 18(4):1243-56.
Kramer H J, Glanzer K, Dusing R. Role of prostaglandins in the regulation of renal water excretion. Kidney Int (1981) 19(6):851-9. doi:10.1038/ki.1981.89
Jackson L M, Wu K C, Mahida Y R, Jenkins D, Hawkey C J. Cyclooxygenase (COX) 1 and 2 in normal, inflamed, and ulcerated human gastric mucosa. Gut (2000) 47(6):762-70. doi:10.1136/gut.47.6.762
Peskar B M. Role of cyclooxygenase isoforms in gastric mucosal defence. J Physiol Paris (2001) 95(1-6):3-9. doi:10.1016/S0928-4257(01)00003-1
Otto J C, Smith W L. Prostaglandin endoperoxide synthases-1 and -2. J Lipid Mediat Cell Signal (1995) 12(2-3):139-56. doi:10.1016/0929-7855(95)00015-I
Herschman H R. Prostaglandin synthase 2. Biochim Biophys Acta (1996) 1299(1):125-40. doi:10.1016/0005-2760(95)00194-8
Mao X Y, Wang X G, Lv X J, Xu L, Han C B. COX-2 expression in gastric cancer and its relationship with angiogenesis using tissue microarray. World J Gastroenterol (2007) 13(25):3466-71. doi:10.3748/wjg.v13.i25.3466
Lazar D, Taban S, Ardeleanu C, Simionescu C, Sporea I, Cornianu M, et al. Immunohistochemical expression of the cyclooxygenase-2 (COX-2) in gastric cancer. The correlations with the tumor angiogenesis and patients' survival. Rom J Morphol Embryol (2008) 49(3):371-9.
Gou H F, Chen X C, Zhu J, Jiang M, Yang Y, Cao D, et al. Expressions of COX-2 and VEGF-C in gastric cancer: correlations with lymphangiogenesis and prognostic implications. J Exp Clin Cancer Res (2011) 30:14. doi:10.1186/1756-9966-30-14
Tseng H C, Arasteh A, Paranjpe A, Teruel A, Yang W, Behel A, et al. Increased lysis of stem cells but not their differentiated cells by natural killer cells de-differentiation or reprogramming activates NK cells. PLoS One (2010) 5(7):e11590. doi:10.1371/journal.pone.0011590
Whiteside T L, Herberman R B. The role of natural killer cells in immune surveillance of cancer. Curr Opin Immunol (1995) 7(5):704-10. doi:10.1016/0952-7915(95)80080-8
Jewett A, Bonavida B. Target-induced anergy of natural killer cytotoxic function is restricted to the NK-target conjugate subset. Cell Immunol (1995) 160(1):91-7. doi:10.1016/0008-8749(95)80013-9
Jewett A, Bonavida B. Target-induced inactivation and cell death by apoptosis in a subset of human NK cells. J Immunol (1996) 156(3):907-15.
Jewett A, Gan X H, Lebow L T, Bonavida B. Differential secretion of TNFalpha and IFN-gamma by human peripheral blood-derived NK subsets and association with functional maturation. J Clin Immunol (1996) 16(1):46-54. doi:10.1007/BF01540972
Jewett A, Cavalcanti M, Bonavida B. Pivotal role of endogenous TNF-alpha in the induction of functional inactivation and apoptosis in NK cells. J Immunol (1997) 159:4815-22.
Jewett A, Bonavida B. MHC-Class I antigens regulate both the function and the survival of human peripheral blood NK cells: role of endogenously secreted TNF-alpha. Clin Immunol (2000) 96(1):19-28. doi:10.1006/clim.2000. 4871
Jewett A, Cacalano N A, Head C, Teruel A. Coengagement of CD16 and CD94 receptors mediates secretion of chemokines and induces apoptotic death of naive natural killer cells. Clin Cancer Res (2006) 12(7 Pt 1):1994-2003. doi:10. 1158/1078-0432.CCR-05-2306
Jewett A, Teruel A, Romero M, Head C, Cacalano N. Rapid and potent induction of cell death and loss of NK cell cytotoxicity against oral tumors by F(ab′)2 fragment of anti-CD16 antibody. Cancer Immunol Immunother (2008) 57(7):1053-66. doi:10.1007/s00262-007-0437-6
Jewett A, Man Y G, Cacalano N, Kos J, Tseng H C. Natural killer cells as effectors of selection and differentiation of stem cells: role in resolution of inflammation. J
Immunotoxicol (2014) 11(4):297-307. doi:10.3109/1547691X. 2013.877104 Jewett A, Kos J, Kaur K, Tumsek T, Breznik B, Senjor E, Wong P, Nguyen K, Ko M. Multiple Defects of Natural Killer Cells in Cancer Patients: Anarchy, Dysregulated Systemic Immunity, and Immunosuppression in Metastatic Cancer (2020) Critical Reviews in Immunology, 40(2):93-133
Kaur K, Kozlowska A, Topchyan P, Ko M, Ohanian N, Chiang J, Cook J, Maung P, Park S, Cacalano C, Fang C, Jewett A. Probiotic—Treated Super—Charged NK Cells Efficiently Clear Poorly Differentiated Pancreatic Tumors in Hu—BLT Mice (2020) Cancer 12(63)1-23
Kaur K, Nanut M, Ko M, Safaie T, Kos J, Jewett A. Natural killer cells target and differentiate cancer stem-like cells/undifferentiated tumors: strategies to optimize their growth and expansion for effective cancer immunotherapy (2018) Current Opinion in Immunology 51:170-180
Jewett A, Kos J, Kaur K, Safaei T, Sutanto, Chen W, Wong P, Namagerdi A, Fang C, Fong Y, Ko M. Natural Killer Cells: Diverse Functions in Tumor Immunity and Defects in Pre-neoplastic and Neoplastic Stages of Tumorigenesis (2020) Molecular Therapy: Oncolytics 16:41-52
Jewett A, Man Y G, Tseng H C. Dual functions of natural killer cells in selection and differentiation of stem cells; role in regulation of inflammation and regeneration of tissues. J Cancer (2013) 4(1):12-24. doi:10.7150/jca. 5519
Jewett A, Tseng H C. Potential rescue, survival and differentiation of cancer stem cells and primary non-transformed stem cells by monocyte-induced split anergy in natural killer cells. Cancer Immunol Immunother (2012) 61(2):265-74. doi:10.1007/s00262-011-1163-7
Jewett A, Tseng H C. Tumor induced inactivation of natural killer cell cytotoxic function; implication in growth, expansion and differentiation of cancer stem cells. J Cancer (2011) 2:443-57. doi:10.7150/jca.2.443
Goodier M R, Londei M. Lipopolysaccharide stimulates the proliferation of human CD56+θCD3− NK cells: a regulatory role of monocytes and IL-10. J Immunol (2000) 165(1):139-47. doi:10.4049/jimmunol.165.1.139
Mian M F, Lauzon N M, Andrews D W, Lichty B D, Ashkar A A. FimH can directly activate human and murine natural killer cells via TLR4. Mol Ther (2010) 18(7):1379-88. doi:10.1038/mt.2010.75
Tseng H C, Bui V, Man Y G, Cacalano N, Jewett A. Induction of split anergy conditions natural killer cells to promote differentiation of stem cells through Cell-Cell contact and secreted factors. Front Immunol (2014) 5:269. doi:10.3389/fimmu.2014.00269
Jewett A, Tseng H C, Arasteh A, Saadat S, Christensen R E, Cacalano N A. Natural killer cells preferentially target cancer stem cells; role of monocytes in protection against NK cell mediated lysis of cancer stem cells. Curr Drug Deliv (2012) 9(1):5-16. doi:10.2174/156720112798375989
Ishikawa T O, Herschman H R. Conditional knockout mouse for tissue-specific disruption of the cyclooxygenase-2 (Cox-2) gene. Genesis (2006) 44(3):143-9. doi:10.1002/gene.20192
Pasparakis M, Courtois G, Hafner M, Schmidt-Supprian M, Nenci A, Toksoy A, et al. TNF-mediated inflammatory skin disease in mice with epidermisspecific deletion of IKK2. Nature (2002) 417(6891):861-6. doi:10.1038/nature00820
Karin M, Cao Y, Greten F R, Li Z W. NF-kappaB in cancer: from innocent bystander to major culprit. Nat Rev Cancer (2002) 2(4):301-10. doi:10.1038/nrc780
Jewett A, Tseng H C. Tumor microenvironment may shape the function and phenotype of NK cells through the induction of split anergy and generation of regulatory NK cells. In: Shurin M, Umansky V, Malyguine A, editors. The Tumor Immunoenvironment. Dordrecht: Springer (2013). p. 361-84.
Jewett A, Arasteh A, Tseng H C, Behel A, Arasteh H, Yang W, et al. Strategies to rescue mesenchymal stem cells (MSCs) and dental pulp stem cells (DPSCs) from NK cell mediated cytotoxicity. PLoS One (2010) 5(3):e9874. doi:10.1371/journal.pone.0009874
Kanevskiy L M, Telford W G, Sapozhnikov A M, Kovalenko E L. Lipopolysaccharide induces IFN-gamma production in human NK cells. Front Immunol (2013) 4:11. doi:10.3389/fimmu.2013.00011
O'Connor G M, Hart O M, Gardiner C M. Putting the natural killer cell in its place. Immunology (2006) 117(1):1-10. doi:10.1111/j.1365-2567.2005.02256.x
Shinkai, Y., et al., RAG-2-deficient mice lack mature lymphocytes owing to inability to initiate V(D)J rearrangement. Cell, 1992. 68(5): p. 855-67.
Golumbek, P. T., R. M. Keeling, and A. M. Connolly, RAG2 gene knockout in mice causes fatigue. Muscle Nerve, 2007. 36(4): p. 471-6.
Mombaerts, P., et al., RAG-1-deficient mice have no mature B and T lymphocytes. Cell, 1992. 68(5): p. 869-77.
Spanopoulou, E., et al., Functional immunoglobulin transgenes guide ordered B-cell differentiation in Rag-1-deficient mice. Genes Dev, 1994. 8(9): p. 1030-42.
Grawunder, U., et al., Down-regulation of RAG1 and RAG2 gene expression in preB cells after functional immunoglobulin heavy chain rearrangement. Immunity, 1995. 3(5):601-8.
Yu, W., et al., Coordinate regulation of RAG1 and RAG2 by cell type-specific DNA elements 5′ of RAG2. Science, 1999. 285(5430): p. 1080-4.
Kondo, M., I. L. Weissman, and K. Akashi, Identification of clonogenic common lymphoid progenitors in mouse bone marrow. Cell, 1997. 91(5): p. 661-72.
Borghesi, L., et al., B lineage-specific regulation of V(D)J recombinase activity is established in common lymphoid progenitors. J Exp Med, 2004. 199(4): p. 491-502.
Veinotte, L. L., et al., Expression of rearranged TCRgamma genes in natural killer cells suggests a minor thymus-dependent pathway of lineage commitment. Blood, 2006. 107(7): p. 2673-9.
Fronkova, E., et al., Lymphoid differentiation pathways can be traced by TCR delta rearrangements. J Immunol, 2005. 175(4): p. 2495-500.
Karo, J. M., D. G. Schatz, and J. C. Sun, The RAG recombinase dictates functional heterogeneity and cellular fitness in natural killer cells. Cell, 2014. 159(1): p. 94-107.
Pilbeam, K., et al., The ontogeny and fate of NK cells marked by permanent DNA rearrangements. J Immunol, 2008. 180(3): p. 1432-41.
Palmer, J. M., et al., Clinical relevance of natural killer cells following hematopoietic stem cell transplantation. J Cancer, 2013. 4(1): p. 25-35.
Kaur, K., et al., Suppression of Gingival NK Cells in Precancerous and Cancerous Stages of Pancreatic Cancer in KC and BLT-Humanized Mice. Front Immunol, 2017. 8: p. 1606.
Fildes, J. E., N. Yonan, and C. T. Leonard, Natural killer cells and lung transplantation, roles in rejection, infection, and tolerance. Transpl Immunol, 2008. 19(1): p. 1-11.
Farag, S. S. and M. A. Caligiuri, Human natural killer cell development and biology. Blood Rev, 2006. 20(3): p. 123-37.
Kaur, K., et al., Natural killer cells target and differentiate cancer stem-like cells/undifferentiated tumors: strategies to optimize their growth and expansion for effective cancer immunotherapy. Current Opinion in Immunology, 2018. 51: p. 170-180.
Kaur, K., et al., Novel Strategy to Expand Super-Charged NK Cells with Significant Potential to Lyse and Differentiate Cancer Stem Cells: Differences in NK Expansion and Function between Healthy and Cancer Patients. Front Immunol, 2017. 8: p. 297.
Bui, V. T., et al., Augmented IFN-gamma and TNF-alpha Induced by Probiotic Bacteria in NK Cells Mediate Differentiation of Stem-Like Tumors Leading to Inhibition of Tumor Growth and Reduction in Inflammatory Cytokine Release; Regulation by IL-10. Front Immunol, 2015. 6: p. 576.
Jewett, A., et al., Natural killer cells preferentially target cancer stem cells; role of monocytes in protection against NK cell mediated lysis of cancer stem cells. Curr Drug Deliv, 2012. 9(1): p. 5-16.
Soderstrom, K., et al., Natural killer cells trigger osteoclastogenesis and bone destruction in arthritis. Proc Natl Acad Sci USA, 2010. 107(29): p. 13028-33.
Takayanagi, H., et al., T-cell-mediated regulation of osteoclastogenesis by signalling cross-talk between RANKL and IFN-gamma. Nature, 2000. 408(6812): p. 600-5.
Gao, Y., et al., IFN-gamma stimulates osteoclast formation and bone loss in vivo via antigen-driven T cell activation. J Clin Invest, 2007. 117(1): p. 122-32.
Hofbauer, L. C., et al., Interleukin-Ibeta and tumor necrosis factor-alpha, but not interleukin-6, stimulate osteoprotegerin ligand gene expression in human osteoblastic cells. Bone, 1999. 25(3): p. 255-9.
Lam, J., et al., TNF-alpha induces osteoclastogenesis by direct stimulation of macrophages exposed to permissive levels of RANK ligand. J Clin Invest, 2000. 106(12): p. 1481-8.
Russell, R. G., et al., Mechanisms of action of bisphosphonates: similarities and differences and their potential influence on clinical efficacy. Osteoporos Int, 2008. 19(6):733-59.
Ito, M., et al., Ultrastructural and cytochemical studies on cell death of osteoclasts induced by bisphosphonate treatment. Bone, 1999. 25(4): p. 447-52.
Breuil, V., et al., Human osteoclast formation and activity in vitro: effects of alendronate. J Bone Miner Res, 1998. 13(11): p. 1721-9.
Hughes, D. E., et al., Bisphosphonates promote apoptosis in murine osteoclasts in vitro and in vivo. J Bone Miner Res, 1995. 10(10): p. 1478-87.
Selander, K. S., et al., Characteristics of clodronate-induced apoptosis in osteoclasts and macrophages. Mol Pharmacol, 1996. 50(5): p. 1127-38.
Tseng, H. C., et al., Bisphosphonate-induced differential modulation of immune cell function in gingiva and bone marrow in vivo: role in osteoclast-mediated NK cell activation. Oncotarget, 2015. 6(24): p. 20002-25.
Rasmusson, L. and J. Abtahi, Bisphosphonate associated osteonecrosis of the jaw: an update on pathophysiology, risk factors, and treatment. Int J Dent, 2014. 2014: p. 471035.
Khosla, S., et al., Bisphosphonate-associated osteonecrosis of the jaw: report of a task force of the American Society for Bone and Mineral Research. J Bone Miner Res, 2007. 22(10): p. 1479-91.
Allen, M. R. and S. L. Ruggiero, A review of pharmaceutical agents and oral bone health: how osteonecrosis of the jaw has affected the field. Int J Oral Maxillofac Implants, 2014. 29(1): p. e45-57.
Estilo, C. L., et al., Osteonecrosis of the maxilla and mandible in patients with advanced cancer treated with bisphosphonate therapy. Oncologist, 2008. 13(8): p. 911-20.
Fedele, S., et al., Nonexposed variant of bisphosphonate-associated osteonecrosis of the jaw: a case series. Am J Med, 2010. 123(11): p. 1060-4.
Marx, R. E., Pamidronate (Aredia) and zoledronate (Zometa) induced avascular necrosis of the jaws: a growing epidemic. J Oral Maxillofac Surg, 2003. 61(9): p. 1115-7.
Favus, M. J., Diabetes and the risk of osteonecrosis of the jaw. J Clin Endocrinol Metab, 2007. 92(3): p. 817-8.
Palaska, P. K., V. Cartsos, and A. I. Zavras, Bisphosphonates and time to osteonecrosis development. Oncologist, 2009. 14(11): p. 1154-66.
King, A. E. and E. M. Umland, Osteonecrosis of the jaw in patients receiving intravenous or oral bisphosphonates. Pharmacotherapy, 2008. 28(5): p. 667-77.
Jackson, G. H., et al., Osteonecrosis of the jaw and renal safety in patients with newly diagnosed multiple myeloma: Medical Research Council Myeloma IX Study results. Br J Haematol, 2014. 166(1): p. 109-17.
Woo, S. B., J. W. Hellstein, and J. R. Kalmar, Narrative [corrected] review: bisphosphonates and osteonecrosis of the jaws. Ann Intern Med, 2006. 144(10): p. 753-61.
Saad, F., et al., Incidence, risk factors, and outcomes of osteonecrosis of the jaw: integrated analysis from three blinded active-controlled phase III trials in cancer patients with bone metastases. Ann Oncol, 2012. 23(5): p. 1341-7.
Ting, K., et al., A short isoform of Col9aa supports alveolar bone repair. Am J Pathol, 1999. 155(6): p. 1993-9.
Jewett, A. and B. Bonavida, Interferon-alpha activates cytotoxic function but inhibits interleukin-2-mediated proliferation and tumor necrosis factor-alpha secretion by immature human natural killer cells. J Clin Immunol, 1995. 15(1): p. 35-44.
Jewett, A. and B. Bonavida, Target-induced inactivation and cell death by apoptosis in a subset of human NK cells. J Immunol, 1996. 156(3): p. 907-15.
Jewett, A., et al., Cytokine dependent inverse regulation of CD54 (ICAM1) and major histocompatibility complex class I antigens by nuclear factor kappaB in HEp2 tumor cell line: effect on the function of natural killer cells. Hum Immunol, 2003. 64(5): p. 505-20.
Regunathan, J., et al., NKG2D receptor-mediated NK cell function is regulated by inhibitory Ly49 receptors. Blood, 2005. 105(1): p. 233-40.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A cancer vaccine comprising cancer cells, wherein the cancer cells have a decreased copy number, amount, and/or activity of at least one biomarker that is a marker of differentiation of the cancer cells.

2. A cancer vaccine comprising cancer cells, wherein the cancer cells have a decreased copy number, amount, and/or activity of at least one biomarker selected from: Cox2, Rag2, NFkB, STAT3, CD133, NEMO, TNF-a, DAP10, DAP12, Clc-5, MCP-1, Transglutaminase 3, Presenilin 1, Presenilin 2, Annexin-1, A20 (TNFAIP3), Galectin-1, Galectin-3, PGC-1a, LDLR, Abca1, Gprc5a, BCMO1, PAP/HIP, ApoE, IL-6, IL-8, GSK3, ADAM17, NKG2A, CD3 zeta, MCT4, c-CBL, EZH2, SUP-1, and Cbl-b.

3. The cancer vaccine of claim 1 or 2, wherein the cancer cells are irradiated, optionally wherein the cancer cells are gamma irradiated.

4. The cancer vaccine of any one of claims 1-3, wherein the cancer cells have a decreased copy number, amount, and/or activity of Cox2, Rag2, and/or NFkB.

5. The cancer vaccine of any one of claims 1-4, wherein the cancer cells have a decreased copy number, amount, and/or activity of Cox2.

6. The cancer vaccine of any one of claims 1-4, wherein the cancer cells have a decreased copy number, amount, and/or activity of Rag2.

7. The cancer vaccine of any one of claims 1-6, wherein the copy number, amount, and/or activity of at least one biomarker is decreased by contacting the cancer cells with a small molecule inhibitor, CRISPR guide RNA (gRNA), RNA interfering agent, antisense oligonucleotide, peptide or peptidomimetic inhibitor, aptamer, antibody, and/or intrabody.

8. The cancer vaccine of any one of claims 1-7, wherein the cancer vaccine activates an NK cell.

9. The cancer vaccine of claim 8, wherein the NK cell is a primary NK cell.

10. The cancer vaccine of claim 8 or 9, wherein the cancer vaccine expands the NK cell, optionally wherein the NK cell expands to at least about 10, 11, 12, 13, 14, 15, 16, 17, 18, 19, 20, 21, 22, 23, 24, 25, or more population doublings within 4 weeks.

11. The cancer vaccine of any one of claims 1-10, wherein the cancer vaccine enhances NK cell cytotoxicity.

12. The cancer vaccine of any one of claims 1-11, wherein the cancer vaccine increases or promotes production, secretion, and/or function of at least one cytokine or chemokine produced by the NK cell.

13. The cancer vaccine of claim 12, wherein the at least one cytokine is IFN-γ.

14. The cancer vaccine of any one of claims 8-13, wherein the NK cell expands a CD8+ T cell.

15. The cancer vaccine of claim 14, wherein the NK cell preferentially expands the CD8+ T cell relative to a CD4+ T cell.

16. The cancer vaccine of any one of claims 1-15, further comprising one or more additional agents capable of activating an NK cell, or enhancing secretion of IFN-γ by an NK cell.

17. The cancer vaccine of claim 16, wherein the one or more additional agents are selected from IL-2, anti-CD16 antibody, anti-CD3 antibody, anti-CD28 antibody, and a composition comprising at least one bacterial strain.

18. The cancer vaccine of any one of claims 1-17, further comprising at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the at least one bacterial strain is sonicated.

19. The cancer vaccine of claim 18, wherein the at least one bacterial strain comprises: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the bacterial strains are sonicated.

20. The cancer vaccine of any one of claims 1-19, wherein the cancer cells are derived from a solid or hematological cancer.

21. The cancer vaccine of any one of claims 1-20, wherein the cancer cells are derived from a cancer cell line.

22. The cancer vaccine of any one of claims 1-20, wherein the cancer cells are derived from primary cancer cells.

23. The cancer vaccine of any one of claims 1-22, wherein the cancer cells are derived from multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and/or glioblastoma.

24. The cancer vaccine of any one of claims 1-23, wherein the cancer cells are derived from oral cancer, pancreatic cancer, lung cancer, colon cancer, melanoma, breast cancer, ovarian cancer, and/or glioblastoma.

25. A pharmaceutical composition comprising the cancer vaccine of any one of claims 1-24.

26. A pharmaceutical composition comprising a monocyte and/or an osteoclast, wherein the monocyte and/or the osteoclast has a decreased copy number, amount, and/or activity of at least one biomarker selected from: Cox2, Rag2, STAT3, and TNF-a.

27. The pharmaceutical composition of claim 26, wherein the monocyte and/or osteoclast is irradiated, optionally wherein the monocyte and/or osteoclast is gamma irradiated.

28. The pharmaceutical composition vaccine of claim 26 or 27, wherein the monocyte and/or osteoclast has a decreased copy number, amount, and/or activity of TNF-a or STAT3.

29. The pharmaceutical composition of any one of claims 26-28, wherein the monocyte and/or osteoclast has a decreased copy number, amount, and/or activity of Cox2.

30. The pharmaceutical composition of any one of claims 26-28, wherein the monocyte and/or osteoclast has a decreased copy number, amount, and/or activity of Rag2.

31. The pharmaceutical composition of any one of claims 26-30, wherein the copy number, amount, and/or activity of at least one biomarker is decreased by contacting the cancer cells with a small molecule inhibitor, CRISPR guide RNA (gRNA), RNA interfering agent, antisense oligonucleotide, peptide or peptidomimetic inhibitor, aptamer, antibody, and/or intrabody.

32. The pharmaceutical composition of any one of claims 26-31, wherein the pharmaceutical composition activates an NK cell.

33. The pharmaceutical composition of claim 32, wherein the NK cell is a primary NK cell.

34. The pharmaceutical composition of claim 32 or 33, wherein the pharmaceutical composition expands the NK cell, optionally wherein the NK cell expands to at least about 10, 11, 12, 13, 14, 15, 16, 17, 18, 19, 20, 21, 22, 23, 24, 25, or more population doublings within 4 weeks.

35. The pharmaceutical composition of any one of claims 26-34, wherein the pharmaceutical composition enhances NK cell cytotoxicity.

36. The pharmaceutical composition of any one of claims 26-35, wherein the pharmaceutical composition increases or promotes production, secretion, and/or function of at least one cytokine or chemokine produced by the NK cell.

37. The pharmaceutical composition of claim 36, wherein the at least one cytokine is IFN-γ.

38. The pharmaceutical composition of any one of claims 32-37, wherein the NK cell expands a CD8+ T cell.

39. The pharmaceutical composition of claim 38, wherein the NK cell preferentially expands the CD8+ T cell relative to a CD4+ T cell.

40. The pharmaceutical composition of any one of claims 26-39, further comprising one or more additional agents capable of activating an NK cell, or enhancing secretion of IFN-γ by an NK cell.

41. The pharmaceutical composition of claim 40, wherein the one or more additional agents are selected from IL-2, anti-CD16 antibody, anti-CD3 antibody, anti-CD28 antibody, and a composition comprising at least one bacterial strain.

42. The pharmaceutical composition of any one of claims 26-41, further comprising at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the at least one bacterial strain is sonicated.

43. The pharmaceutical composition of claim 42, wherein the at least one bacterial strain comprises: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the bacterial strains are sonicated.

44. The pharmaceutical composition of any one of claims 26-43, further comprising the cancer vaccine or the pharmaceutical composition of any one of claims 1-25.

45. A method of preventing or treating a cancer in a subject comprising administering to the subject:
a) the cancer vaccine or the pharmaceutical composition of any one of claims 1-44; and/or
b) NK cells activated by contacting the NK cells with the cancer vaccine or the pharmaceutical composition of any one of claims 1-44.

46. The method of claim 43, wherein the method comprises administering to the subject:
a) the cancer vaccine or the pharmaceutical composition of any one of claims 1-44; and
b) NK cells activated by contacting the NK cells with the cancer vaccine or the pharmaceutical composition of any one of claims 1-44.

47. The method of claim 45 or 46, wherein the method is part of an adjuvant therapy.

48. The method of any one of claims 45-47, wherein the cancer cells in the cancer vaccine are derived from a cancer of the same type as the cancer treated with the cancer vaccine.

49. The method of any one of claims 45-47, wherein the cancer cells in the cancer vaccine are derived from a cancer of a different type than the cancer treated with the cancer vaccine.

50. The method of any one of claims 45-49, wherein the cancer vaccine and/or the pharmaceutical composition is syngeneic or xenogeneic to the subject.

51. The method of any one of claims 45-50, wherein the cancer vaccine and/or the pharmaceutical composition is autologous, matched allogeneic, mismatched allogeneic, or congenic to the subject.

52. The method of any one of claims 45-51, wherein the cancer vaccine and/or the pharmaceutical composition activate an NK cell.

53. The method of claim 52, wherein the NK cell is a primary NK cell.

54. The method of claim 52 or 53, wherein the cancer vaccine and/or the pharmaceutical composition expands the NK cell, optionally wherein the NK cell expands to at least about 10, 11, 12, 13, 14, 15, 16, 17, 18, 19, 20, 21, 22, 23, 24, 25, or more population doublings within 4 weeks.

55. The method of any one of claims 45-54, wherein the cancer vaccine and/or the pharmaceutical composition enhances NK cell cytotoxicity.

56. The method of any one of claims 45-55, wherein the cancer vaccine and/or the pharmaceutical composition increases or promotes production, secretion, and/or function of at least one cytokine or chemokine produced by the NK cell.

57. The method of claim 56, wherein the at least one cytokine is IFN-γ.

58. The method of any one of claims 52-57, wherein the NK cell expands a CD8+ T cell.

59. The method of claim 58, wherein the NK cell preferentially expands the CD8+ T cell relative to a CD4+ T cell.

60. The method of any one of claims 45-59, wherein the cancer vaccine and/or the pharmaceutical composition is administered systemically or locally to the cancer.

61. The method of any one of claims 45-60, wherein the cancer vaccine and/or the pharmaceutical composition is administered by intravenous, intratumoral, intramuscular, or subcutaneous administration.

62. The method of any one of claims 45-61, wherein the cancer vaccine and/or the pharmaceutical composition is administered to the subject conjointly with an immunotherapy and/or cancer therapy, optionally wherein the immunotherapy and/or cancer therapy is administered before, after, or concurrently with the cancer vaccine.

63. The method of claim 62, wherein the immunotherapy inhibits an immune checkpoint.

64. The method of claim 63, wherein the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

65. The method of claim 63 or 64, wherein the immune checkpoint is PD1, PD-L1, or CD47.

66. The method of claim 62, wherein the cancer therapy is selected from radiation, a radiosensitizer, and a chemotherapy.

67. The method of any one of claims 45-66, wherein the cancer is a solid or hematological cancer.

68. The method of any one of claims 45-67, wherein the cancer is selected from multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and glioblastoma.

69. The method of any one of claims 45-68, wherein the cancer is selected from oral cancer, pancreatic cancer, lung cancer, colon cancer, melanoma, breast cancer, ovarian cancer, and glioblastoma.

70. The method of any one of claims 45-69, wherein the cancer vaccine and/or the pharmaceutical composition is administered at least twice to the subject, optionally wherein the cancer vaccine and/or the pharmaceutical composition are administered to the subject after at least one month since the first administration.

71. The method of any one of claims 45-70, wherein the subject is a mammal, optionally wherein the mammal is a mouse or human.

72. A method of activating NK cells in a subject in need thereof, comprising administering to the subject
a) the cancer vaccine or the pharmaceutical composition of any one of claims 1-44 and/or
b) NK cells activated by contacting the NK cells with the cancer vaccine or the pharmaceutical composition of any one of claims 1-44.

73. The method of claim 72, wherein the method comprises administering to the subject
a) the cancer vaccine or the pharmaceutical composition of any one of claims 1-44; and
b) NK cells activated by contacting the NK cells with the cancer vaccine or the pharmaceutical composition of any one of claims 1-44.

74. The method of claim 72 or 73, wherein the method is part of an adjuvant therapy.

75. The method of any one of claims 72-74, wherein the subject is afflicted with a cancer.

76. The method of claim 75, wherein the cancer cells in the cancer vaccine are derived from a cancer of the same type as the cancer treated with the cancer vaccine.

77. The method of claim 75, wherein the cancer cells in the cancer vaccine are derived from a cancer of a different type than the cancer treated with the cancer vaccine.

78. The method of any one of claims 72-77, wherein the cancer vaccine and/or the pharmaceutical composition is syngeneic or xenogeneic to the subject.

79. The method of any one of claims 72-78, wherein the cancer vaccine and/or the pharmaceutical composition is autologous, matched allogeneic, mismatched allogeneic, or congenic to the subject.

80. The method of any one of claims 72-79, wherein the NK cell is a primary NK cell.

81. The method of any one of claims 72-80, wherein the cancer vaccine and/or the pharmaceutical composition expands the NK cell, optionally wherein the NK cell expands to at least about 10, 11, 12, 13, 14, 15, 16, 17, 18, 19, 20, 21, 22, 23, 24, 25, or more population doublings within 4 weeks.

82. The method of any one of claims 72-81, wherein the cancer vaccine and/or the pharmaceutical composition enhances NK cell cytotoxicity.

83. The method of any one of claims 72-82, wherein the cancer vaccine and/or the pharmaceutical composition increases or promotes production, secretion, and/or function of at least one cytokine or chemokine produced by the NK cell.

84. The method of claim 83, wherein the at least one cytokine is IFN-γ.

85. The method of any one of claims 72-84, wherein the NK cell expands a CD8+ T cell.

86. The method of any one of claims 72-85, wherein the NK cell preferentially expands the CD8+ T cell relative to a CD4+ T cell.

87. The method of any one of claims 75-86, wherein the cancer vaccine and/or the pharmaceutical composition is administered systemically or locally to the cancer.

88. The method of any one of claims 72-87, wherein the cancer vaccine and/or the pharmaceutical composition is administered by intravenous, intratumoral, intramuscular, or subcutaneous administration.

89. The method of any one of claims 72-88, wherein the cancer vaccine and/or the pharmaceutical composition is administered to the subject conjointly with an immunotherapy and/or cancer therapy, optionally wherein the immunotherapy and/or cancer therapy is administered before, after, or concurrently with the cancer vaccine.

90. The method of claim 89, wherein the immunotherapy inhibits an immune checkpoint.

91. The method of claim 90, wherein the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

92. The method of claim 90 or 91, wherein the immune checkpoint is PD1, PD-L1, or CD47.

93. The method of claim 89, wherein the cancer therapy is selected from radiation, a radiosensitizer, and a chemotherapy.

94. The method of any one of claims 75-93, wherein the cancer is a solid or hematological cancer.

95. The method of any one of claims 75-94, wherein the cancer is selected from multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and glioblastoma.

96. The method of any one of claims 75-95, wherein the cancer is selected from oral cancer, pancreatic cancer, lung cancer, colon cancer, melanoma, breast cancer, ovarian cancer, and glioblastoma.

97. The method of any one of claims 72-96, wherein the cancer vaccine and/or the pharmaceutical composition is administered at least twice to the subject, optionally wherein the cancer vaccine and/or the pharmaceutical composition are administered to the subject after at least one month since the first administration.

98. The method of any one of claims 72-97, wherein the subject is a mammal, optionally wherein the mammal is a mouse or human.

99. A method of activating an NK cell in vitro or ex vivo comprising contacting the NK cell with the cancer vaccine and/or the pharmaceutical composition of any one of claims 1-44.

100. The method of claim 99, wherein the NK cell is a primary NK cell.

101. The method of claim 99 or 100, wherein the cancer vaccine and/or the pharmaceutical composition is syngeneic or xenogeneic to the NK cell.

102. The method of any one of claims 99-101, wherein the cancer vaccine and/or the pharmaceutical composition is autologous, matched allogeneic, mismatched allogeneic, or congenic to the NK cell.

103. The method of any one of claims 99-102, wherein the cancer vaccine and/or the pharmaceutical composition expands the NK cell, optionally wherein the NK cell expands to at least about 10, 11, 12, 13, 14, 15, 16, 17, 18, 19, 20, 21, 22, 23, 24, 25, or more population doublings within 4 weeks.

104. The method of any one of claims 99-103, wherein the cancer vaccine and/or the pharmaceutical composition enhances NK cell cytotoxicity.

105. The method of any one of claims 99-104, wherein the cancer vaccine and/or the pharmaceutical composition increases or promotes production, secretion, and/or function of at least one cytokine or chemokine produced by the NK cell.

106. The method of claim 105, wherein the at least one cytokine is IFN-γ.

107. The method of any one of claims 99-106, wherein the NK cell expands a CD8+ T cell.

108. The method of any one of claims 99-107, wherein the NK cell preferentially expands the CD8+ T cell relative to a CD4+ T cell.

109. The method of any one of claims 99-108, wherein the NK cell is a mammalian cell, optionally wherein the mammalian cell is a mouse cell or a human cell.

110. The NK cells activated by the method of any one of claims 99-109.

111. A pharmaceutical composition comprising the NK cells of claim 110.
